(19) United States
(12) Patent Application Publication
(10) Pub. No.: US 2007/0276025 A1
(43) Pub. Date: Nov. 29, 2007

(54) Indirubin-Type Compounds, Compositions, and Methods for Their Use

(76) Inventors: Laurent Meijer, Roscoff (FR); Paul Greengard, New York, NY (US); Marie Knockaert, Roscoff (FR); Alexios-Leandros Skaltounis, Athens (GR)

Correspondence Address: JONES DAY, 222 EAST 41ST ST, NEW YORK, NY, 10017, US

(21) Appl. No.: 10/577,686

(22) PCT Filed: Oct. 28, 2004
(86) PCT No.: PCT/US04/35931

§ 371 Date: Jun. 28, 2007

Related U.S. Application Data
(60) Provisional application No. 60/515,571, filed on Oct. 28, 2003.

(51) Int. Cl. A61K 31/404; A61P 25/28; A61P 3/10; C07D 403/04; C09B 7/02; C09B 7/04; C12N 9/99

(52) U.S. Cl. 514/414; 435/184; 435/375; 548/457; 548/459

(57) ABSTRACT

Compounds and compositions including 6-bromo-indirubin, 5-amino-indirubin and N-methyl-indirubins and related indirubin derivatives are provided that are useful as selective modulators of glycogen synthase kinase-3, cyclin-dependent protein kinases or aryl hydrocarbon receptors. Methods of inhibiting or modulating cell growth or cell cycling are provided using the compounds of the invention. In other aspects, compounds and methods for the treatment of protozoan-mediated diseases, Alzheimer's disease and diabetes are provided.

This application claims the benefit under 35 U.S.C. § 19(e) of U.S. provisional application No. 60/515,571, filed Oct. 28, 2003.

### 1. FIELD OF THE INVENTION

The field of the invention relates to bis-indole or indirubin-type compounds and compositions useful as selective modulators of protein kinases and aryl hydrocarbon receptors. More specifically, the invention provides 6-substituted indirubins useful as inhibitors of glycogen synthase kinase-3. The invention also provides N-methyl-indirubins useful as selective modulators of the aryl hydrocarbon receptor. In another aspect, the invention provides methods of screening compounds for selective modulatory activity on glycogen synthase kinases, cyclin-dependent protein kinases and/or aryl hydrocarbon receptors. In another aspect, the invention provides methods for the treatment of conditions and disorders associated with cancer, pancreatic cancer, leukemia, chronic myelocytic leukemia, neurodegenerative disorders, Alzheimer's disease, bipolar disorder, infectious diseases such as malaria and other protozoan-derived diseases, and diabetes.

### 2. BACKGROUND

Bis-indole indirubins have been identified as a main active ingredient of a traditional Chinese medicinal recipe, Danggui Longhui Wan, used to treat various diseases including chronic myclocytic leukemia (Tang & Eisenbrand (1992) Chinese drugs of plant origin, chemistry, pharmacology, and use in traditional and modern medicine (Springer-Verlag: Heidelberg); Xiao et al. (2002) Leuk. Lymphoma 43: 1763-1768). Indirubins contribute by their red color to the unique blue-purplish color of natural indigo, which distinguishes it from synthetic indigo. Indirubins can be extracted from various natural sources such as indigo-producing plants (Maugard et al. (2001) Phytochem. 58: 897-904), several species of Gastropod mollusks (Cooksey (2001) Molecules 6: 736-769), urine in healthy and diseased patients (Adachi et al. (2001) J. Biol. Chem. 276: 31475-31478), and various wild-type or recombinant bacteria (Gillam et al. (2000) Biochem. 39: 13817-13824; MacNeil et al. (2001) J. Mol. Microbiol. Biotechnol. 3: 301-308).

Interest in this family of bis-indole compounds increased when indirubin and analogues (collectively referred to as indirubins) were found to inhibit the cell cycle regulating cyclin-dependent kinases (“CDKs”) (Hoessel et al. (1999) Nature Cell Biol. 1: 60-67), and glycogen synthase kinase-3 (“GSK-3”) (Leclerc et al. (2001) J. Biol. Chem. 276: 251-260). There are suggestions that the anti-proliferative effects of indirubins derive from their ability to inhibit CDKs (Marko et al. (2001) Br. J. Cancer 84: 283-289; Damiens et al. (2001) Oncogene 20: 3786-3797). However, in addition to inhibition of CDKs and GSK-3, indirubins have been reported to activate the aryl hydrocarbon receptor (“AhR”), also known as the dioxin receptor (Adachi et al. (2001) J. Biol. Chem. 276: 31475-31478). Thus, the exact mechanism by which indirubins act is not clear. Moreover, abnormal regulation of CDKs, GSK-3 and AhR underlies certain pathologies, as explained below, necessitating the elucidation of potent, selective pharmacological inhibitors.

CDKs (such as CDK1, 2, 4, 6) control progression through the cell division cycle and apoptosis, and appear to be deregulated in many human cancers (Malumbres et al. (2000) Biol. Chem. 381: 827-38; Malumbres & Barbacid (2001) Nature Reviews Cancer 1: 222-231; Ortega et al. (2002) Biochim. Biophys. Acta 1602: 73-87). Other CDKs (CDK5, CDK11) are involved in various functions in the nervous system. CDK5 is abnormally regulated in Alzheimer's Disease (“AD”) and other neurodegenerative disorders (Dhavan & Tsai (2001) Nature Rev. Mol. Cell Biol. 2: 749-759; Maccioni et al. (2001) Eur. J. Biochem. 268: 1518-1527; Smith & Tsai (2002) Trends Cell Biol. 12: 28-36).

In mammals different genes encode two closely related glycogen synthase kinase-3 (GSK-3), GSK-3α and GSK-3β. GSK-3 functions in Wnt signal transducing pathways, insulin action, apoptosis or programmed cell death, tumorigenesis, and circadian rhyttum, and misregulation of GSK-3 activity is implicated in various human diseases (diabetes, AD, cancers) (Cohen & Frame (2001) Nature Rev. Mol. Cell Biol. 2: 769-776; Grimes & Jope (2001) Progress Neurobiol. 65: 391-426; Eldar-Finkelman (2002) Trends Mol. Medic. 8: 126-132; Kaytor & Orr (2002) Curr. Opinion Neurobiol. 12: 275-278; Nikoulina et al. (2002) Diabetes 51: 2190-2198; De Strooper & Woodgett (2003) Nature 423: 392-393). Growing evidence supports the view that GSK-313 activation and nuclear translocation are a prerequisite for neuronal apoptosis (Ding & Dale (2002) Trends Biochem. Sci. 27: 327-329; Li et al. (2002) Bipolar Disord. 4: 137-144; Bijur & Jope (2001) J. Biol. Chem. 276: 37436-37442). Cell culture experiments have shown that GSK-3 inactivation prevents apoptosis induced by various agents. For example, overexpression of FRAT1, a negative regulator of GSK-3, confers resistance of PC12 cells to apoptosis (Crowder & Freeman (2000) J. Biol. Chem. 275: 34266-34271; Culbert et al. (2001) FEBS Lett. 507: 288-294), pharmacological inhibitors of GSK-3 prevent apoptosis in PC12 cells, human SH-SY5Y neuroblastoma cells, and a Huntington disease cell model (Bhat et al. (2000) Proc. Natl. Acad. Sci. U.S.A. 97: 11074-11079; Cross et al. (2001) J. Neurochem. 77: 94-102; Bijur et al. (2000) J. Biol. Chem. 275: 7583-7590; Song et al. (2002) J. Biol. Chem. 277: 44701-44708; Carmichael et al. (2002) J. Biol. Chem. 277: 33791-33798), activation of Wnt signaling (and therefore GSK-3 inhibition) prevents c-Myc induced apoptosis in Rat-1 cells (You et al. (2002) J. Cell Biol. 157: 429-440), expression of a kinase-deficient GSK-3β reduces cell death induced by Akt inhibition (Crowder & Freeman (2000) J. Biol. Chem. 275: 34266-34271), and FGF (which activates Akt and thus inhibits GSK-3) reduces cell death induced by glutamate (Hashimoto et al. (2002) J. Biol. Chem. 277: 32985-32991) while overexpression of GSK-3β increases cell death. In addition to these in vitro data, two animal models reinforce the link between GSK-3 activation and apoptosis: (a) conditional transgenic mice overexpressing GSK-3 in brain during adulthood show signs of neuronal stress and apoptosis (Lucas et al. (2001) EMBO J. 20: 27-39), (b) expression of shaggy, the Drosophila homologue of GSK-3β, enhances the neurodegeneration induced by expression of human Tau in flies, while expression of a loss-of-function mutant of shaggy prevents this neurodegeneration (Jackson et al. (2002) Neuron 34: 509-519).

Only a limited number of pharmacological inhibitors of GSK-3 are available (Martinez et al. (2002) Medic. Res. Rev. 22: 373-384), lithium being the most frequently used (Davies et al. (2000) Biochem. J. 351: 95-105), despite its effects being in the 10-20 mM range and its demonstrated effect on inositol phosphatases (Patel et al. (2002) Mol. Biol. 315: 677-685).

CDK5 and GSK-3 are two main kinases involved in the abnormal hyper-phosphorylation of the microtubule-binding protein tau, one of the diagnostic features of AD. Recently, it was demonstrated that transgenic mice co-expressing both mutant human (P301L) tau and p25, the CDK5 activator, show an accumulation of aggregated, hyperphosphorylated tau, associated with GSK-3, and increased neurofibrillary tangles (Noble et al. (2003) Neuron 38: 555-565). Studies also established that GSK-3 is involved in the production of amyloid-β peptides, another event thought to be directly involved in AD's development (De Strooper & Woodgett (2003) Nature 423: 392-393; Phiel et al. (2003) Nature 423: 435-439). These observations constitute a strong encouragement for the search for GSK-3/CDK5 inhibitors as potential pharmacological agents to treat AD.

AhR is a member of the bBLH/PAS family of transcriptional regulators that mediates the effects of many xenobiotics such as 2,3,7,8-tetrachlorodibenzo-p-dioxin (“TCDD”) and indole-containing compounds (Hankinson (1995) Annu. Rev. Pharmacol. Toxicol. 35: 307-340; Denison & Nagy (2003) Annu. Rev. Pharmacol. Toxicol. 43: 309-334). Deregulation of AhR leads to the appearance of stomach tumors (Andersson et al. (2002) Proc. Natl. Acad Sci. USA 99: 9990-9995). On the other hand, some AhR agonists have been reported to cause growth inhibition of various tumor cell lines indicating that AhR agonists could be evaluated for their use in anti-tumor therapy (Bradshaw et al. (2002) Curr. Pharmac. Design 8: 2475-2490; Koliopanos et al. (2002) Oncogene 21: 6059-6070 Safe & McDougal (2002) Int. J. Oncol. 20: 1123-1128). The binding of AhR to a ligand leads to its translocation from the cytoplasm to the nucleus, followed by complex formation with the aryl hydrocarbon receptor nuclear translocator (ARNT). This complex then binds to xenobiotic-responsive element (XRE) and stimulates the transcription of a wide variety of genes, including cytochrome P450 Cyp1A1, p27^{kip1}, myristoyltransferase, among others (Rowlands & Gustafsson (1997) Crit. Rev. Toxicol. 27: 109-134; Kolluri et al. (1999) Genes & Dev. 13: 1742-1753; Kolluri et al. (2001) Cancer Res. 61: 8534-8539; Santini et al. (2001) J. Pharmacol. Exp. Ther. 299: 718-728; Denison et al. (1998) in “Xenobiotics, receptors and Gene Expression” (Denison & Helferich, eds., Taylor and Francis, Philadelphia) pp. 3-33). Several arguments support a link between AhR activation and cell cycle control (Ge & Elferink (1998) J. Biol. Chem. 273: 22708-22713; Elferink et al. (2001) Mol. Pharmacol. 59: 664-673). The discovery of the interaction of indirubin with AhR opened the possibility that indirubins prevent cell proliferation via an action through AhR (Adachi et al. (2001) J. Biol. Chem. 276: 31475-31478).

Ligand binding has been mapped to the second PAS domain of the AhR protein (Fukunaga et al. (1995) J. Biol. Chem., 270: 29270-29278). A model for recognition of TCDD by AhR, based on the crystal structure of the conserved PAS domain of the heme-binding domain of the bacterial O₂ sensing FixL protein, has been proposed (Procopio et al. (2002) Eur. J. Biochem. 269: 13-18). Another 3D model of AhR has been published recently (Jacobs et al. (2003) J. Steroid Biochem. Mol. Biol. 84: 117-132). According to these models AhR should be able to accommodate the flat hydrophobic indirubins in a way similar to TCDD.

Selective modulation of CDKs, GSK-3 or AhR would be beneficial for the targeted pharmacological manipulation of one pathway without unintended consequences associated with modulation of a second pathway. For example, given the diverse effects of AhR signaling versus GSK-3 signaling there is a need for indirubin-type compound that act selectively upon AhR and not protein kinases, and vice versa. Screening methods for rapid identification of modulatory abilities-across a series of targets, e.g., CKss, GSK-3 and ArH, are therefore also desirable.

Citation or identification of any reference in Section 2, or in any other section of this application, shall not be considered an admission that such reference is available as prior art to the present invention.

### 3. SUMMARY OF THE INVENTION

The present invention provides compounds useful for the selective modulation of CDK, GSK-3 and/or ArH. In part, the present invention is based upon the surprising discovery that indirubins with a halogen attached to the number 6 carbon, or C6, of indirubin (see, e.g., FIG. 1) inhibit GSK-3 with IC₅₀ values that are well below the IC₅₀ values that same halogen-bearing indirubins inhibit CDKs, such as CDK5. The differences in IC₅₀ values ranging from a 10-fold difference to over a 1000-fold difference as observed, for example, in Table 5.

In one aspect, the present invention is an isolated compound selected from the group consisting of 6-bromoindirubin (5a), 6,6′-dibromoindirubin (12b), 6-bromoindirubin-3′-oxime (“BIO”) (7a), 6,6′-dibromoindirubin-3′-oxime (13b), 6-bromoindirubin-3′-methoxime (9a), 6-bromo-5-methylindirubin (5) and 6-bromoindirubin-3′-acetoxime (8a), and pharmaceutically acceptable salts thereof, useful for the selective inhibition of GSK-3 as opposed to modulation of CDK activities.

In another aspect, the present invention is an isolated compound selected from the group consisting of 6-bromoindirubin (5a), 6,6′-dibromoindirubin (12b), 6-bromoindirubin-3′-oxime (“BIO”) (7a), 6,6′-dibromoindirubin-3′-oxime (13b), 6-bromoindirubin-3′-methoxime (9a), and 6-bromoindirubin-3′-acetoxime (8a), and pharmaceutically acceptable salts thereof, useful for inhibiting GSK-3 with an IC₅₀ value of less than 5 μM, less than 0.5 μM, or of less than 0.1 μM.

In certain embodiments, the compound is 6-bromoindirubin (5a), 6,6′-dibromoindirubin (12b), 6-bromoindirubin-3′-oxime (“BIO”) (7a), 6,6′-dibromoindirubin-3′-oxime (13b), 6-bromoindirubin-3′-methoxime (9a), 6-bromo-5-methylindirubin (5), 6-bromo-5-aminoindirubin (27), 6-bromo-5-amino-3′-oxime-indirubin (28), 6-bromoindirubin-3′-acetoxime (8a), 5-amino-indirubin (23), 5-amino-3′-oximeindirubin (24) or pharmaceutically acceptable salts thereof.

In another aspect, the present invention is an isolated compound consisting of indirubin substituted with a halogen at C6 of the indirubin, or indirubin-3′-oxime substituted with a halogen at C6 of the indirubin, or pharmaceutically acceptable salts thereof, useful for inhibiting GSK-3 with an IC₅₀ value of less than 0.1 μM.

In part, the present invention is based upon the surprising discovery that indirubins with an oxime group attached to C3′ of indirubin and indirubin-type compounds improves inhibitory potency for inhibition of protein kinases. In one aspect, the present invention is an indirubin with a 3′-oxime, or pharmaceutically acceptable salts thereof useful for inhibiting protein kinases with an IC₅₀ value of less than 0.1 μM.

In another aspect, the present invention is an isolated compound useful for the selective activation of ArH wherein the compound is selected from the group of compounds consisting of 1-methylindirubin (12d), 1-methylindirubin-3′-oxime (“MeIO”) (13d), 1-methyl-6-bromoindirubin (12c), and 1-methyl-6-bromoindirubin-3′-oxime (“MeBIO”) (13c), or pharmaceutically acceptable salts thereof. By selective activation of ArH, it is meant that a given compound activates ArH with an EC₅₀ in submicromolar concentrations, i.e., less than 1 μM, wherein the same compound inhibits GSK-3 or CDK5 activities with an IC₅₀ greater than 10 μM.

### 4. BRIEF DESCRIPTION OF THE FIGURES

FIG. 1 illustrates the chemical structure of indirubin (5h) ( FIG. 1A) and exemplary substituted indirubin compounds (FIG. 1B).

FIGS. 2A and B illustrate Scheme 1 and Scheme 2 for the preparation of exemplary compounds.

FIGS. 3A and B illustrates synthetic schemes for exemplary 5′-amino-indirubin compounds.

FIG. 4 depicts indirubin immobilized on Affigel through a linker (FIG. 4A); indirubin beads (FIG. 4B) and control ethanolamine beads were exposed to a porcine brain lysate in the presence (+) or absence (−) of 20 μM BIO, and following stringent washing, the bound proteins were analyzed by SDS-PAGE followed by silver staining (FIG. 4C) or Western blotting with anti-GSK-3 antibody (FIG. 4D) thereby demonstrating that BIO is a selective GSK-3 inhibitor in vitro.

FIG. 5 depicts the structure of GSK-3β-BIO co-crystal: FIG. 5A shows ribbon diagram showing the overall fold of the GSK-3β-BIO complex in which the BIO molecule is shown as a stick model with carbon atoms colored light-gray, oxygen atoms red, nitrogen atoms blue and bromine in green and the position of the N-terminus is indicated; FIG. 5B shows a 2Fₒ-F_{c} omit density map of the BIO contoured at 2σ; a LIGPLOT (Wallace et al. (1995) Protein Eng. 8: 127-134) interaction diagram for BIO with hydrogen bonds shown as dotted lines and Van der Waals contacts shown as ‘hairy’ semi-circles is shown in FIG. 5C; and a superposition of CDK2/cyclinA-I5S with GSK-3β-BIO with CDK complex in green, GSK-3 complex in gray is presented in FIG. 5D (FIGS. 5A, 5B and 5D were created with PyMol (DeLano, W. L. (2002) The PyMOL Molecular Graphics System (2002). DeLano Scientific, San Carlos, Calif., USA.).

FIG. 6 depicts the structure of CDK5/p25-IO co-crystal: FIG. 6A shows a ribbon diagram showing the overall fold of the CDK5/p25-IO complex where the IO molecule is shown in a ball-and-stick representation with carbon atoms colored light-gray, oxygen atoms red and nitrogen atoms blue and the position of the N- and C-termini are indicated (created with program RIBBONS (Carson, M. (1991). Ribbons 2.0. J. Appl. Crystallography 24, 958-961)); FIG. 6B shows a 2Fₒ-F_{c} omit electron density map for IO contoured at one time the root mean square deviation (1 σ) of the map; FIG. 6C presents a schematic diagram of the interaction of IO with CDK5/p25 where hydrogen bonds are shown as dotted lines and Van der Waals contacts are shown as ‘hairy’ semi-circles (created with program LIGPLOT ); and FIG. 6D shows a superposition of CDK5/p25-IO and CDK2/cyclinA-I5S obtained considering the kinase residues adjacent to the inhibitors. IO is colored as in panel A, while atoms in CDK2/cyclinA-I5S are colored in green.

FIG. 7 illustrates that BIO is a selective GSK-3 inhibitor in cell cultures: A. BIO inhibits the phosphorylation of β-catenin on GSK-3 specific sites. Cos-1 cells were untreated (Mock) or exposed to 5 μM IO , BIO, MeBIO, or 20 mM LiCl for 24 hrs. Proteins were then separated by SDS-PAGE followed by Western blotting with antibodies directed (top to bottom) against β-catenin, dephospho-β-catenin, GSK-3 and actin (loading control). A non-specific band detected with the dephospho-β-catenin was used as an additional loading control. B. Independence from AhR. Cell lines deficient either for AhR or ARNT were exposed to 10 μM BIO, 50 μM MeBIO for 24 hrs. Western blots were then made with antibodies directed against β-catenin and actin (loading control). C. Indirubins inhibit the tyrosine phosphorylation of GSK-3. SH-SYSY cells were untreated (Mock) or exposed to 1 μM IO, MeIO, BIO and MeBIO for 12 hr. Proteins were then separated by SDS-PAGE followed by Western blotting with antibodies directed (top to bottom) against total GSK-3β, phospho Tyr276 (GSK-3α) /Tyr216 (GSK-3β), dephospho-β-catenin and total β-catenin.

FIG. 8 illustrates that BIO activates the maternal Wnt signaling pathway in Xenopus laevis embryos. A. Diagram of the Wnt pathway in Xenopus. The site of action and mechanism for Wnt pathway activation are indicated. Green arrows indicate positive effects, and red capped lines indicate negative ones. B. Untreated embryo, tadpole stage. C-G. Activation of the maternal Wnt pathway. Embryos were treated with the specified reagents before stage 8, and were allowed to develop to tadpole stage. C. Embryos treated with 50 μM of the inactive MeBIO are unchanged. D. Embryos treated with 0.3 M LiCl are anteriorized. BEG. Dose-dependent effect of BIO. The intensity of the anteriorized phenotype increases with BIO concentration (50 μM, 15 μM, and 5 μM respectively). H. BIO activates ectopically the dorsal genes siamois and chordin, and epistasis analysis of their induction is consistent with in vivo inhibition of GSK-3. RT-PCR of animal cap explants for the direct Wnt target gene siamois and the siamois target chordin, with odc as loading control. Li⁺ (0.3 M) and BIO (50 μM) induce both siamois and chordin (lanes 3, 4), which are absent in explants from untreated embryos (lane 2). The GSK-3-independent inhibitor of the Wnt pathway DN-Xtcf-3 blocks completely the effect of Li⁺ (lane 6), and partially the effect of BIO (lane 7). The GSK-3-dependent inhibitor axin fails to block either Li⁺ (lane 9, compare to 3), or BIO (lane 10, compare to 4). I. BIO is a potent anterior and neural tissue inducer in animal cap explants. RT-PCR of animal cap explants for general neural (nrp1), anterior neural (otx2), anterior tissue (cement gland marker xag1), midbrain (en2), and posterior neural (xhoxb9) markers. Lane 1 is a control embryo, stage 16. Cap explants from untreated embryos do not express anterior or neural markers (lane2). Animal cap explants from embryos treated with BIO (lanes 6, 7) express anterior neural markers more effectively than explants from embryos treated with LiCl (lanes 4, 5), or injected with RNA for the neural inducer noggin (lane 3). odc is a loading control marker.

FIG. 9. Crystal structure of indirubin-3′-oxime in complex with CDK5/p25 (a, c) and of 6-bromoindirubin-3′-oxime with GSK-3β (b, d). Inhibitor bind in the ATP-binding pocket of the catalytic site, mainly through hydrophobic interaction and three hydrogen bonds with Cys 83 and Cys81 (CDK5) or Val 135 and Asp133 (GSK-3 β). The CDK5 co-factor, p25, is the upper right lobe (shown in dark gray) of the structure shown in FIG. 9A. Indirubins are shown as ball-and-stick models. The Figures were created with the molecular viewer of AutoDockTools and Macromodel. 100291 FIG. 10. Correlation between experimental and predicted GSK-3β ΔG values of the indirubins. ◯, training set; ●, test set.

FIG. 11. FIG. 11A: A plot of the values of the three individual energy terms (Van der Waals, electrostatic, hydrogen bond) versus the total interaction energy as calculated by PrGen. FIG. 11B. Comparison of the contribution of the electrostatic and hydrogen bond term expressed by the ratio (elect.+HB)/Eₜₒₜₐₗ among pairs of oxime and corresponding non-oxime substituted indirubins.

FIG. 12. Superimposition of CDK2-I5S and CDK2-ATP. In the co-crystal structure of CDK2-ATP the two hydroxyl groups of the ribose moiety of ATP form hydrogen bonds with the side chain of Asp86. In the structure of CDK2-indirubin 5-sulphonate a water molecule is located approximately at the same position of those hydroxyl groups. This water molecule could bridge the C3′ oxygen with the side chain of Asp86 through the formation of hydrogen bonds, mimicking the interaction formed between the natural substrate and the receptor.

FIG. 13. Superimposition of CDK5-IO and GSK-3β-BIO. An indirect interaction of the oxime with the side chain of Asp86 in the CDK5 structure requires one bridging water molecule (water 79). In the GSK-3β structure, Asp86 is replaced by a threonine (Thr138). In this case two bridging water molecules (water 49 and 79) are required to preserve an interaction between the oxime of BIO and the hydroxyl of Thr138.

FIG. 14. Indirubins are potent AhR agonists. Increasing concentrations of indirubins and TCDD were tested in a hepatoma cell line reporter system, expressing EGFP under the control of a dioxin-responsive element which binds to ligand- AhR-ARNT complex. EGFP activity is reported as a percentage of maximal activity obtained with 1 nM TCDD.

FIG. 15A-L. Indirubins induce nuclear translocation of AhR. Upper panel: Wild-type Hepa-1 cells were treated for 90 min. with either DMSO as a control (A, B, C) or 25 μM IO (D, E, F). The cells were fixed and AhR distribution was detected by indirect immunofluorescence microscopy using anti-AhR antibody (A, C and D, F). Total DNA was stained with DAPI (B, C and E, F). Lower panel: Wild-type Hepa-1 (G, H, I) and ARNT mutant (J, K, L) cells were treated for 90 min. with either DMSO as a control (G, J) or 10 nM TCDD (H. K) or 25 μM MeIO (I, L). Intracellular distribution of AhR was analyzed as described above. Scale bar, 10 μm.

FIG. 16. Characterization of AhR −/− (BP8) and AhR +/+ (5L) cell lines. The presence and absence of AhR expression in 5L and BP8 cells was confirmed by Western blotting using anti-AhR antibody.

FIG. 17. Effects of indirubins on the proliferation of AhR −/− (BP8) and AhR +/+ (5L) cells. 5L (A) and BP8 (B) cells were treated with 20 μM IO, 20 μM MeIO or a corresponding amount of the carrier DMSO (control) for indicated times. Cell proliferation was estimated by direct counting and the graph shows a representative of three independent experiments with each data point done in triplicates (average and S.E.).

FIG. 18. Effects of indirubins on the survival of AhR −/− (BP8) and AhR +/+ (5L) cells. 5L and BP8 cells were maintained in the presence of increasing concentrations of IO, BIO, MeIO and MeBIO for 48 hrs. The cell survival was determined by the MT assay as described in the Examples and is presented as a percent of control, non-treated cells. The graphs show a representative of three independent experiments with each data point done in triplicates (average and S.E.).

FIG. 19. AhR-active, but kinase-inactive indirubins induce an AhR-dependent arrest in G1 phase. A. 5L and BP8 cells were cultured in the absence (control) or presence of 10 μM MeIO for 24 hr, and the G1, S and G2/M cycle phase distribution was determined by FACS analysis. B. The cell cycle phase distribution of 5L and BP8 cells was quantified following exposure to 0.1 μM TCDD or 10 μM IO, BIO, MeIO or MeBIO for 24 hrs.

FIG. 20. AhR-active, but kinase-inactive indirubins induce an AhR-dependent up-regulation of p27^{KIP1}. A. 5L and BP8 cells were treated with increasing concentrations of MeIO or 0.1 μM TCDD for 24 hr. The expression level of p27^{KIP1} was determined by Western blotting using a specific antibody. B. Determination of p27^{KIP1} levels in 5L and BP8 cells by Western blotting following exposure to 0.1 μM TCDD or 10 μM IO, BIO, MeIO or MeBIO for 24 hrs.

FIG. 21. Model for the dual mechanism of action of indirubins in dividing cells. Indirubins with a high affinity for AhR and low affinity for kinases bind to and activate AhR more readily. The AhR/indirubin complex translocates to the nucleus and forms a dimer with ARNT. This complex then binds to xenobiotic-responsive elements (XRE) present in the regulatory domains of numerous target genes and directly or indirectly stimulates the expression of target genes, e.g. p27^{KIP1}. p27^{KIP1} is a potent inhibitor of CDK2, leading to a marked arrest in Gi and a subsequent cytostatic effect. Indirubins with a higher affinity for protein kinases inactivate CDKs, GSK-3, and probably other kinases. This induces cell cycle arrest at multiple stages, ultimately leading to reduced cell survival and thus a anti-proliferative effect.

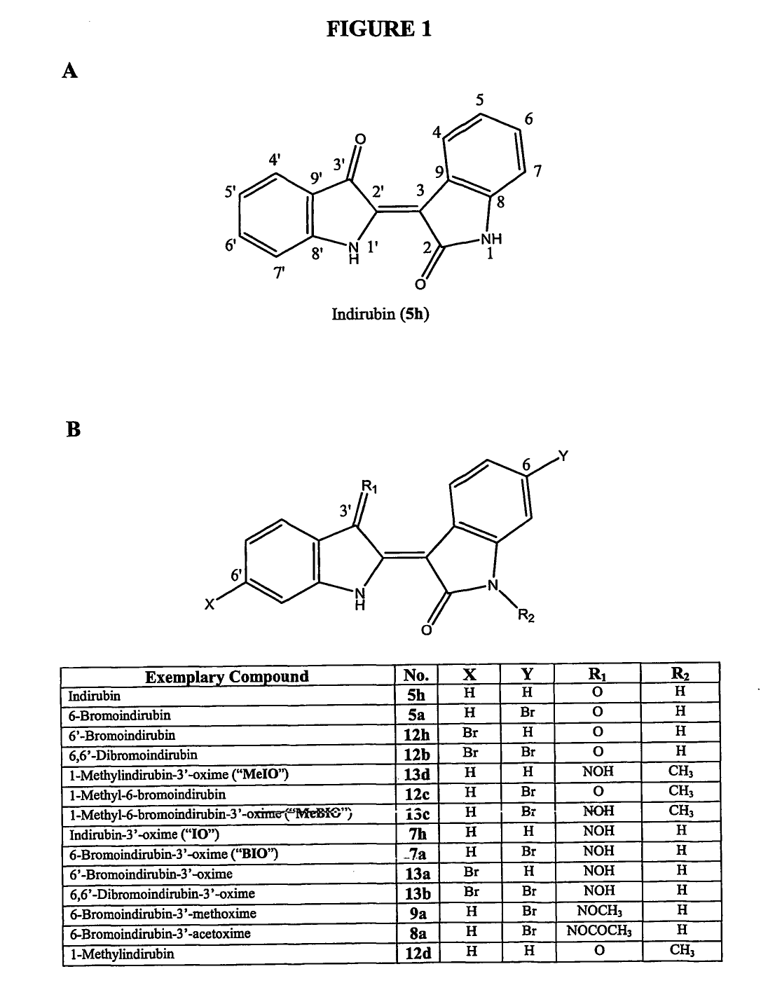

### 5. DETAILED DESCRIPTION OF EMBODIEMTNS OF THE INVENTION

### 5.1. Embodiments of the Invention

Compounds, compositions, and methods are described according to the various embodiments of the invention presented in detail below.

Without intending to bound by any particular theory, the compounds, compositions, and methods are based upon a series of experiments initiated from a study of the natural indirubins isolated from Mediterranean mollusk Hexaplex trunculus resulting in the identification of new CDK, GSK-3 and ArH modulators with increased potency and selectivity. Among the various bromo-substituted indirubins identified, 6-bromoindirubin (5a), isolated for the first time from a natural source, turned out to be a potent GSK-3 inhibitor. Using the co-crystal structures of various indirubins with GSK-3β, CDK2 and CDK5/p25, the binding of indirubins within the ATP-binding pocket of these kinases was modeled to pinpoint the specific interactions that contribute both to inhibitory efficacy and to kinase selectivity. Predicted molecules, including 6-substituted and 5,6-disubstituted indirubins, as well as cell-permeable derivatives, such 6-bromoindirubin-3′-oxime were synthesized and evaluated as CDK, GSK-3 and AhR modulators. As determined by performing cellular-based models of CDK, GSK-3 and AhR signaling, distinct indirubin subclasses are described herein that have growth inhibitory effects that are AhR-independent and protein kinase-dependent, whereas other indirubin subclasses, e.g., 1-methyl-indirubins, are AhR-active in concentrations of 1 μM or lower and induce a cytostatic arrest (G1 phase arrest), but are kinase-inactive at similar concentrations.

### 5.1.1. Compounds

The present invention provides isolated compounds useful as a selective GSK-3 inhibitor. In one embodiment, the isolated compound is 6-bromoindirubin (Sa) or a 6-bromoindirubin derivative or analogue. A preferred embodiment is a 3′-oxime derivative, for instance 6-bromoindirubin-3′-oxime (“BIO”) (7a).

In one aspect, the present invention provides BIO and other related 6-bromoindrubins, 6-chloroindirubins, 6-flouroindirubins, and 6-iodoindirubins as potent inhibitors of GSK-3 useful for the treatment of diabetes, neurodegenerative conditions including Alzheimer's disease (“AD”), Huntington disease, bipolar disorder, infectious diseases such as malaria and other protozoan-derived diseases, apoptosis and cancer.

In another aspect, the present invention provides BIO, MeBIO, and other related 6-bromoindirubins as potent, micromolar pharmacological tools and commericial products, as well as relevant inactive controls (e.g., MeBIO), to investigate the cellular functions of GSK-3.

Useful compounds of the invention have selective GSK-3 inhibitory activity and thus are useful to counteract the pathological effects of GSK-3 overactivity, e.g., tau hyperphosphorylation as in AD. Thus, in one embodiment, the isolated compound is selected from group of compounds consisting of 6-bromoindirubin (5a), 6,6′-dibromoindirubin (12b), 6-bromoindirubin-3′-oxime (7a), 6,6′dibromoindirubin-3′-oxime (13b), 6-bromoindirubin-3′-methoxime (9a), and 6-bromoindirubin-3′-acetoxime (8a). As demonstrated herein, the modification by substitution of bromine on position 6 of indirubin leads to enhanced selectivity for GSK-3 over CDKs. As demonstrated in the Examples, 6-bromo-indirubins have clear anti-proliferative effects on cells.

In another embodiment, the selective GSK-3 inhibitor compound is 6-iodoindirubin (5c) or a 6-iodo-indirubin derivative or analogue, for example, 6-iodoindirubin-3′-oxime (7c) or 6-iodoindirubin-3′-acetoxime (8c).

In another embodiment, the selective GSK-3 inhibitor compound is a 5,6-dihalogen-indirubin, wherein each of the halogens are independently selected from the group consisting of Br, Cl, F, and I.

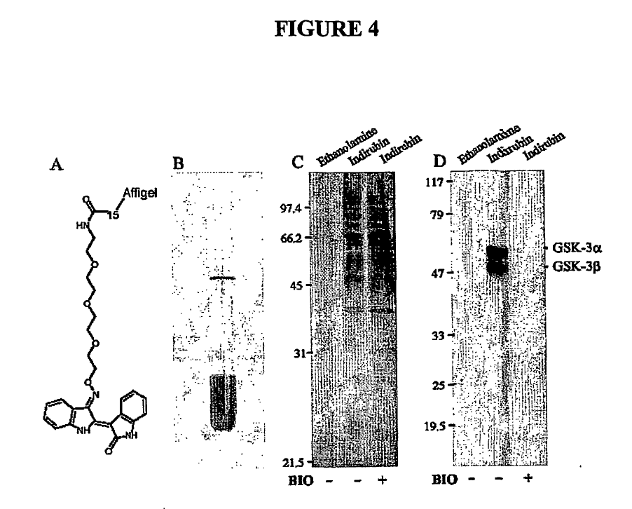

Compared to the non-substituted indirubins, the bromine substitution appears to impart increased global selectivity as seen from the lack of effects of 6-bromo-substituted indirubins on a large panel of kinases (Tables 1 and 5) and from the affinity chromatography experiment (FIG. 4). To illustrate, indirubin (5h) inhibits GSK-3 with an IC₅₀ of 1.0 μM and inhibits CDK5 with an IC₅₀ of 10.0 μM, a 10-fold difference in IC₅₀ values (Table 5). In contrast, for indirubins substituted with bromine, the differences in IC₅₀ concentrations for modulating GSK-3 versus CDK5 are surprisingly greater than the 10-fold difference in IC₅₀ values observed with indirubin (5 h). For example, compound 12b exhibits an IC₅₀ of 4.5 μM with GSK-3 and an IC₅₀ of >100 μM with CKD5, and compound 7a exhibits an IC₅₀ of 0.005 μM with GSK-3 and an IC₅₀ of 0.083 M with CDK5. Thus, in one aspect, selectivity for GSK-3 is shown by a compound of the invention demonstrating greater than a 10-fold difference in an IC₅₀ values in a GSK-3 activity assay versus a CDK5 activity assay, as assessed using the protein kinase assays described herein. In another aspect, a compound of the invention has an IC₅₀ value on GSK-3 of 0.1 μM or less, 0.05 μM or less, or 0.01 μM or less.

In another aspect, the present invention provides any indirubin-type compound that is substituted on C6 with a halogen or vinyl (—CH═CH₂) moiety useful as an inhibitor of GSK-3 with an IC₅₀ of 0.100 μM or less against GSK-3 wherein the indirubin-type compound has an IC₅₀ of 10.0 μM or greater in an CDK5 activity assay. Those of skill in the art recognize standard assays for determination of compounds' IC₅o values on GSK-3 and CDK5 activity assays, for example, the activity assays as described herein in the Examples section. In another aspect, the present invention provides any indirubin-type compound that is substituted on C6 with a halogen or vinyl (—CH═CH₂) moiety useful as an inhibitor of GSK-3, wherein the indirubin-type compound inhibits CDK5 with an IC₅₀ value that is greater than 10-fold than its IC₅₀ value for inhibiting GSK-3. In one embodiment, the indirubin-type compound that is substituted on C6 with a halogen or vinyl moiety is selected from group consisting of compounds 5a, 5c, 5d, 5e, 5f, 5g, 7a, 7c, 7e, 7f, 7g, 8a, 8c, 8d, 8e, 8f, 8g, 9a, 12b, and 13b.

Without intending to bound to any particular theory, the reasons behind this unexpected specificity for GSK-3 are clear in view of the GSK-3β/BIO and CDK5/IO or CDK2/IO co-crystal structures. Briefly, the bromine of BIO establishes a van der Waals contact with the Leu132 residue of GSK-3. The corresponding amino acid in CDK2 and CDK5 is a phenylalanine, i.e., a bulkier residue, which would prohibit the binding of the bromine. In addition, the position 5 of indirubin could accommodate another small substitution compatible with binding to GSK-3, but not to CDK5 or CDK2.

In some embodiments, compounds provided can be a 5-amino-indirubins. In certain embodiments, the compound is selected from the group consisting of 6-bromo-5-aminoindirubin (27), 6-bromo-5-amino-3′-oxime-indirubin (28), 5-amino-indirubin (23), 5-amino-3′-oxime-indirubin (24) and pharmaceutically acceptable salts thereof.

Additional work with molecular modeling, as described in the Examples, was instrumental in determining a class of compounds useful for their ability to activate AhR without significant protein kinase inhibition.

Accordingly, the present invention provides compounds useful for their abilities to effect G1 phase arrest while not inhibiting protein kinases. Specifically, a compound is provided that selectively modulates AhR activity while at the same concentration not affecting GSK-3 or CDK activities. By selective activation of ArH, it is meant that a given compound activates ArH with an EC₅₀ in submicromolar concentrations, i.e., less than 1 μM, using either of two AhR assays as described herein, wherein the same compound inhibits GSK-3 or CDK5 activities with an IC₅₀ values greater than 10 μM, using protein kinase activities as described herein. As described in the Examples, such a compound has clear cytostatic effects which is useful for pharmacological intervention for the treatment of tumors, and other useful purposes without limitation as described herein. In one aspect, the present invention is an isolated compound useful for the selective activation of ArH wherein the compound is selected from the group of compounds consisting of 1-methyl-indirubin (12d), 1-methyl-indirubin-3′-oxime (“MeIO”) (13d), 1-methyl-6-bromo-indirubin (12c), and 1-methyl-6-bromo-indirubin-3′-oxime (“MeBIO”) (13c).

The present invention provides isolated compounds useful as AhR activators that are, in effect, inactive against protein kinases. For example, a given AhR activator of the invention will activate AhR in concentrations ranging from EC₅₀ values of 1 μM to 0.000001 μM or lower, yet the AhR activator's IC₅₀ values for inhibition GSK-3 and/or CDK5 will be 10 μM to 100 μM or greater.

In one embodiment, the isolated compounds consist of 1-methylindirubin (12d) or a 1-methylindirubin derivative or analogue. In a preferred embodiment the isolated compounds useful as AhR activators, while being inactive against or only weakly inhibiting protein kinases, are selected from the group of compounds consisting of 1-methyl-indirubin (12d), 1-methyl-indirubin-3′-oxime (“MeIO”) (13d), 1-methyl-6-bromo-indirubin (12c), and 1-methyl-6-bromo-indirubin-3′-oxime (“MeBIO”) (13c).

Without intending to be bound by any particular theory, among the essential indirubin/kinase bonds, the lactam amide nitrogen of indirubins (N1) donates a hydrogen bond to the backbone oxygen of Glu81 (CDK2), Glu81 (CDK5) or Asp 133 (GSK-3), three amino acids which occupy a homologous position in these kinases. Methylation of indirubins on N1 prohibits this interaction and inactivates the inhibitory properties of indirubins towards these and probably other kinases.

Structure/activity relationship analysis carried out with a large number of synthetic ligands suggests that the AhR ligand-binding pocket can accommodate planar ligands with maximal dimensions of 14 Å×12 Å×5 Å (Denison & Nagy (2003). Annu. Rev. Pharmacol. Toxicol. 43: 309-334). AhR-interacting indirubins, such as MeBIO (12.6 Å×7.9 Å×1.8 Å), clearly meet these requirements.

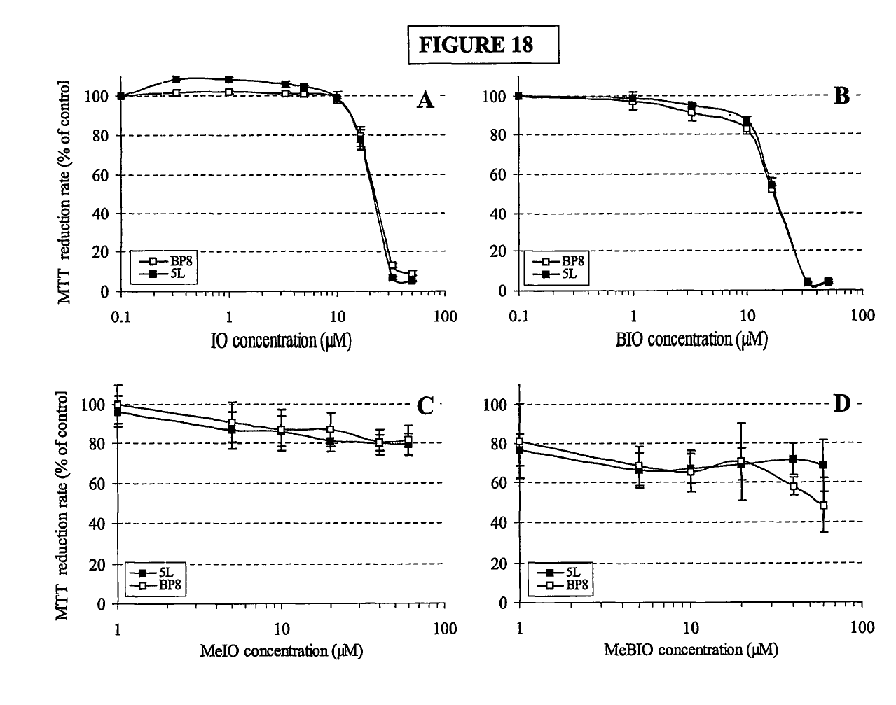

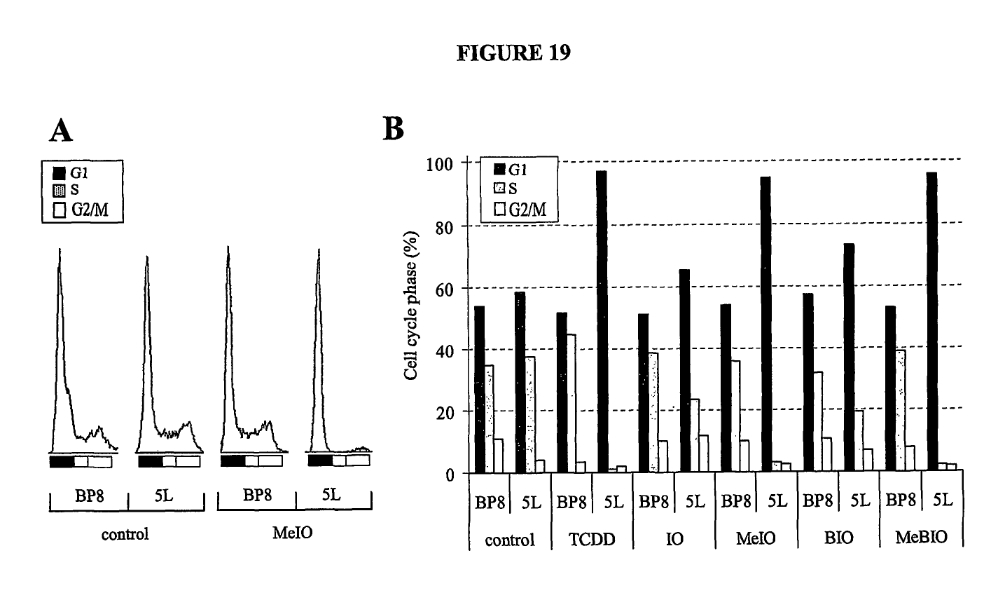

Compared to the non-methylated indirubins, N1-methylated indirubins have -little effect on cell survival as measured by the MTT assay (see Example 6.2.5, below), whether cells express AhR (e.g., 5L cells) or not (e.g., BP8 cells) (FIG. 18). Importantly these N1-methylated indirubins are potent AhR agonists. As such they effect cell proliferation by arresting cells in G1 in a AhR-dependent manner (FIG. 19). Accordingly, indirubins such as IO and BIO reduce cell survival by a mechanism essentially independent from AhR (since similar rates of survival were observed in both 5L and BP8 cells), a mechanism likely to be a direct or indirect consequence of linase inhibition. However both IO and BIO have a modest, but not insignificant effect on AhR as evidenced by the nuclear translocation of AhR they trigger and the limited (but clearly AhR-dependent) p27^{KIP1} induction and G1 arrest they induce. Nevertheless, this limited AhR agonist effect does not contribute to the anti-proliferative effects of these indirubins.

N1-methylated indirubins provide very useful tools to challenge the functions of AhR in cell cycle regulation. These compounds are potent inducers of p27^{KIP1}, and this provides an explanation for the observed G1 arrest. Without intending to be limited by any particular theory, differences in the mechanism of action of methylated and non-methylated indirubins could be explained possibly by their differential metabolism. AhR agonists are indeed known to induce their own degradation, through the induction of cytochrome P450s. It is possible that the N1-methylated indirubins are only short-lived, and the cellular response they induce only depends on the half-life of p27^{KIP1} they have induced. In contrast, the non-methylated indirubins may be much more stable in the cell environment, as they induce much less metabolizing cytochrome P450s. Being trapped in the ATP-binding pocket of various kinases, indirubins may have long-lasting effects on cells, resulting in irreversible effects and diminished cell viability.

N1-methylated indirubins thus provide very useful compounds for arresting cell cycling. In one aspect, the present invention provides N1-methylated indirubin compounds useful for treatment of cancer, including leukemia-type cancers, breast cancer and pancreatic cancer. In one aspect, N1-methylated indirubin compounds are used as an anti-tumor agent, that is, as an agent for the inhibition of tumor growth or the remission of a tumor.

By “pharmaceutically acceptable salt” as used herein, is meant to include salts of the active compound that are prepared with nontoxic acids or bases, depending on the particular substituents found-on-the compounds-described herein. When compounds of the invention contain acidic functionalities, base addition salts can be obtained by contacting the neutral form of such compounds with a sufficient amount of the desired base. Examples of pharmaceutically acceptable base addition -salts-include sodium, potassium, calcium, ammonium, organic amino, or magnesium salt, or similar salt. When compounds of the invention contain basic functionalities, acid addition salts can be obtained by contacting the neutral form of such compounds with a sufficient amount of the desired acid. Examples of pharmaceutically acceptable acid addition salts include those derived from inorganic acids like hydrochloric, hydrobromic, nitric, carbonic, sulfuric, phosphoric, hydriodic, phosphorous acids, and the like, as well as the salts derived from relatively nontoxic organic acids like acetic, propionic, isobutyric, maleic, malonic, benzoic, succinic, suberic, fumaric, citric, tartaric, and the like, and salts of organic acids like glucuronic and so forth.

### 5.1.2. Preparation of Compounds

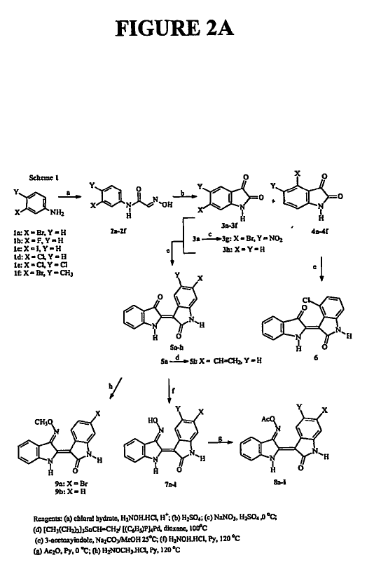

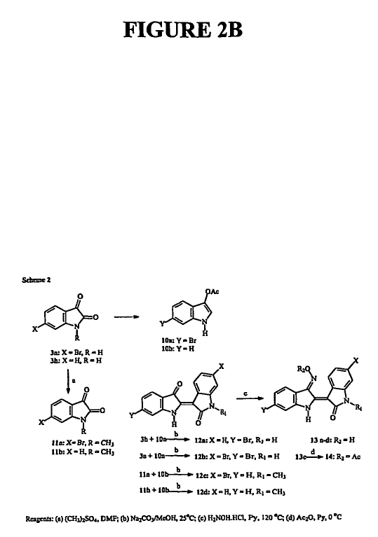

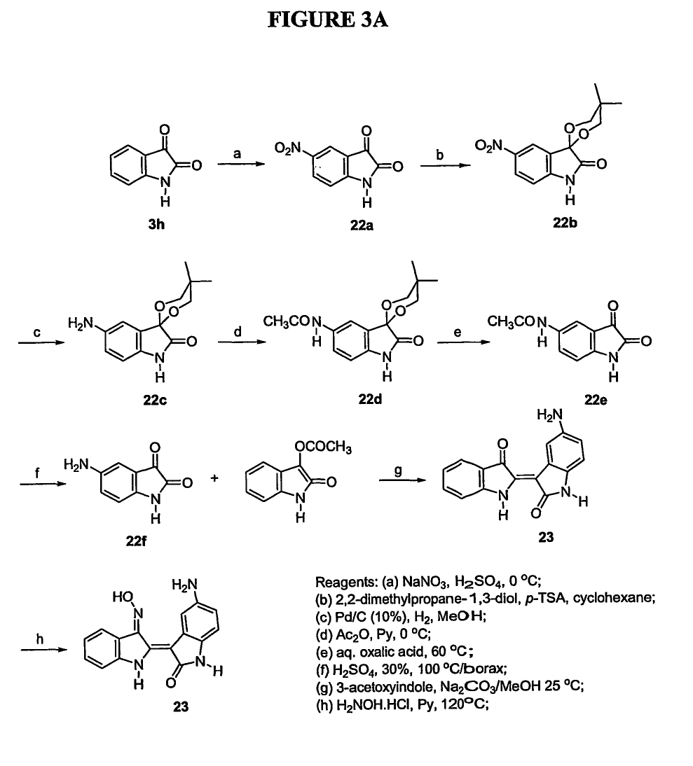

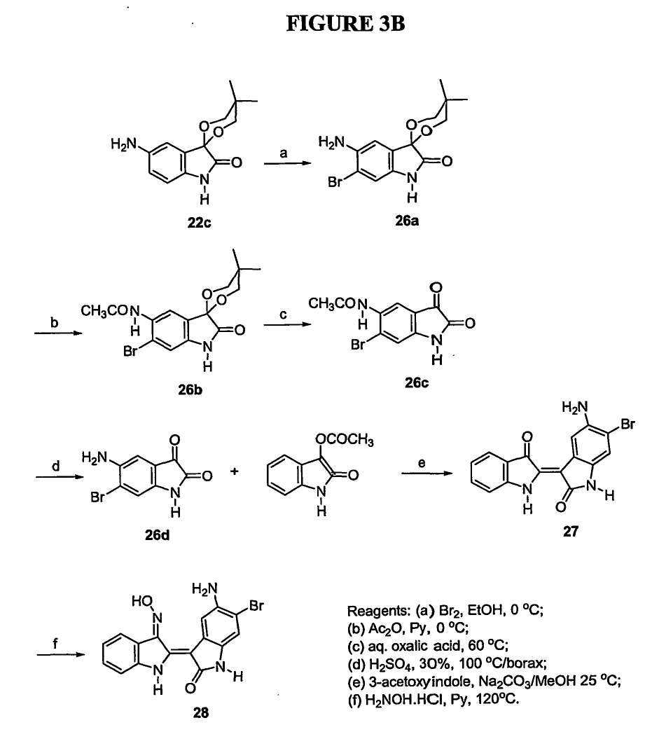

The compounds of the invention can be prepared using standard techniques known those of skill in the art. By way of example, and without limitation, the synthesis of 6-monosubstituted or 5,6-bisubstituted indirubins can be based on the dimerization reaction of an appropriately substituted isatin derivative with 3-acetoxyindol, as depicted in Scheme 1 (FIG. 2A), except of the case of 6-vinylindirubin which was prepared directly from 6-bromoindirubin employing the Still reaction. The desired isatins can be synthesized through a two-step procedure (Clark et al. (1997) Nucella lapillus. J. Soc. Dyers Colour. 113: 316-321) using the corresponding commercial mono- or bi-substituted anilines 1a-f as starting material, as described in Section 6. The acetoximes 8a-i and 14 can be prepared from the oximes 7a-i and 13c with acetic anhydride in pyridine. The temperature of the reaction was carefully kept at 0° C. to avoid bisacetylation. 5-Amino-indirubins can be prepared as described in FIGS. 3A and B.

### 5.1.3. Compositions

The present invention provides pharmaceutical compositions of the compounds of the invention disclosed hereinabove.

When employed as pharmaceuticals, the indirubin derivatives of this invention are typically administered in the form of a pharmaceutical composition. Such compositions can be prepared in a manner well known in the pharmaceutical art and comprise at least one active compound.

Generally, the compounds of this invention are administered in a pharmaceutically effective amount. The amount of the compound actually administered will typically be determined by a physician, in the light of the relevant circumstances, including the condition to be treated, the chosen route of administration, the actual compound administered, the age, weight, and response of the individual patient, the severity of the patient's symptoms, and the like.

The pharmaceutical compositions of this invention can be administered by a variety of routes including oral, rectal, transdermal, subcutaneous, intravenous, intramuscular, and intranasal. Depending on the intended route of delivery, the compounds of this invention are preferably formulated as either injectable or oral compositions or as salves, as lotions or as patches all for transdermal administration.

The compositions for oral administration can take the form of bulk liquid solutions or suspensions, or bulk powders. More commonly, however, the compositions are presented in unit dosage forms to facilitate accurate dosing. The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules or the like in the case of solid compositions. In such compositions, the indirubin-type compound is usually a minor component (from about 0.1 to about 50% by weight or preferably from about 1 to about 40% by weight) with the remainder being various vehicles or carriers and processing aids helpful for forming the desired dosing form. A “vehicle” as used herein is a substance that facilitates the use of a drug, pigment, or other material mixed with it, and the term vehicle encompasses both carriers and excipients.

Liquid forms suitable for oral administration may include a suitable aqueous or nonaqueous vehicle with buffers, suspending and dispensing agents, colorants, flavors and the like. Solid forms may include, for example, any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate; a glidant such as colloidal silicon dioxide; a sweetening agent such as sucrose or saccharin; or a flavoring agent such as peppermint, methyl salicylate, or orange flavoring.

Injectable compositions are typically based upon injectable sterile saline or phosphate-buffered saline or other injectable carriers known in the art. As before, the active compound in such compositions is typically a minor component, often being from about 0.05 to 10% by weight with the remainder being the injectable carrier and the like.

Transdermal compositions are typically formulated as a topical ointment or cream containing the active ingredient(s), generally in an amount ranging from about 0.01 to about 20% by weight, preferably from about 0.1 to about 20% by weight, preferably from about 0.1 to about 10% by weight, and more preferably from about 0.5 to about 15% by weight. When formulated as a ointment, the active ingredients will typically be combined with either a paraffinic or a water-miscible ointment base. Alternatively, the active ingredients may be formulated in a cream with, for example an oil-in-water cream base. Such transdermal formulations are well-known in the art and generally include additional ingredients to enhance the dermal penetration of stability of the active ingredients or the formulation. All such known transdermal formulations and ingredients are included within the scope of this invention.

The compounds of this invention can also be administered by a transdermal device. Accordingly, transdermal administration can be accomplished using a patch either of the reservoir or porous membrane type or of a solid matrix variety.

The above-described components for orally administrable, injectable or topically administrable compositions are merely representative. Other materials as well as processing techniques and the like are set forth in Part 8 of Remington's Pharmaceutical Sciences, 17th edition, 1985, Mack Publishing Company, Easton, Pa., which is incorporated herein by reference.

The compounds of this invention can also be administered in sustained release forms or from sustained release drug delivery systems. A description of representative sustained release materials can be found in Remington's Pharmaceutical Sciences.

The following formulation examples illustrate representative pharmaceutical compositions of this invention. The present invention, however, is not limited to the following pharmaceutical compositions.

Formulation 1—Tablets: An indirubin-type compound is admixed as a dry powder with a dry gelatin binder in an approximate 1:2 weight ratio. A minor amount of magnesium stearate is added as a lubricant. The mixture is formed into 240-270 mg tablets (80-90 mg of active amide compound per tablet) in a tablet press.

Formulation 2—Capsules: A indirubin-type compound is admixed as a dry powder with a starch diluent in an approximate 1:1 weight ratio. The mixture is filled into 250 mg capsules (125 mg of active amide compound per capsule).

Formulation 3—Liquid: Indirubin-type compound (125 mg), sucrose (1.75 g) and xanthan gum (4 mg) are blended, passed through a No. 10 mesh U.S. sieve, and then mixed with a previously made solution of microcrystalline cellulose and sodium carboxymethyl cellulose (11:89, 50 mg) in water. Sodium benzoate (10 mg), flavor, and color are diluted with water and added with stirring. Sufficient water is then added to produce a total volume of 5 mL.

Formulation 4—Injection: The indirubin-type compound is dissolved or suspended in a buffered sterile saline injectable aqueous medium to a concentration of approximately 5 mg/ml.

### 5.1.4. Compound Utility

The present C6-halogen-substituted indirubins are used as therapeutic agents for the treatment of conditions in mammals, including humans, such as (but not limited to) insulin-signaling disorders, diabetes, neurodegenerative disorders, Alzheimer's Disease, Huntington disease, and cancer. The present N1-methyl-indirubins are used as therapeutic agents for the treatment of conditions in mammals, including humans, for example (and not limited to) inhibiting tumor growth, and for treating cardiovascular disease, and cancer including leukemia and leukemia-type cancers, breast cancer and pancreatic cancer.

Injection dose levels range from about 0.1 mg/kg/hour to at least 10 mg/kg/hour, all for from about 1 to about 120 hours and especially 24 to 96 hours. A preloading bolus of from about 0.1 mg/kg to about 10 mg/kg or more may also be administered to achieve adequate steady state levels. The maximum total dose is not expected to exceed about 2 g/day for a 40 to 80 kg human patient.

For the prevention and/or treatment of long-term conditions, such as neurodegenerative and cancer conditions, the regimen for treatment usually stretches over many months or years so oral dosing is preferred for patient convenience and tolerance. With oral dosing, one to five and especially two to four and typically three oral doses per day are representative regimens. Using these dosing patterns, each dose provides from about 0.01 to about 20 mg/kg of the amide derivative, with preferred doses each providing from about 0.1 to about 10 mg/kg and especially about 1 to about 5 mg/kg.

Transdermal doses are generally selected to provide similar or lower blood levels than are achieved using injection doses.

The compounds of this invention can be administered as the sole active agent or they can be administered in combination with other agents. The combined use of 6′-bromoindirubins that selectively inhibit GSK-3 and lithium is recommended in a patient for the treatment of AD, or neurodegenerative disorder.

### 5.1.5. Methods

In one aspect, the present invention provides a method for the isolation of 6-bromoindirubin from a natural source comprising the steps described in Example 6.1.2.

In another aspect, the present invention provides a method for screening compounds comprising culturing stably transfected mouse hepatoma cells expressing XRE-driven enhanced green fluorescent protein (EGFP) fusion gene and contacting the cells with a compound to be tested.

In yet another aspect, the present invention provides a method for inhibiting GSK-3 comprising contacting a cell with a compound consisting of 6-bromoindirubin (5a) or a 6-bromoindirubin derivative or analogue. In one embodiment, the compound is selected from group of compounds consisting of 6-bromoindirubin (5a), 6,6′-dibromoindirubin (12b), 6-bromoindirubin-3′-oxime (7a), 6,6′-dibromoindirubin-3′-oxime (13b), 6-bromoindirubin-3′-methoxime (9a), and 6-bromoindirubin-3′-acetoxime (8a).

In another aspect, the present invention provides a method for preventing, treating or ameliorating type 2 diabetes or Alzheimer's disease in a mammal, comprising administering to the mammal an effective disease-treating or condition-treating amount of a pharmaceutical composition of indirubin-type compound consisting of 6-bromoindirubin (5a) or a 6-bromoindirubin derivative or analogue.

In another aspect, the present invention provides a method of effecting Gi phase arrest in a cell comprising contacting the cell with 1-methylindirubin (12d) or a 1-methylindirubin derivative or analogue.

In one aspect, the present invention provides a method for inhibiting tumor growth comprising contacting the cell with 1-methylindirubin or a 1-methylindirubin derivative or analogue.

In one aspect, the present invention provides a method for determining selective modulatory ability of an agent comprising assaying the agent in vitro for GSK-3 activity, CDK5 activity and AhR activity, wherein an agent is determined to have selective modulatory ability where its modulatory activity expressed as EC₅₀ or IC₅₀ is in the 10 micromolar or less concentration in one of the assays but greater than 100 micromolar concentration in the other two assays. In one embodiment, indirubins are evaluated for potential therapeutic applications according to their selectivity (GSK-3: type 2 diabetes, neurodegenerative disorders, Alzheimer's disease, bipolar disorder, infectious diseases such as malaria and other protozoan-derived diseases, for example; CDK5: neurodegenerative disorders and cancers, for example; AhR: for example, cancers).

In another aspect, the present invention provides a method of inhibiting tumor growth comprising administering a therapeutically effective amount of 1-methyl-indirubin or 1-methyl-6-bromoindirubin.

In one embodiment, the present invention provides a method for preventing, treating or ameliorating pancreatic cancer in a mammal, comprising administering to the mammal an effective disease-treating or condition-treating amount of a pharmaceutical composition of N1-methyldindirubin or N1-methylindirubin analogue or derivative.

Interference with the AhR signaling pathway contributes to leukemogenesis, as shown by an acute myeloblastic leukemia in which a fusion protein between TEL (Translocated ETS leukemia) and ARNT is expressed (Salomon-Nguyen et al. (2000) Proc. Natl. Acad. Sci. U.S.A. 97: 6757-6762), by the constitutive activation of AhR in adult T-cell leukemia (ATL) (Hayashibara et al. (2003) Biochem. Biophys. Res. Commun. 300: 128-134) and by the benzene-induced AHR-dependent hematotoxicity/leukemogenesis (Yoon et al. (2002) Toxicol. Sci. 70: 150-156).

In another embodiment, the present invention provides a method for preventing, treating or ameliorating leukemia in a mammal, comprising administering to the mammal an effective disease-treating or condition-treating amount of a pharmaceutical composition of N1-methyldindirubin or N1-methylindirubin analogue or derivative. In a preferable embodiment, the leukemia is adult T-cell leukemia.

In another aspect, the present invention provides a method of structural-based design using models of GSK-3 and GSK-3 inhibitors. In one embodiment, the present invention provides a method for identifying a potential modulator of GSK-3 comprising the steps of using the three-dimensional structure of GSK-3 co-crystallized with a GSK-3 inhibitor and modeling methods to identify chemical entities or fragments capable of associating with GSK-3, assembling the identified chemical entities or fragments into a single molecule to provide the structure of said potential modulator, synthesizing the potential modulator, and contacting the potential modulator with GSK-3 in the presence of a GSK-3 substrate to test the ability of the potential modulator to modulate GSK-3. In one embodiment, the GSK-3 inhibitor is 6-bromoindirubin. In another embodiment, the GSK-3 inhibitor is 6-bromoindirubin-3′-oxime.

### 6. EXAMPLES

The invention is described in reference to a number of examples presented below. The examples are intended to provide illustration of certain embodiments of the invention, and should not be construed to limit the invention in any way.

### 6.1. Example 1

### Preparation of Compounds

The following example describes the isolation of exemplary compounds from natural sources as well as the synthetic preparation of exemplary compounds.

### 6.1.1. General Chemistry Experimental Procedures

All chemicals were purchased from Aldrich Chemical Co. NMR spectra were recorded on Bruker DRX 400 and Bruker AC 200 spectrometers [¹H (400 and 200 MHz) and ¹³C (50 MHz)]; chemical shifts are expressed in ppm downfield from TMS. The ¹H-¹H and the ¹H-¹³C NMR measurements were performed using standard Bruker microprograms. CI-MS spectra were determined on a Finnigan GCQ Plus ion-trap mass spectrometer using CH₄ as the CI ionization reagent. Medium pressure liquid chromatography (“MPLC”) was performed with a Büchi model 688 apparatus on columns containing silica gel 60 Merck (20-40 μm) or using flash silica gel 60 Merck (40-63 μm), with an overpressure of 300 mbars. Thin layer chromatography (TLC) was performed on plates coated with silica gel 60 F₂₅₄ Merck, 0.25 mm. All the compounds gave satisfactory combustion analyses (C, H, N, within ±0.4% of calculated values).

### 6.1.2. Extraction and Isolation of Indirubins From Natural Sources

The marine mollusk, Hexaplex trunculus L., was collected in shallow waters in the Saronikos gulf near the island of Salamina (Greece). Voucher specimens are deposited in the collection of the Goulandris Natural History Museum. The mollusks (60 kg), after removal of the shells, were exposed to sunlight for 6 h, lyophilized and extracted with CH₂Cl₂ (3×15 L for 48 h). The CH₂Cl₂ extract (162 g) was subjected to vacuum liquid chromatography on silica gel 60H with increasing polarity mixtures of cyclohexane/CH₂Cl₂ (from 100:0 to 0:100) to afford 45 fractions of 500 ml. Fractions 34-45 were re-chromatographed with vacuum liquid chromatography on silica gel 60H with increasing polarity mixtures of cyclohexane/EtOAc (from 95:5 to 0:100) to afford 40 fractions of 300 ml. Fractions 36-39 were submitted to MPLC using a cyclohexane/EtOAc gradient (from 95:5 to 85:15), to give: indirubin (5h) (3.5 mg), 6′-bromoindirubin (12a) (5.5 mg), 6-bromoindirubin (5a) (2.8 mg) and 6,6′-dibromoindirubin (12b) (3 mg).

### 6.1.2.1. Spectral Data of Isolated Indirubins

6′-Bromoindirubin (12a): ¹H NMR (DMSO, 400 MHz, δ ppm, J in Hz) 11.00 (1H, s, N′—H), 10.90 (1H, s, N—H), 8.75 (1H, d, J=7.7 Hz, H-4), 7.64 (1H, s, H-7′), 7.59 (1H, d, J=8.1 Hz, H-4′), 7.27 (1H, t, J=7.7 Hz, H-6), 7.19 (1H, d, J=8.1 Hz, H-5′), 7.03 (1H, t, J=7.7 Hz, H-5), 6.92 (1H, d, J=7.7 Hz, H-7). ¹³C NMR (DMSO, 200 MHz, 5 ppm) 187.17 (C-3′), 170.33 (C-2), 152.76 (C-7a′), 140.71 (C-7a), 137.58 (C-2′), 130.34 (C-6′), 129.28 (C-6), 125.45 (C4′), 124.46 (C4), 123.65 (C-5′), 120.93 (C-5,3a), 117.77 (C-3a′), 115.82 (C-7′), 109.24 (C-7), 107.26 (C-3); CI-MS m/z 341, 343 (M+H)⁺. Anal. (C₁₆H₉N₂O₂Br) C, H, N.

6-Bromoindirubin (5a): ¹H NMR (DMSO, 400 MHz, δ ppm, J in Hz) 11.10 (1H, s, N′—H), 11.00 (1H, s, N—H), 8.67 (1H, d, J=8.1 Hz, H-4), 7.65 (1H, d, J=7.5 Hz, H-4′), 7.58 (1H, t, J=7.5 Hz, H-6′), 7.42 (1H, d, J=7.5 Hz, H-7′), 7.22 (1H, dd, J=8.1, 1.7 Hz, H-5), 7.04 (1H, d, J=1.7 Hz, H-7), 7.03 (1H, t, J=7.5 Hz, H-5′). ¹³C NMR (DMSO, 200 MHz, δ ppm) 188.85 (C-3′), 170.98 (C-2), 152.58 (C-7a′), 142.59 (C-7a), 138.86 (C-2′), 137.31 (C-6′), 125.99 (C-4), 124.52 (C-4′), 123.78 (C-5), 121.58 (C-3,5′), 120.86 (C-3a), 119.06 (C-3a′), 113.64 (C-7′), 112.39 (C-7), 105.42 (C-6); CI-MS m/z 341, 343 (N+H)⁺. Anal. (C₁₆H₉N₂O₂Br) C, H, N.

6,6′-Dibromoindirubin (12b): ¹H NMR (DMSO, 400 MHz, δppm, J in Hz) 11.20 (1H, s, N′—H), 11.10 (1H, s, N—H), 8.67 (1H, d, J=8.4 Hz, H-4), 7.68 (1H, d, J=1.7 Hz H-7′), 7.62 (1H, d, J=8.1 Hz, H-4′), 7.22 (1H, dd, J=8.1, 1.7 Hz, H-5′), 7.22 (1H, dd, J=8.4, 1.6 Hz, H-5), 7.05 (1H, d, J=1.6 Hz, H-7). ¹³C NMR (DMSO, 200 MHz, δ ppm) 187.60 (C-3′), 170.69 (C-2), 153.04 (C-7a′), 142.49 (C-7a), 130.99 (C-6′), 129.55 (C-2′), 126.25 (C4), 126.06 (C4′), 124.45 (C-5′), 124.08 (C-5), 121.95 (C-3), 121.04 (C-3a), 118.31 (C-3a′), 116.47 (C-7′), 112.50 (C-7), 106.01 (C-6); CI-MS m/ 419, 421, 423 (M+H)⁺. Anal. (C₁₆H-8N₂O₂Br₂) C, H, N.

### 6.1.3. Synthetic Preparation of Exemplary Compounds

The synthesis of indirubin-type compounds was based on the dimerization reaction of an appropriately substituted isatin derivative with 3-acetoxyindol, as depicted in the schemes shown in FIGS. 2A, 2B, 3A and 3B as explained in greater detail below.

Preparation of isatins (3a-f): Chloral hydrate (50 g) and Na₂SO₄ (350 g) were dissolved in water (700 mL) in a 3 L beaker and warmed to 35° C. A warm solution of the appropriate commercial aniline derivative la-f (0.276 mol) in water (200 mL) and conc. HCl (30 mL) was added (a white-precipitate of the amine sulfite was formed), followed by a warm solution of hydroxylamine hydrochloride (61 g) in water (275 mL). The mixture was stirred by hand and heated on a hot plate (a thick paste formed at 65-70° C.) at 80-90° C. for 2 h, then allowed to cool for 1 h, by which time the temperature had fallen to 50° C., and filtered. The pale cream product was washed by stirring with water (1 L) and filtered. Drying overnight at 40° C. gave the corresponding isonitrosoacetanilide 2a-f (see Scheme 1 in FIG. 2A).

Sulfuric acid (1 L) was heated in a 3 L beaker on a hot plate to 60° C. and then removed. The dry isonitrosoacetanilide 2a-f was added in portion with stirring over 30 min so that the temperature did not exceed 65° C. The mixture was then heated to 80° C. for 15 min, allowed to cool to 70° C. and cooled on ice. The solution was poured onto crushed ice (5 L) and left to stand for 1 h before filtering the orange-red precipitate. The product was washed by stirring with water (400 mL) and filtered to give a mixture of 3a-f and 4a-f. The crude product was dissolved in a solution of NaOH (20 g) in water (200 mL) at 60° C., and then acidified with acetic acid (60 mL). After standing 0.5 h and cooling to 35° C., the 4a-4f precipitate was filtered and washed with water (50 mL). The combined filtrate and washings were acidified with conc. HCL (60 mL) and, after standing for 2 h at 5° C., the 3a-f precipitate was filtered off and washed with water (50 mL). Yields: 3a: 27%, 3b: 14%, 3c: 29%, 3d: 22%, 3e: 33%, 3f: 31%, 4a: 56%, 4b: 64%, 4c: 53%, 4d: 59%, 4e: 49%, 4f: 51%.

6-Bromo-mnitroisatin (3g): To a solution of NaNO₃ (188 mg, 2.21 mmol) in concentrated H₂SO₄ (3.8 mL) was added drop wise a solution of 3a (500 mg, 2.21 mmol) in concentrated H₂SO₄ (3.2 mL) for a period of 1 h at 0° C. Then the reaction mixture was poured into ice water (25 mL), the precipitate was collected by filtration and washed with water to give 7g (91%).

6-Bromo-N-methylisatin (11a): To a solution of 3a (150 mg, 0.66 mmol) in dry acetone (30 mL) was added Na₂CO₃ (an.) (1.0 g) and dimethylsulfate (0.8 mL) under Ar and the reaction mixture was heated at 60° C. for 20 h. Then, the mixture was filtered and the filtrate was carefully evaporated using a high vacuum pump (under 40° C.). The solid residue was submitted to flash chromatography with CH₂Cl₂ to afford 11a (140 mg, 0.58 mmol, 85%) (see Scheme 2 in FIG. 2B).

N-Methylisatin (11b): This compound was prepared from isatin (3h) by a procedure analogous to that of 11a: yield 85%.

6-Bromoindirubin (5a): Methanol (18 mL) was vigorously stirred under nitrogen for 20 min and then 6-bromoisatin (3a) (100 mg, 0.44 mmol) and 3-acetoxyindol (77 mg, 0.44 mmol) were added and stirring was continued for 5 min. Anhydrous Na₂CO₃ (114 mg, 1.1 mmol) was added and the stirring was continued for 3 h. The dark precipitate was filtered and washed with aqueous methanol (1:1, 10 mL) to give 5a (135 mg, 0.39 mmol, 90%). Spectral data was identical to that of isolated 6-bromoindirubin (see Section 6.1.2.1, above).

6-Fluoroindirubin (5b): This compound was prepared from 6-fluoroisatin (3b) by a procedure analogous to that of 5a: yield 75%; ¹H NMR (DMSO, 400 MHz, δ ppm, J in Hz) 10.99 (1H, brs, N—H), 8.79 (1H, dd, J=8.7, 6.2 Hz, H-4), 7.65 (1H, d, J=7.5 Hz, H-4′), 7.58 (1H, t, J=7.5 Hz, H-6′), 7.41 (1H, d, J=7.5 Hz, H-7′), 7.02 (1H, t, J=7.5, Hz, H-5′), 6.83 (1H, td, J=8.7, 2.5 Hz, H-5), 6.71 (1H, dd, J=9.1, 2.5 Hz, H-7); CI-MS n/z 281 (M+H)⁺. Anal. (C₁₆H₉N₂O₂F) C, H, N.

6-Iodoindirubin (5c): This compound was prepared from 6-iodoisatin (3c) by a procedure analogous to that of Sa: yield 91%;¹H NMR (DMSO, 400 MHz, δ ppm, J in Hz) 11.04 (1H, s, N′—H), 10.95 (1H, brs, N—H), 8.48 (1H, d, J=8.5 Hz, H-4), 7.61 (1H, d, J=7.8 Hz, H-4′), 7.54 (1H, t, J=7.8 Hz, H-6′), 7.37 (2H, m, H-5, 7′), 7.19 (1H, s, H-7), 6.99 (1H, t, J=7.8 Hz, H-5′); CI-MS m/z 389 (M+H)⁺. Anal. (C₁₆H₉N₂O₂I) C, H, N.

6-Chloroindirubin (5d): This compound was prepared from 6-chloroisatin (3d) by a procedure analogous to that of 5a: yield 84%; ¹H NMR (DMSO, 400 MHz, δ ppm, J in Hz) 11.07 (1H, s, N′—H), 11.05 (1H, brs, N—H), 8.76 (1H, d, J=8.5 Hz, H-4), 7.65 (1H, d, J=7.2 Hz, H-4′), 7.58 (1H, t, J=7.2 Hz, H-6′), 7.43 (1H, d, J=7.2 Hz, H-7′), 7.07 (1H, dd, J=8.5, 1.7 Hz, H-5), 7.03 (1H, t, J=7.2 Hz, H-5′), 6.91 (1H, t, J=1.7 Hz, H-7); CI-MS m/z 297,299 (M+H)⁺. Anal. (C₁₆H₉N₂O₂C1) C, H, N.

5,6-Dichloroindirubin (5e): This compound was prepared from 5,6-dichloroisatin (3e) by a procedure analogous to that of 9a: yield 77%; ¹H NMR (I)MSO, 400 MHz, δ ppm, J in Hz) 11.17 (1H, s, N—H′) 8.94 (1H, s, H-4), 7.67 (1H, d, J-=7.5 Hz, H-4′), 7.60 (1H, t, J=7.5 Hz, H-6′), 7.43 (1H, d, J=7.5, Hz, H-7′), 7.08 (1H, s, H-7), 7.06 (1H, t, J=7.5 Hz, H-5′); CI-MS m/z 331, 333,335 (M+H)⁺. Anal. (C₁₆H₈N₂O₂Cl₂) C, H, N.

6-Bromo-5-methylindirubin (5f): This compound was prepared from 6-bromo-5-methylisatin (3f) by a procedure analogous to that of 5a: yield 76%; ¹H NMR (DMSO, 400 MHz, δ ppm, J in Hz) 11.06 (1H, s, N′—H), 10.94 (1H, brs, N—H) 8.73 (1H, s, H-4), 7.65 (1H, d, J=7.5 Hz, H-4′), 7.58 (1H, t, J=7.5 Hz, H-6′), 7.42 (1H, d, J=7.5, Hz, H-7′), 7.06 (1H, s, H-7), 7.03 (1H, t, J=7.5 Hz, H-5′), 2.36 (3H, s, 5-CH₃); CI-MS ?m/z 355, 357 (M+H)⁺. Anal. (C₁₇H₁₁N₂O₂Br) C, H, N.

6-Bromo-5-nitroindirubin (5g): This compound was prepared from 6-bromo-5-nitroisatin (3g) by a procedure analogous to that of 5a: yield 41%; ¹H NMR (DMSO, 400 MHz, δ ppm, J in Hz) 11.57 (1H, brs, N′—H), 11.31 (1H, s, N—H) 9.46 (1H, s, H-4), 7.70 (1H, d, J=7.5 Hz, H-4′), 7.62 (1H, t, J=7.5 Hz, H-6′), 7.45 (1H, d, J=7.5, Hz, H-7′), 7.26 (1H, s, H-7), 7.08 (1H, t, J=7.S Hz, H-5′); CI-MS m/z 386, 388 (M+H)⁺. Anal. (C₁₆H₈N₃O₄Br) C, H, N.

6-Vinylindirubin (5i): To a solution of 6-bromoindirubin (5a) (250 mg, 0.73 mmol) in dioxane (5 mL) was added tetrakis(triphenylphosphine)palladium (18 mg) and tributylvinylstanate (0.32 mL, 1.1 mmol) and the reaction mixture was heated at 100° C. for 1 h. Then the solvent was evaporated under reduced pressure and the residue was washed with cyclohexane and recrystallized with methanol to give 5i (160 mg, 76%); ¹H NMR (DMSO, 400 MHz, δ ppm, J in Hz) 11.02 (1H, s, N′—H), 8.74 (1H, d, J=7.9 Hz, H-4), 7.65 (1H, d, J=7.5 Hz, H-4′), 7.57 (1H, t, J=7.5 Hz, H-6′), 7.42 (1H, d, J=7.5 Hz H-7′), 7.15 (1H, dd, J=7.9, 1.7 Hz, H-5) 7.02 (1H, t, J=7.5 Hz H-5′), 6.99 (1H, d, J=1.7 Hz, H-7) 6.76 (1H, dd, J=17.4, 10.8 Hz, H-1″) 5.85 (1H, d, J=17.4 Hz, H-2b″) 5.30 (1H, d, J=10.8 Hz, H-2a″); CI-MS m/z 289 (M+H)⁺. Anal. (C₁₈H₁₂N₂O₂) C, H, N.

4-Chloroindirubin (6): This compound was prepared from 4-chloroisatin (4d) by a procedure analogous to that of 5a: yield 5%; ¹H NMR (DMSO, 400 MHz, δ ppm, J in Hz) 11.24 (1H, br s, N′—H), 10.95 (1H, br s, N—H), 7.64 (1H, d, J=7.2 Hz, H-4′), 7.56 (1H, t, J=7.2 Hz, H-6′), 7.35 (1H, d, J=7.2 Hz, H-7′), 7.19 (1H, t, J=7.5 Hz, H-6), 7.03 (1H, t, J=7.2 Hz, H-5′), 7.00 (1H, d, J=7.5 Hz, H-5), 6.83 (1H, d, J=7.5 Hz, H-7); CI-MS m/z 297, 299 (M+H)⁺. Anal. (C₁₆H₉N₂O₂Cl) C, H, N.

6′-Bromoindirubin (12a). This compound was prepared from isatin (3h) and 6-bromo-3-acetoxyindol (10a) by a procedure analogous to that of 5a: yield 80%. Spectral data identical to that of isolated 6′-bromoindirubin (see Section 6.1.2.1, above).

6,6′-Dibromoindirubin (12b). This compound was prepared from 6-bromoisatin (3a) and 6-bromo-3-acetoxyindol (10a) by a procedure analogous to that of 5a: yield 76%. Spectral data identical to that of isolated 6,6′-Dibromoindirubin (see Section 6.1.2.1, above).

6-Bromo-1-methylindirubin (12c). This compound was prepared from 6-bromo-N-methylisatin (11a) and 3-acetoxyindol (10b) by a procedure analogous to that of 5a: yield 49%; ¹H NMR (DMSO, 400 M, δ ppm, J in Hz) 11.13 (1H, s, N′—H), 8.70 (1H, d, J=8.2 Hz, H-4), 7.66 (1H, d, J=7.8 Hz, H-4′), 7.60 (1H, t, J=7.8 Hz, H-6′), 7.43 (1H, d, J=7.8, Hz, H-7′), 7.35 (1H, d, J=1 Hz, H-7), 7.29 (1H, dd, J=8.2, 1 Hz, H-5), 7.04 (1H, t, J=7.8, Hz, H-5′), 3.28 (3H, s, N—CH₃); CI-MS m/z 355, 357 (M+H)⁺. Anal. (C₁₇H₁₁N₂O₂Br) C, H, N.

1-Methylindirubin (12d). This compound was prepared from N-methylisatin (11b) and 3-acetoxyindol (10b) by a procedure analogous to that of 5a: yield 44%; ¹H NMR (DMSO, 400 MHz, δ ppm, J in Hz) 11.07 (1H, s, N′—H), 8.80 (1H, d, J=7.5 Hz, H-4), 7.66 (1H, d, J=7.5 Hz, H-4′), 7.59 (1H, t, J=7.5 Hz, H-6′), 7.42 (1H, d, J=7.5 Hz, H-7′), 7.35 (1H, t, J=7.5 Hz, H-6), 7.10 (1H, t, J=7.5 Hz, H-5), 7.08 (1H, d, J=7.5 Hz, H-7), 7.03 (1H, t, J=7.5 Hz, H-5′), 3.28 (3H, s, N—CH₃); Cl-MS m/z 277 (M+H)⁺. Anal. (C₁₇H₁₂N₂O₂) C, H, N.

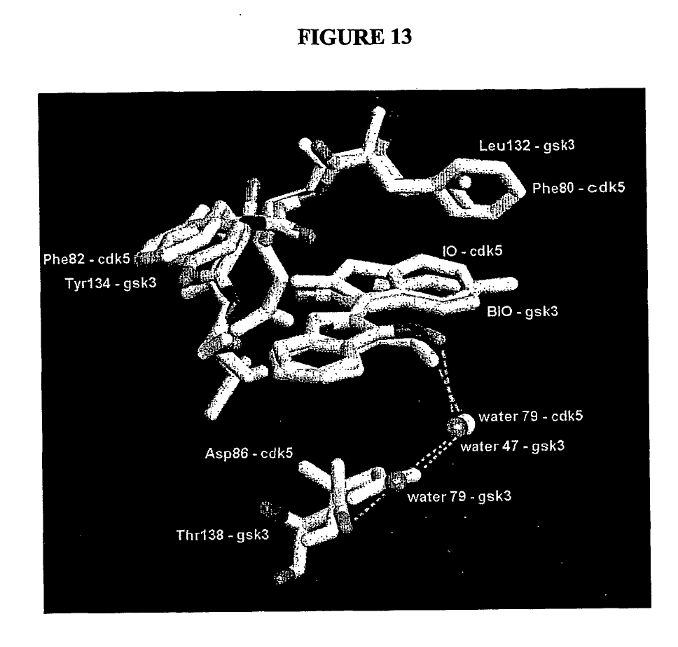

General procedure for the preparation of the oximes 7a-i (FIG. 2A) and 13a-d (FIG. 2B): The appropriate indirubin derivative 5a-i and 12a-d (1 mmol) was dissolved in pyridine (10 mL). With magnetic stirring, hydroxylamine hydrochloride (10 equiv), was added and the mixture was heated under reflux (120° C.) for 1.5 h. Then the solvent was evaporated under reduced pressure and the residue was washed with water and cyclohexane to afford quantitatively the corresponding 3′-oxime.

6-Bromoindirubin-3′-oxime (“BIO”) (7a): ¹H NMR (DMSO, 400 MHz, δ ppm, J in Hz) 13.61 (1H, br s, NOH), 11.72 (1H, s, N′—H), 10.85 (1H, s, N—H), 8.53 (1H, d, J=8.2 Hz, H-4), 8.19 (1H, d, J=7.5 Hz, H-4′), 7.39 (2H, br s, H-6′, 7′), 7.07 (1H, d, J=8.2, Hz, H-5), 7.01 (2H, br s, H-7, 5′); CI-MS m/z 356, 358 (M+H)⁺. Anal. (C₁₆H₁₀N₃O₂Br) C, H, N.

6-Fluoroindirubin-3′-oxime (7b): ¹H NMR (DMSO, 400 MHz, δ ppm, J in Hz) 13.52 (1H, s, NOH), 11.65 (1H, s, N—H), 10.86 (1H, s, N′—H) 8.65 (1H, dd, J=8.8, 5.9 Hz, H-4), 8.23 (1H, d, J=7.3 Hz, H-4′), 7.40 (2H, m, H-6′, 7′), 7.02 (1H, m, H-5′), 6.75 (1H, td, J=8.8, 2.4 Hz, H-5), 6.70 (1H, dd, J=8.8, 2.4 Hz, H-7); CI-MS m/z 296 (M+H)⁺. Anal. (C₁₆H₁₀N₃O₂F) C, H, N.

6-Iodoindirubin-3′-oxime (7c): ¹H NMR (Acetone, 400 MHz, δ ppm, J in Hz) 13.20 (1H, s, NOH), 11.70 (1H, s, N′—H), 9.98 (1H, s, N—H), 8.43 (1H, d, J=8.3 Hz, H-4), 8.28 (1H, d, J=7.5 Hz, H-4′), 7.39 (1H, t, J=7.5 Hz, H-6′), 7.30 (1H, d, J=7.5, Hz, H-7′), 7.28 (1H, d, J=1.3 Hz, H-7) 7.23 (1-H, dd, J=8.3, 1.3 Hz, H-5) 7.03 (1H, t, J=7.5 Hz, H-5′); CI-MS m/z 404 (M+H)⁺. Anal. (C₁₀H₁₀N₃O₂I) C, H, N.

6-Chloroindirubin-3′-oxime (7d): ¹H NMR (Acetone, 400 MHz, δ ppm, J in Hz) 11.76 (1H, s, N′—H), 10.10 (1H, s, N—H), 8.71 (1H, d, J=8.8 Hz, H-4), 8.37 (1H, d, J=7.5 Hz, H-4′), 7.48 (1H, t, J=7.5 Hz, H-6′), 7.38 (1H, d, J=7.5 Hz, H-7′) 7.12 (1H, t, J=7.5 Hz, H-5′), 7.04 (1H, d, J=2.2 Hz, H-7) 7.12 (1H, dd, J=8.8,2.2 Hz, H-5); CI-MS m/z 312, 314 (M+H)⁺. Anal. (C₁₆H₁₀N₃O₂Cl) C, H, N.

5,6-Dichloroindirubin-3′-oxime (7e): ¹H NMR (DMSO, 400 MHz, δ ppm, J in Hz) 13.76 (1H, brs, NOH), 11.88 (1H, s, N′—H), 10.90 (1H, s, N—H), 8.80 (1H, s, H-4), 8.25 (1H, d, J=7.5 Hz, H-4′), 7.44 (1H, d, J=7.9 Hz, H-7′), 7.39 (1H, t, J=7.9 Hz, H-6′) 7.06 (1H, t, J=7.9 Hz, H-5′) 7.03 (1H, s, H-7); Cl-MS mn/z 346, 348, 350 (M+H)⁺. Anal. (C₁₆H₉N₃O₂Cl₂) C, H, N.

6-Bromo-5-methylindirubin-3′-oxime (7f): ¹H NMR (DMSO, 400 MHz, δ ppm, J in Hz) 13.56 (1H, brs, NOH), 11.74 (1H, s, N′—H), 10.69 (1H, s, N—H), 8.62 (1H, s, H-4), 8.22 (1H, d, J=7.9 Hz, H-4′), 7.39 (2H, m, H-6′, 7′), 7.03 (1H, s, H-7),7.04 (1H, dd, J=8.1, 2.1 Hz, H-5′), 2.37 (3H, s, 5-CH₃); CI-MS m/z 370, 372 (M+H)⁺. Anal. (C₁₇H₁₃N₃O₂Br) C, H, N.

6-Bromo-5-nitroindirubin-3′-oxime (7g): ¹H NMR (DMSO, 400 MHz, δ ppm, J in Hz) 13.90 (1H, brs, NOH), 11.91 (1H, s, N′—H), 11.33 (1H, s, N—H), 9.20 (1H, s, H-4), 8.24 (1H, d, J=7.5 Hz, H-4′), 7.50 (1H, d, J=7.5 Hz, H-7′), 7.43 (1H, t, J=7.5 Hz, H-6′) 7.23 (1H, s, H-7), 7.10 (1H, t, J=7.5 Hz, H-5′); Cl-MS m/z 401, 403 (M+H)⁺. Anal. (C₁₆H₉N₄O₄Br) C, H, N.

6-Vinylindirubin-3′-oxime (7i): ¹H NMR (DMSO, 400 MHz, δ ppm, J in Hz) 13.57 (1H, brs, NOH), 11.72 (1H, s, N′—H), 10.76 (1H, s, N—H), 8.60 (1H, d, J=8.3 Hz, H-4), 8.23 (1H, d, J=7.3 Hz, H-4′), 7.41 (2H, m, H-6′, 7′), 7.03 (3H, m, H-5,5′, 7), 6.74 (1H, dd, J=17.6, 10.8 Hz, H-1″) 5.77 (1H, d, J=17.6 Hz, H-2b″) 5.22 (1H, d, J=10.8 Hz, H-2a″); CI-MS m/z 304 (+H)⁺. Anal. (C₁₈H₁₃N₃O₂) C, H, N.

6′-Bromo-indirubin-3′-oxime (13a): ¹H NMR (DMSO, 400 MHz, δ ppm, J in Hz) 13.75 (1H, br s, NOH), 11.70 (1H, s, N′—H), 10.72 (1H, s, N—H), 8.61 (1H, d, J=7.9 Hz, H-4), 8.12 (1H, d, J=8.3 Hz, H-4′), 7.63 (1H, d, J=1.2 Hz, H-7′), 7.19 (1H, dd, J=1.2, 8.3 Hz, H-5′), 7.14 (1H, d, J=7.9 Hz, H-6), 6.95 (1H, t, J=7.9 Hz, H-5), 6.91 (1H, d, J=7.9 Hz, H-7); CI-MS 7 m/z 356, 358 (M+H)⁺. Anal. (C₁₆H₁₀N₃O₂Br) C, H, N.

6,6′-dibromo-indirubin-3′-oxime (13b): ¹H NMR (DMSO, 400 MHz, δ ppm, J in Hz) 13.84 (1H, br s, NOH), 11.70 (1H, s, N′—H), 10.85 (1H, s, N—H), 8.51 (1H, d, J=8.7 Hz, H-4), 8.10 (1H, d, J=8.3 Hz, H-4′), 7.63 (1H, s, H-7′), 7.20 (1H, d, J=8.3 Hz, H-5′), 7.08 (1H, d, J=8.7 Hz, H-5), 7.06 (1H, s, H-7); CI-MS m/z 434, 436,438 (M+H)⁺. Anal. (C₁₆H₉N₃O₂Br₂) C, H, N.

6-Bromo-1-methylindirubin-3′-oxime (13c): ¹H NMR (DMSO, 400 MHz, δ ppm, J in Hz) 13.69 (1H, brs, NOH), 11.78 (1H, s, N′—H), 8.61 (1H, d, J=8.1 Hz, H-4), 8.23 (1H, d, J=7.3 Hz, H-4′), 7.44 (2H, m, H-6′, 7′), 7.31 (1H, s, H-7), 7.17 (1H, d, J=8.1 Hz, H-5), 7.07 (1H, br s, H-5′), 3.32 (3H, s, N-CH₃); CI-MS m/z 370, 372 (M+H)⁺. Anal. (C₁₇H₁₂N₃O₂Br) C, H, N.

1-Methylindirubin-3′-oxime (13d): ¹H NMR (DMSO, 400 MHz, δ ppm, J in Hz) 13.56 (1H, br s, NOH), 11.74 (1H, s, N′—H), 8.69 (1H, d, J-=7.8 Hz, H-4), 8.23 (1H, d, J=7.5 Hz, H-4′), 7.41 (2H, m, H-6′, 7′), 7.23 (1H, t, J=7.8, Hz, H-6), 7.04 (3H, m, H-5′, 5, 7), 3.31 (3H, s, N—CH₃); CI-MS m/z 292 (M+H)⁺. Anal. (C₁₇H₁₃N₃O₂) C, H, N.

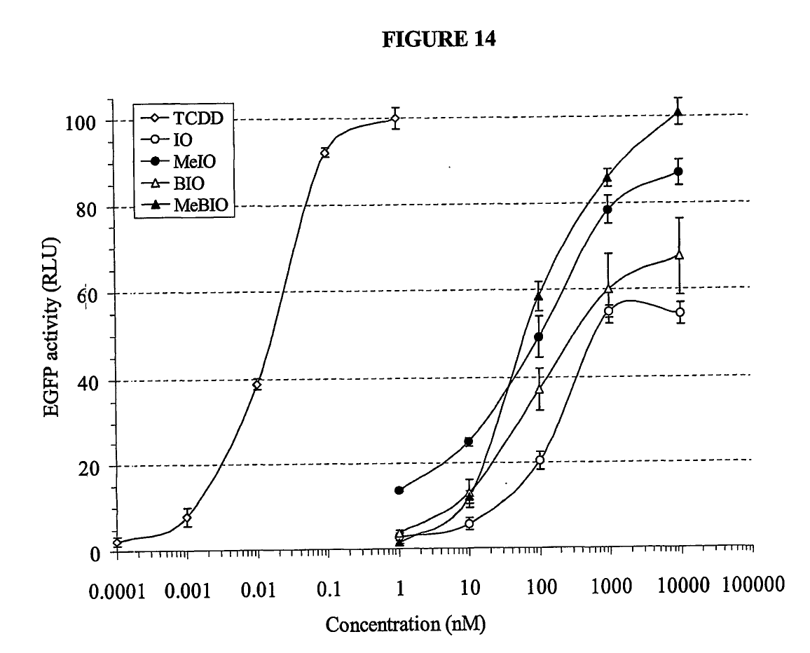

General procedure for the preparation of the acetoximes 8a-i (FIG. 2A) and 14 (FIG. 2B): The appropriate indirubin-3′-oxime derivative 7a-i and 13c (0.2 mmol) was dissolved in pyridine (10 mL). Ac₂O was added (0.5 mL) and the mixture was stirred for 30 min at 0° C. Then water (1 mL) was added and the solvents were evaporated under reduced pressure. The residue was washed with water and cyclohexane to afford quantitatively the corresponding 3′-acetoxime.

6-Bromoindirubin-3′-acetoxime (8a): ¹H NMR (DMSO, 400 MHz, δ ppm, J in Hz) 11.60 (1H, s, N′—H), 11.01 (1H, s, N—H), 9.02 (1H, d, J=8.5 Hz, H-4), 8.24 (1H, d, J=7.8, H-4′), 7.52 (1H, d, J=7.8, H-7′), 7.49 (1H, t, J=7.8 Hz, H-6′), 7.10 (1H, t, J=7.8 Hz, H-5′), 7.09 (1H, d, J=8.5 Hz, H-5), 7.04 (1H, s, H-7), 2.47 (3H, s, OCOCH₃); CI-MS m/z 397, 399 (M+H)⁺. Anal. (C₁₈H₁₂N₃O₃Br) C, H, N.

6-Fluoroindirubin-3′-acetoxime (8b): ¹H NMR (DMSO, 400 MHz, δ ppm, J in Hz) 11.50 (1H, s, N′—H), 11.03 (1H, s, N—H), 9.12 (1H, dd, J=8.7, 5.8 Hz, H-4), 8.25 (1H, d, J=7.5, H-4′), 7.52 (1H, t, J=7.5, H-6′), 7.46 (1H, d, J=7.5 Hz, H-7′), 7.08 (1H, t, J=7.5 Hz, H-5′), 6.73 (2H, m, H-5, 7), 2.46 (3H, s, OCOCH₃); CI-MS m/z 338 (M+H)⁺. Anal. (C₁₈H₁₂N₃O₃F) C, H, N.

6-Iodoindirubin-3′-acetoxime (8c): ¹H NMR (DMSO, 400 MHz, δ ppm, J in Hz) 11.57 (IH, s, N′—H), 10.92 (1H, s, N—H), 8.83 (1H, d, J=8.3 Hz, H-4), 8.21 (1H, d, J=7.9, H-4′), 7.50 (1H, d, J=7.9, H-7′), 7.45 (1H, t, J=7.9 Hz, H-6′), 7.25 (1H, d, J=8.3 Hz, H-5), 7.18 (1H, s, H-7), 7.06 (1H, t, J=7.9 Hz, H-5′), 2.47 (3H, s, OCOCH₃); CI-MS m/z 446 (M+H)⁺. Anal. (C₁₈H₁₂N₃O₃I) C, H, N.

6-Chloroindirubin-3′-acetoxime (8d): ¹H NMR (DMSO, 400 MHz, δ ppm, J in Hz) 11.59 (1H, s, N′—H), 11.02 (1H, s, N—H), 9.09 (1H, d, J=8.2 Hz, H-4), 8.25 (1H, d, J=7.8 Hz, H-4′), 7.50 (2H, m, H-6′, 7′), 7.10 (1H, t, J=7.8 Hz, H-5′), 6.96 (1H, dd, J=2.3, 8.2 Hz, H-5), 6.91 (1H, d, J=2.3 Hz, H-7), 2.47 (3H, s, OCOCHH₃); CI-MS m/z 354, 356 (M+H)⁺. Anal. (C₁₈H₁₂N₃O₃Cl) C, H, N.

5,6Dichloroindirubin-3′-acetoxime (8e): ¹H NMR (DMSO, 400 MHz, δ ppm, J in Hz) 11.65 (1H, s, N′—H), 11.08 (1H, s, N—H), 9.33 (1H, s, H-4), 8.24 (1H, d, J=7.4 Hz, H-4′), 7.52 (2H, m, H-6′, 7′), 7.12 (1H, t, J=7.4 Hz, H-5′) 7.04 (1H, s, H-7) 2.48 (3H, s, OCOCH₃); CI-MS m/z 388, 390,392 (M+H)⁺. Anal. (C₁₈H₁₁N₃O₃Cl₂) C, H, N.

6-Bromo-5-methylindirubin-3′-acetoxime (8f): ¹H NMR (DMSO, 400 MHz, δ ppm, J in Hz) 11.56 (1H, s, N′—H), 10.87 (1H, s, N—H), 9.16 (1H, s, H-4), 8.26 (1H, d, J=7.8 Hz, H-4′), 7.50 (2H, m, H-6′, 7′), 7.10 (1H, t, J=7.8 Hz, H-5′), 7.05 (1H, s, H-7), 2.48 (3H, s, OCOCH₃), 2.39 (3H, s, 5—CH₃); CI-MS m/z 412, 414 (M+H)⁺. Anal. (C₁₉H₁₄N₃O₃Br) C, H, N.

6-Bromo-5-nitroindirubin-3′-acetoxime (8g): ¹H NMR (DMSO, 400 MHz, δ ppm, J in Hz) 11.73 (1H, s, N′—H), 11.41 (1H, s, N—H), 9.56 (1H, s, H-4), 8.22 (1H, d, J=7.8 Hz, H-4′), 7.53 (2H, m, H-6′, 7′), 7.21 (1H, s, H-7), 7.13 (1H, t, J=7.8 Hz, H-5′) 2.46 (3H, s, OCOCH₃); CI-MS m/z 443, 445 (M+H)⁺. Anal. (C₁₈H₁₁N₄O₅Br) C, H, N.

Indirubin-3′-acetoxime (8h): ¹H NMR (DMSO, 400 MHz, δ ppm, J in Hz) 11.59 (1H, s, N′—H), 10.87 (1H, s, N—H), 9.08 (1H, d, J=7.8 Hz, H-4), 8.26 (1H, d, J=7.8 Hz, H-4′), 7.52 (1H, t, J=7.8 Hz, H-6′), 7.46 (1H, t, J=7.8 Hz, H-7′) 7.20 (1H, t, J=7.8 Hz, H-6),7.08 (1H, t, J=7.8 Hz, H-5),6.97 (1H, t, J=7.8 Hz, H-5′), 6.90 (1H, d, J=7.8 Hz, H-7), 2.47 (3H, s, OCOCH₃); CI-MS m/z 320 (M+H)⁺. Anal. (C₁₈H₁₃N₃O₃) C, H, N.

6-vinylindirubin-3′-acetoxime (8i): ¹H NMR (DMSO, 400 MHz, δ ppm, J in Hz) 11.57 (1H, s, N′—H), 10.90 (1H, s, N—H), 9.05 (1H, d, J=8.2 Hz, H-4), 8.25 (1H, d, J=7.4 Hz, H-4′), 7.49 (2H, m, H-6′, 7′), 7.08 (2H, m, H-5, 5′) 6.99 (1H, s, H-7), 6.76 (1H, dd, J=17.6, 11.3 Hz, H-1″), 5.81 (1H, d, J=17.6 Hz, H-2b″), 5.25 (1H, d, J=10.9 Hz, H-2a″), 2.47 (3H, s, OCOCH₃); CI-MS m/z 346 (M+H)⁺. Anal. (C₂₀H₁₅N₃O₃) C, H, N.

6-bromo-1-methylindirubin-3′-acetoxime (14): ¹H NMR (DMSO, 400 MHz, δ ppm, J in Hz) 11.63 (1H, s, N′—H), 9.06 (1H, d, J=8.3 Hz, H-4), 8.25 (1H, d, J=7.4 Hz, H-4′), 7.51 (2H, m, H-6′, 7′), 7.34 (1H, d, J=1.7 Hz, H-7), 7.16 (1H, dd, J=1.7, 8.3 Hz, H-5), 7.11 (1H, t, J=7.4 Hz, H-5′), 3.31 (3H, s, N-CH₃), 2.48 (3H, s, OCOCH₃); CI-MS m/z 412, 414 (M+H)⁺. Anal. (C₁₉H₁₄N₃O₃Br) C, H, N.

6-Bromoindirubin-3′-methoxime (9a): To a solution of 6-bromoindirubin (5a) (26 mg, 0.076 mmol) in pyridine (2 mL) was added methoxylamine hydrochloride (30 mg) and the mixture was heated under reflux (120° C.) for 12 h. Then the solvent was evaporated under reduced pressure and the residue was washed with water and diethylether to afford 9a (16 mg, 0.043 mmol, 57%). ¹H NMR (DMSO, 400 MHz, δ ppm, J in Hz) 11.70 (1H, s, N′—H), 10.92 (1H, s, N—H), 8.54 (1H, d, J=8.5 Hz, H-4), 8.11 (1H, d, J=7.8, H-4′), 7.43 (2H, br s, H-6′, 7′), 7.17 (1H, d, J=8.5 Hz, H-5), 7.04 (2H, br s, H-7, 5′), 4.39 (3H, s, OCH₃); CI-MS m/z 371, 369 +H)⁺. Anal. (C₁₇H₁₂N₃O₂Br) C, H, N.

Indirubin-3′-methoxime (9b): This compound was prepared from indirubin (5h) by a procedure analogous to that of 9a: yield 63%;¹H NMR (DMSO, 400 MHz, δ ppm, J in Hz) 11.69 (1H, s, N′—H), 10.78 (1H, s, N—H), 8.63 (1H, d, J=7.8 Hz, H-4), 8.12 (1H, d, J=7.4 Hz, H-4′), 7.43 (2H, m, H-6′, 7′), 7.16 (1H, t, J=7.8, Hz, H-6), 7.01 (1H, m, H-5, 5′), 6.90 (1H, d, J=7.8 Hz, H-7), 4.38 (3H, s, OCH₃); CI-MS m/z 292 (M+H)⁺. Anal. (C₁₇H₁₃N₃O₂) C, H, N.

FIG. 3A depicts a scheme for the synthesis of 5-amino-indirubin (23) and 5-amino-3′-oxime-indirubin (24) as explained below. The starting material, isatin (3h) can be prepared as described above.

5-Nitroisatin (22a). A solution of NaNO₃ (5.78 g, 0.068 mol) in concentrated H₂SO₄ (100 mL) was added drop wise, with stirring, to a solution of isatin 3h (10.0 g, 0.068 mol) in concentrated H₂SO₄ (120 mL) for a period of 1 h at 0° C. Then the reaction mixture was poured into ice water (750 mL), the precipitate was collected by filtration and washed with water to give 22a. Yield 11.88 g, 91%.

5-Nitroisatin Ketal (22b). A mixture of 5-nitroisatin 22a (5.0 g, 26.0 mmol), 2,2′-dimethylpropane-1,3-diol (2.69 g, 25.9 mmol) and a catalytic amount of p-TSA was suspended in cyclohexane (35 mL) and refluxed with azeotropic removal of water using Dean-Stark apparatus, overnight. The reaction mixture was cooled to room temperature and the solid that separated was filtered, washed with dilute aqueous sodium bicarbonate, water and then air dried to give 22b. Yield 6.5g, 90%.

5-Aminoisatin Ketal (22c). The 5-nitroisatin ketal 22b (3.0 g, 10.8 mmol) in 25 ml of methanol was stirred with 10% Pd/C (450 mg) under a H₂ atmosphere for a period of 5 h at room temperature. The mixture was then filtered with Celite to remove the catalyst and the methanol was concentrated to give 22c. Yield 2.57 g, 96%.

5-Acetamidoisatin Ketal (22d). The 5-aminoisatin Ketal 22c (2.37 g, 9.54 mmol) was dissolved in pyridine (10 mL). Ac₂O was added (902 μL, 9.54 mmol) and the mixture was stirred overnight at 0° C. Then the solvent was concentrated to give 22d.

Yield 2.63 g, 95%.

5-Acetamidoisatin (22e). 5-Acetamidoisatin Ketal 22d (2.4 g, 8.27 mmol) stirred with saturated oxalic acid (50 mL) solution at 60° C. overnight and cooled. The mixture was filtered and washed with water to give 22e. Yield 1.2 g, 70%.

5-Aminoisatin (22f). 5-Acetamidoisatin 22e (1.2 mg, 5.88 mmol) was dissolved in 25% sulfuric acid (23 mL) and heated under reflux with magnetic stirring for 1.5 h. After cooling, by stirring, to room temperature, the precipitate was filtered and washed with aceton to give 5-acetamidoisatin sulfate (1.4 g). The dry sulfate was dissolved in warm water (50 mL) and the mixture heated to 100° C. Borax (220 mg) was added and after cooling, a dark purple precipitate was filtered and washed with water to give 22f.

Yield 200 mg, 21%.

5-Aminoindirubin (23). Methanol (23 mL) was vigorously stirred under nitrogen for 20 min and then 5-aminoisatin 22f (95 mg, 0.58 mmol) and 3-acetoxyindol (98 mg, 0.56 mmol) were added and stirring was continued for 5 min. Anhydrous Na₂CO₃ (140 mg, 1.35 mmol) was added and the stirring was continued for 3 h. The dark precipitate was filtered and washed with aqueous methanol (1:1, 30 mL) to give 23 (140 mg, 90%). ¹H NMR (DMSO, 400 MHz, δ ppm, J in Hz) 10.95 (1H, s, N′—H), 10.46 (1H, brs, N—H) 8.15 (1H, d, J=1.8 Hz, H-4), 7.63 (1H, d, J=7.5 Hz, H-4′), 7.56 (1H, t, J=7.5 Hz, H-6′), 7.40 (1H, d, J=7.5, Hz, H-7′), 7.00 (1H, t, J=7.5 Hz, H-5′), 6.59 (1H, d, J=8.0 Hz, H-7), 6.52 (1H, dd, J=1.8, 8.0 Hz, H-6), 4.74 (2H, s, 5—NH₂).

5-Amino-3′-oxime indirubin (24). The 5-aminoindirubin 23 (50 mg, 0.18 mmol) was dissolved in pyridine (5 mL). With magnetic stirring, hydroxylamine hydrochloride (10 equiv), was added and the mixture was heated under reflux (120° C.) for 1.5 h. Then the solvent was evaporated under reduced pressure and the residue was washed with water to afford quantitatively the corresponding 3′-oxime 24. Yield 100%. ¹H NMR (DMSO, 400 MHz, δ ppm, J in Hz) 13.35 (1H, brs, NOH), 11.68 (1H, s, N′—H), 10.28 (1H, s, N—H), 8.20 (1H, d, J=6.6 Hz, H-4′), 8.01 (1H, s, H-4), 7.35 (2H, m, H-6′, 7′), 6.99 (1H, t, J=6.6 Hz, H-5′), 6.58 (1H, d, J=7.7 Hz, H-7), 6.43 (1H, d, J=7.7 Hz, H-6), 4.26 (2H, brs, 5—NH₂).

FIG. 3B depicts a scheme for the synthesis of 6-bromo-5-amino-indirubin (27) and-6-bromo-5-amino-3′-oxime indirubin (28) beginning with 4aminoisatin ketal (22c) as explained below.

6-Bromo-5-aminoisatin Ketal (26a). Aminoisatinketal 22c (2.0 g, 8.06 mmol) in 200 ml absolute ethanol was cooled to 0° C., a solution of bromine in chloroform (0.5 ml Br₂ dissolved in 500 ml CHCl₃) (410 mL, 8.06 mmol) was added slowly and the reaction mixture was stirred for 3 h. The solvent was neutralized by using NaHCO₃, filtered and concentrated. The solid residue was then purified by column chromatography over silica gel to give 5. Yield 1.2 g, 45%.

6-Bromo-5-acetamidoisatin Ketal (26b). The 6-Bromo-5-aminoisatin Ketal 26a (1.08 g, 3.30 mmol) was dissolved in pyridine (10 mL). Ac₂O was added (311 μL, 3.30 mmol) and the mixture was stirred overnight at 0° C. Then the solvent was concentrated to give 26b. Yield 1.15 g, 94%.

6-Bromo-5-acetamidoisatin (26c). 6-Bromo-5-acetamidoisatin Ketal 26b (1.1 g, 2.98 mmol) stirred with saturated oxalic acid (30 mL) solution at 60° C. overnight and cooled. The mixture was filtered and washed with water to give 26c. Yield 690 mg, 81%.

6-Bromo-5-aminoisatin (26d). 6-Bromo-5-acetamidoisatin 26c (680 mg, 2.40 mmol) was dissolved in 25% sulfuric acid (9.5 mL) and heated under reflux with magnetic stirring for 1.5 h. After cooling, by stirring, at room temperature, the precipitate was filtered and washed with acetone to give 6-Bromo-5-acetamidoisatin sulfate (645 mg). The dry sulfate was dissolved in warm water and the mixture heated to 100° C. Borax (520 mg) was added and after cooling, a dark purple precipitate was filtered and washed with water to give 26d. Yield 500 mg, 86%.

6-Bromo-5-aminoindirubin (27). Methanol (80 mL) was vigorously stirred under nitrogen for 20 min and then 6-bromo-5-aminoisatin 26d (500 mg, 2.07 mmol) and 3-acetoxyindol (350 mg, 2.0 mmol) were added and stirring was continued for 5 min. Anhydrous Na₂CO₃ (450 mg, 4.34 mmol) was added and the stirring was continued for 3 h. The dark precipitate was filtered and washed with aqueous methanol (1:1, 100 mL) to give 27 (650 mg, 90%). ¹H NMR (DMSO, 400 MHz, δ ppm, J in Hz) 11.01 (1H, s, N′—H), 10.58 (1H, brs, N—H) 8.38 (1H, s, H-4), 7.65 (1H, d, J=7.5 Hz, H-4′), 7.58 (1H, t, J=7.5 Hz, H-6′), 7.42 (1H, d, J=7.5, Hz, H-7′), 7.02 (1H, t, J=7.5 Hz, H-5′), 6.86 (1H, s, H-7), 4.98 (2H, s, 5—NH₂).

6-Bromo-5-amino-3′-oxime indirubin (28). The 6-Bromo-5-aminoindirubin 27 (50 mg, 0.14 mmol) was dissolved in pyridine (5 mL). With magnetic stirring, hydroxylamine hydrochloride (10 equiv), was added and the mixture was heated under reflux (120° C.) for 1.5 h. Then the solvent was evaporated under reduced pressure and the residue was washed with water to afford quantitatively the corresponding 3′-oxime 28. Yield 100%. ¹H NMR (DMSO, 400 MHz, δ ppm, J in Hz) 13.32 (1H, brs, NOH), 11.65 (1H, s, N′—H), 10.41 (1H, s, N—H), 8.28 (1H, s, H-4), 8.20 (1H, d, J=7.0 Hz, H-4′), 7.39 (2H, m, H-6′, 7′), 7.03 (1H, brs, H-5′), 6.84 (1H, s, H-7), 4.39 (2H, brs, 5—NH₂).

### 6.2. Example 2

### Materials and Methods for Biological Testing

Indirubin analogues were tested for their abilities to modulate protein kinase activity using CDK1/cyclin B, CDK5/p25 and GSK-3o/P as exemplary kinases, and for their abilities to activate AhR-dependent transcription.

### 6.2.1. Materials for Biochemical Assays

Biochemical Reagents including sodium ortho-vanadate, EGTA, EDTA, Mops, β-glycerophosphate, phenylphosphate, sodium fluoride, dithiothreitol (DTT), glutathione-agarose, glutathione, bovine serum albumin (BSA), nitrophenylphosphate, leupeptin, aprotinin, pepstatin, soybean trypsin inhibitor, benzamidine, histone HI (type III-S) were obtained from Sigma Chemicals. [γ-³³P]-ATP was obtained from Amersham. The GS-1 peptide (YRRAAVPPSPSLSRHSSPHQSpEDEEE) (SEQ ID NO: 1) was synthesized by the Peptide Synthesis Unit, Institute of Biomolecular Sciences, University of Southampton, Southampton SO16 7PX, U.K. 2,3,7,8-tetrachlorodibenzo-p-dioxin (TCDD) was a kind gift from Dr. Steve Safe (Veterinary Physiology and Pharmacology, Texas A&M University, College Station, Tex. 77843, USA).

Buffers were prepared as follows: Homogenization Buffer: 60 mM β-glycerophosphate, 15 mM p-nitrophenylphosphate, 25 mM Mops (pH 7.2), 15 mM EGTA, 15 mM MgCl₂, 1 mM DTT, 1 mM sodium vanadate, 1 mM NaF, 1 mM phenylphosphate, 10 μg leupeptin/mL, 10 μg aprotinin/mL, 10 μg soybean trypsin inhibitor/mL and 100 μM benzamidine. BufferA: 10 MM MgCl₂, 1 mM EGTA, 1 mM DTT, 25 mM Tris-HCl pH 7.5, 50 μg heparin/mL. Buffer C: homogenization buffer but 5 mM EGTA, no NaF and no protease inhibitors.

### 6.2.2. Kinase Preparations and Assays

Kinases activities were assayed in Buffer A or C (unless otherwise stated), at 30° C., at a final ATP concentration of 15 μM. Blank values were subtracted and activities calculated as pmoles of phosphate incorporated for a 10 min. incubation. The activities are usually expressed in % of the maximal activity, i.e., in the absence of inhibitors. Controls were performed with appropriate dilutions of dimethylsulfoxide. In a few cases phosphorylation of the substrate was assessed by autoradiography after SDS-PAGE.

GSK-3α/β was purified from porcine brain by affinity chromatography onαimmobilized axin (Primot et al. (2000) Protein Expr. Purif 20: 394-404). It was assayed, following a 1/100 dilution in 1 mg BSA/mL, 10 mM DTT, with 5 μL 40 μM GS-1 peptide as a substrate, in buffer A, in the presence of 15 μM [γ-33P] ATP (3,000 Ci/mmol; 1 mCi/mL) in a final volume of 30 μL. After 30 min. incubation at 30° C., 25 μl aliquots of supernatant were spotted onto 2.5×3 cm pieces of Whatman P81 phosphocellulose paper, and, 20 sec. later, the filters were washed five times (for at least 5 min. each time) in a solution of 10 mL phosphoric acid/liter of water. The wet filters were counted in the presence of 1 mL ACS (Amersham) scintillation fluid.

CDK1/cyclin B was extracted in homogenization buffer from M phase starfish (Marthasterias glacialis) oocytes and purified by affinity chromatography on p9^{CKShs1}-sepharose beads, from which it was eluted by free p9^{CKShs1} as previously described (Borgne & Meijer (1996) J. Biol. Chem. 271: 27847-27854). The kinase activity was assayed in buffer C, with 1 mg histone H1/mL, in the presence of 15 μM [γ-³²P] ATP (3,000 Ci/mmol; 1 mCi/mL) in a final volume of 30 μL. After 10 min. incubation at 30° C., 25 μl aliquots of supernatant were spotted onto P81 phosphocellulose papers and treated as described above.

CDK5/p25 was reconstituted by mixing equal amounts of recombinant mammalian CDK5 and p25 expressed in E. coli as GST (Glutathione-S-transferase) fusion proteins and purified by affinity chromatography on glutathione-agarose (vectors kindly provided by Dr. J. H. Wang) (p25 is a truncated version of p35, the 35 kDa CDK5 activator). Its activity was assayed in buffer C as described for CDK1/cyclin B.

Other kinases were expressed, purified and assayed as described previously (Neijer et al. (2000) Chem. & Biol. 7: 51-63).

### 6.2.3. AhR Assays

Yeast AhR reporter assay: The Saccharomyces cerevisiae strain used for the yeast AhR reporter assay was YCM3, derived from YPH-499 (mat a, ade2-101, his3 Δ200, leu2-1, lys2-801, trp-63, ura3-52) (Miller (1997) J. Biol. Chem., 272: 32824-32829; Adachi et al. (2001) J. Biol. Chem. 276: 31475-31478), kindly provided by Dr. C. A. Miller III (Department of Environmental Health Sciences and Tulane-Xavier Center for Bioenvirommental Research, Tulane University School of Public Health and Tropical Medicine, New Orleans, La. 70112, USA). Standard yeast growth conditions and genetic manipulations were previously described (Guthrie & Fink (1991) Guide to Yeast Genetics and Molecular Biology (Abelson J N and Simon M I (eds). Academic Press Inc.: San Diego, Calif., U.S.A)). Yeast cells expressing both AhR and ARNT from the inducible GAL promoter were grown to early log phase at 25° C. in selective media containing raffinose (2% final concentration), at which time galactose (2%) was added for 3 hr. Various concentrations of indirubins (in DMSO) were then added and the cells were grown for an additional three hours and β-galactosidase activity was determined (adapted from Miller (1999) Toxicol Appl. Pharmacol. 160: 297-303). Briefly, 1 ml of cell culture was centrifuged and cells were washed and resuspended in 150 μl of Z-buffer (60 mM Na₂HPO₄, 40 mM NaH₂PO₄, 1 MM MgSO₄, 10 mM KCl, 40 mM β-mercaptoethanol). After addition of 50 μl chloroform and 10 μl of 0.1% SDS, the mix was vortexed for 15 seconds and the reaction was started by addition of 700 μl of o-nitrophenol-D-galactopyranoside (1 mg/ml solution in Z-buffer) and further incubated for 10 min. at 30° C. The reaction was then stopped by addition of 500 μl of 1 M Na₂CO₃. β-galactosidase (referred to as lacZ units) was measured at 420 nm and calculated according to the following formula: absorbance at 420 nm X 1000/(absorbance at 600 nm X ml of cell suspension added X min of reaction time).

AhR-dependent green fluorescent protein (EGFP) expression assay in a stably transfected mouse hepatoma cell line: Mouse hepatoma (H1G1.1c3) cells containing a stably transfected plasmid expressing XRE-driven enhanced green fluorescent protein (EGFP) fusion gene were maintained in selective media (OMEM containing 10% fetal bovine serum and 968 mg/L G418). XREs are high-affinity binding sites for AhR/ARNT transcription factor complex and they mediate ligand- and AhR-dependent transcriptional activation. (Nagy et al. (2002) Toxicol Sci. 65: 200-210; Nagy et al. (2002) Biochem. 41: 861-868). Cells were plated into black, clear-bottomed 96-well tissue culture dishes (Corning, San Mateo, CA) at 75,000 cells per well and allowed to attach for 24 hrs. Selective media was then replaced with 100 μL of media (lacking G418) containing the DMSO (1% final solvent concentration) or the test chemical at the indicated concentration. EGFP levels were measured in living cells at 24 hours using a Tecan Genios microplate fluorometer with an excitation wavelength of 485 nm (25 nm bandwidth) and an emission wavelength of 515 nm (10 nm bandwidth). In order to normalize results between experiments, the instrument fluorescence gain setting was adjusted so that the level of EGFP induction by 1 nM TCDD produced a relative fluorescence of 9,000 relative fluorescence units (RFUs). Samples were run in triplicate and the fluorescent activity present in wells containing media only was subtracted from the fluorescent activity in all samples.

### 6.2.4. Cell Proliferatation Analysis

The protocol followed was similar to those previously described in Weiss C, Kolluri S K, Kiefer F and Göttlicher M. (1996). Exp. Cell Res., 226, 154-163, and Kolluri S K, Weiss C, Koff A and Gbttlicher M. (1999). Genes & Dev., 13, 1742-1753. The mouse 5L hepatoma cell line (AhR +/+) and BP8 (an AhR −/− subclone) were kindly provided by Dr. M. Gottlicher (Forschungszentrun Karlsruhe, Institute of Genetics, 76021 Karlsruhe, Germany). Cells were cultured in Dulbecco's modified Eagle medium (DMEM) (Biowhittaker) supplemented with 2 mM L-glutamine (Eurobio), 10% fetal calf serum (FCS), and gentamycin (Gibco BRL) at 37° C. in an atmosphere of 7% CO₂. Indirubin treatments were performed on 50-60% confluent cultures at the indicated time and concentrations (Damiens et al. (2001) Oncogen, 20: 3786-3797). Control experiments were carried out using appropriate dilutions of DMSO.

### 6.2.5. Cell Viability Assay

To quantify the toxicity of indirubins on 5L and BP8 cells, we measured the inhibition of cellular reduction of MTT to MTT formazan according to Mosmann (1983) Immunol. Meth. 65: 55-63. After treatment with indirubins, cells were incubated with 0.5 mg MTT/ml fresh medium at 37° C. for 1 hour. The formazan products were dissolved in DMSO and quantified by measurement of the absorbance at 562 mn.

### 6.2.6. FACS Analysis

Flow cytometry analysis (Coulter EPIX XL2, Beckman, Calif., USA) of cellular DNA content was performed on ethanol-fixed cell suspensions after ribonuclease A III treatment (Sigma) and propidium iodide (Sigma) staining, as previously described (French et al. (1985) Cytometry 6: 47-53). Percentages of cells in G0/G1, S and G2/M phases of cell cycle were then calculated on the basis of DNA distribution histograms provided by the software manufacturer.

### 6.2.7. Indirect Immunofluorescence Microscopy

The wild-type mouse hepatoma cells (Hepa-1) and variant ARNT mutant cells (lacking functional ARNT) were grown in minimum essential medium (InVitrogen, Carlsbad, Calif.) supplemented with 6% FBS (Hyclone Laboratories, Logan, Utah). Cells were routinely maintained in a 37° C. incubator with 5% CO₂. Cells were treated for 90 min. with control vehicle, DMSO or with ligands, TCDD at 10 nM and indirubins at 25 μM. The cells were fixed in 2% paraformaldehyde and processed for indirect immunofluorescence microscopy as previously described (Elbi et al. (2002) Mol. Biol. Cell 13: 2001-2015). Polyclonal anti-AhR primary antibody was used at 1:400 (Biomol Research Laboratories, Plymouth, Pa.). The cells were mounted using Prolong (Molecular Probes, Eugene, Oreg.) and were observed on a Nikon E800 microscope equipped with 63× 1.35 NA, oil immersion Plano Nikon objective and a Photometrics MicroMax cooled CCD camera. Images were collected by using MetaMorph software (Universal Imaging, Downingtown, Pa.).

### 6.3. Example 3

### Identification of Kinase Inhibitory Properties of Indirubins Isolated from a Natural Source and Their 3′-Oxime Derivatives

Each of the compounds isolated from a natural source (compounds 5h, 12a, 5a, and 12b) were synthesized along with its corresponding 3′-oxime derivative (compounds 7h, 13a, 7a, and 13b, respectively), as well as certain 1-methyl derivatives (compounds 13d, 12c, and 13c), 6-bromoindrubin-3′-methoxime (9a) and 6-bromoindrubin-3′-acetoxime (8a), as described above. Following the kinase assay procedures described above (Section 6.2), the effects of these compounds on purified GSK-3α/β, CDK1/cyclin B and CDK5/p25 was determined (Table 1).

As expected, indirubin (5h) was active on GSK-3α/β and on both CDK1 and CDK5 (10-fold less). Although a 6′-bromo substitution (12h) led to reduced kinase inhibition, the 6-bromo substitution (5a) greatly enhanced the selectivity for GSK-3 over both CDK1 and CDK5. Addition of a 3′-oxime substitution (7h, 13a, 7a, and 13b) lead to an overall increase in kinase inhibitory effects and increased solubility, although the selectivity for GSK-3 was slightly reduced. The 6-bromoindirubins substituted on 3′ by methoxime (9a) or acetoxime (8a) were also quite potent and GSK-3 selective (but less soluble, which precluded their use in cells). Methylation on position N1 inactivated the indirubins (13d, 12c, and 13c) as kinase inhibitors.

To further demonstrate that 6-bromoindirubins are selective inhibitors of GSK-3, compounds 6-bromoindirubin-3′-oxime (“BIO”) (7a) and compounds (9a) and (8a) were tested on a series of 20 purified protein kinases, assayed in the presence of 15 μM ATP. The results of these assays confirm the strong selectivity of 6-bromo-substituted indirubins for GSK-3α/β (Table 2). An acetoxime or methoxime substitution on position N3′ further contributes to the GSK-3 selectivity.

### 6.4. Example 4

### Affinity Chromatography on Immobilized Indirubin

Affinity chromatography on immobilized indirubin-3′-oxime was undertaken to demonstrate the selectivity of BIO for GSK-3.

### 6.4.1. Immobilization of Indirubin on a Matrix

Indirubin was synthesized as described above. The indirubin affinity resin compound was then prepared by the oxime formation of indirubin and NH₂—O—(PEG)₃—N₃ with pyridine at 110° C. The azide group was further reduced to amine, and the resulting compound was captured by active ester agarose (Affi-Gel 15, BioRad) (Scheme 3). The completion of the resin capture was monitored by the disappearance of the peak of the indirubin derivative in LC/S (FIGS. 4A and 4B).

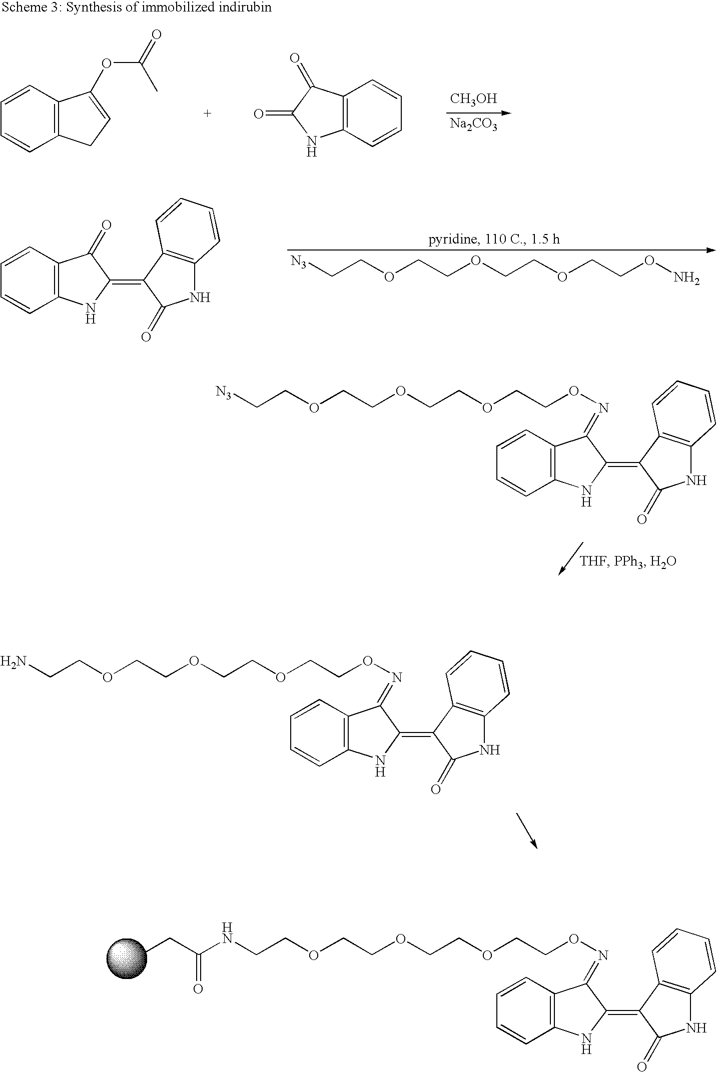

### 6.4.2. Preparation of Extracts and Affinity Chromatography of Interacting Proteins

Porcine brains were obtained from a local slaughterhouse and directly homogenized and processed for affinity chromatography or stored at −80° C. prior to use. Tissues were weighed, homogenized and sonicated in homogenization buffer (2 ml per g of material). Homogenates were centrifuged for 10 min at 14,000 g at 4° C. The supernatant was recovered, assayed for protein content (BIO-Rad assay) and immediately loaded batch wise on the affinity matrix.

Just before use, 20 μl of packed indirubin beads were washed with 1 ml of bead buffer (50 mM Tris pH 7.4, 5 mM NaF, 250 mM NaCt, 5 mM EDTA, 5 mM EGTA, 0.1% Nonidet P-40, 10 μg leupeptin/ml, 10 μg aprotinin/ml, 10 μg soybean trypsin inhibitor/ml and 100 μM benzamidine) and resuspended in 600 μl of this buffer. The tissue extract supernatant (4 mg total protein) was then added in the presence of 20 μM BIO; the tubes were rotated at 4° C. for 30 min. After a brief spin at 10,000 g and removal of the supernatant, the beads were washed four times with bead buffer before addition of 50 μl of 2× Laemmli sample buffer.

### 6.4.3. Electroihoresis and Western Blotting

Following heat denaturation for 3 minutes, the proteins bound to the indirubin matrix were separated by 10% SDS-PAGE (0.7 mm thick gels) followed by immunoblotting analysis or silver staining. Silver staining was performed using a fixative of 250 μl 37% formaldehyde in 250 ml 50% methanol, rinsing with milliQ water containing 35 μM DTT, followed by 0.1% AgNO₃ in milliQ water (w/v), and developing with a solution of 12 g Na₂CO₃ in 400 ml milliQ water containing 200 μl 37% formaldehyde. For immunoblotting, proteins were transferred to 0.1 μm nitrocellulose filters (Schleicher and Schuell). These were blocked with 5% low fat milk in Tris-Buffered Saline -Tween-20 (TBST), incubated with anti-GSK-3α/β (mouse monoclonal anti-GSK-3α/β antibody (KAM-ST002C), from StressGen Biotechnologies Corp.; 1:1000; 1 hr) and analyzed by Enhanced Chemiluminescence (ECL, Amersham).

### 6.4.4. Results of Affinity Chromatography

As described above, porcine brain extract was then run on immobilized indirubin-3′-oxime on Affigel beads in the absence or presence of free BIO, and the bound proteins were resolved by SDS-PAGE, followed by silver staining and anti-GSK-3 Western blotting (FIG. 4C and D). Although a large number of proteins were found to bind to indirubin-beads, the presence of free BIO did not lead to major changes in the overall pattern of indirubin-binding proteins. However it completely prevented the binding of GSK-3α/β, demonstrating the selective effect of BIO for interacting with GSK-3.

### 6.5. Example 5

### Co-Crystal Structure of BIO with GSK-30 and IO With CDK5/p25

A co-crystal structure of BIO with GSK-3β was obtained to determine interactions between the inhibitor and the kinase. For comparison, and as a complement to the CDK2/indirubin co-crystal structures (Hoessel et al. (1999) Nature Cell Biol. 1: 60-67; Davies et al. (2001) Structure 9: 389-397), IO (7h) was co-crystallized with CDK5/p25, another major brain protein kinase involved, along with GSK-3, in the abnormal phosphorylation of tau in Alzheimer's disease.

### 6.5.1. Crystallization of BIO with GSK-3β

Human GSK-3β was cloned, and expressed, in the Bac-to-Bac baculovirus expression system (Life Technologies), as previously described (Dajani et al. (2001) Cell 105: 721-732). Frozen cells from a 5 liter culture were lyzed by thawing, and hand homogenizing on ice in buffer A (50 mM HEPES-NaOH pH 7.5, 300 mM NaCl, 50 mM NaF, 1 mM Na orthovanadate, supplemented with protease inhibitors). The cell extract was centrifuged (48,000 g for 60 min at 4° C.) and the clarified supernatant was mixed with 10 ml Talon metal affinity resin (Clontech) for 2 hr at 4° C. The resin was pelleted by centrifugation at 700 g for 3 min at 4° C., packed into an XK 16/20 column (Amersham Biosciences), washed with 20 column volumes of buffer A, and 20 column volumes of buffer A+5 mM imidazole. The protein was eluted with 50 mM HEPES-NaOH pH 7.0, 300 mM NaCl, 200 mM imidazole, 50 mM NaF, 1 mM Na orthovanadate. 2 mM EDTA and 2 mM DTT were added to the eluted protein, which was then incubated overnight at 4° C. with approximately 3 mg (or 20,000 units) of rTEV protease, to remove the histidine tag. The protein was concentrated to 15 ml using Vivaspin 20 ml centrifugal concentrator (Vivascience), and desalted (HiPrep 26/10 desalting column, Amersham Biosciences) in 50 mM Hepes-NaOH pH 7.0, 300 mM NaCl. The protein was mixed with 10 ml Talon for 2 hr at 4° C. to separate cleaved GSK-3β, from non-cleaved protein, rTEV protease, and other contaminants. 1 mM EDTA and 1 mM DTT were added to the protein, which was concentrated and diluted in ion exchange buffer A (25 mM HEPES-NaOH pH 7.0, 1 mM DTT) to obtain a NaCl concentration of 50 mM. The protein was applied to an 8 ml Source 15S column, HR 10/10 (Amersham Biosciences). The resin was washed with buffer A and the protein was eluted with a 0-500 mM NaCi gradient over 50 column volumes, in 25 mM HEPES-NaOH pH-7.0, 1 mM DTT. The protein was concentrated to ˜10 mg/ml using a 2 ml centricon centrifugal concentrator (Amicon), and the purified GSK-3β was stored at −80° C.

Samples of unphosphorylated and tyrosine phosphorylated GSK-3β (130 μM) were incubated on ice for 1 hr with 200 μM BIO. Crystallization trials were conducted using MDL Structure Screen 1, in 2 μl hanging drop experiments. Unphosphorylated GSK-3β-BIO complex yielded small crystals in several conditions and large crystals with precipitant containing 2.0 M ammonium dihydrogen phosphate, 0.1 M Tris HCl pH 8.5. Crystals large enough for data collection were obtained, by mixing 1 μl of phosphorylated GSK-3pβ-BIO complex, in 25 mM HEPES-NaOH pH 7.0, 250 mM NaCl, 1 mM DTT, with 1 μl precipitant containing 2.0 M ammonium dihydrogen phosphate, 0.1 M Tris HCl pH 8.5. The crystals were stepped through cryo-buffer drops containing 0, 10, 20 and 30% glycerol in under a minute and flash frozen. Data were collected on a single crystal on beamline ID29 at the ESRF, using a wavelength of 0.92A, chosen to highlight the bromine position. Data statistics are summarized in Table 3. Data processing was carried out using MOSFLM and SCALA (Leslie, A. G. W. (1992). Joint CCP4+ESF-EAMCB Newsletter on Protein Crystallography, No. 26.). The structure was solved by molecular replacement methods (Navaza (2001) Acta Crystallogr. D57: 1367-1372) using the GSK-3p structure as search model. Refinement was carried out in CNS (Brunger et al. (1998) Acta Crystallogr. D54: 905-921) using torsion angle molecular dynamics. A model for the bromoindirubin was built and refinement parameters generated from the HIC-UP server (Kleywegt & Jones (1998) Acta Crystallogr. D54: 1119-1131). In the final stages of refinement 125 water molecules were added.

### 6.5.2. Crvstallization of IO With CDK5/p25

A dominant-negative version of the CDK5 kinase containing a point mutation of Asp144 to asparagine was used in the co-crystallization experiments as this mutation substantially improved the expression yields (Tarricone et al. (2001) Molecular Cell 8: 657-669). The CDK5/p25 complex was expressed, purified and crystallized as described previously (Tarricone et al. (2001) Molecular Cell 8: 657-669). After CDK5/p25 crystals had formed, small amounts of indirubin-3′-oxime powder were added to the crystallization drops using a cat's whisker, and soaking was protracted for 2 h at 20° C. During this time, binding of the inhibitor to the crystals could be detected by the coloring of the crystals, which assumed a bright purple color. The crystals were dialyzed overnight in cryo-buffer (mother liquor containing 20% glycerol) as described (Tarricone et al. (2001) Molecular Cell 8: 657-669) and flash-frozen. Data were collected from a single crystal at beamline ID14-1 at ESRF, using a wavelength of 0.93 Å, as summarized in Table 3. Data processing was carried out using programs DENZO and SCALEPACK (Otwinowski (1993) “Oscillation Data Reduction Program” (L. Sawyer, N. Isaacs, and S. Bailey, eds., Warrington, UK: Science and Engineering Research Council/Daresbury Laboratory)). The structure was solved by molecular replacement with the program AMoRe (Navaza (2001) Acta Crystallogr. D57: 1367-1372) using the CDK5/p25 model as a search model. Following rigid body refinement with CNS (Brunger et al. (1998) Acta Crystallogr. D54: 905-921), (Fₒ-F_{c})_{αcalc} map showed clear electron density for the bound inhibitor. After several runs of torsion angle molecular from the Cambridge Structural Database and built into the electron density map. All atoms of indirubin-3′-oxime were restrained to lie on a single plane. The model was subjected to a few final rounds of positional and B factor refinement. Towards the end of refinement, 210 water molecules were added. Data collection, processing and refinement statistics are given in Table 3.

### 6.5.3. Results of Crystallization of BIO with GSK-30 and IO With CDK5/D25

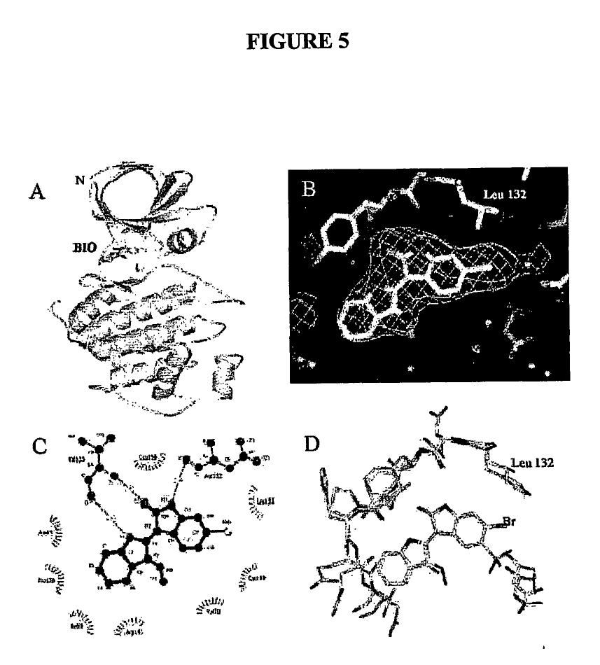

A co-crystal structure was obtained with 2.8 Å resolution (FIG. 5). The overall structure of GSK-3β/BIO is basically identical to the GSK-3β structure recently described (Dajani et al. (2001) Cell 105: 721-732 ; ter Haar et al. (2001) Nat. Stnrct. Biol. 8: 593-596). The small N-terminal lobe of GSK-3β consists predominantly of β-sheets, while the C-terminal lobe is predominantly a-helical (FIG. 5A). BIO is located in the ATP-binding pocket (FIG. 5B). BIO binds in a planar conformation into a narrow hydrophobic pocket whose faces are defined by Ile62, Val70, Ala83, Leu132 and Tyr134 on one side, and Thr138, Arg141, Leu188 and Cys199 on the other. As observed with CDK5-bound indirubin (see below), all direct hydrogen bonding interactions between BIO and the protein are made to the peptide backbone only, and do not involve protein side-chains. The cyclic nitrogen of the pyrole ring donates a hydrogen bond to the peptide carbonyl oxygen of Val135, while the cyclic nitrogen of the lactam ring donates a hydrogen bond to the carbonyl oxygen of Asp133 and the lactam carbonyl oxygen accepts a hydrogen bond from the backbone amide of Val135. As with CDK5, the bond to the backbone amide is quite short at around 2.4 Å. The preference of BIO for binding to GSK-3 as compared to CDKs can be explained by the presence of Leu132 in GSK-3β, which is in van der Waals contact with the bromine at one end of the pocket. In CDK2 and CDK5, the equivalent residue is a phenylalanine whose greater bulk would significantly hinder the binding of a bromine substituent at this position.

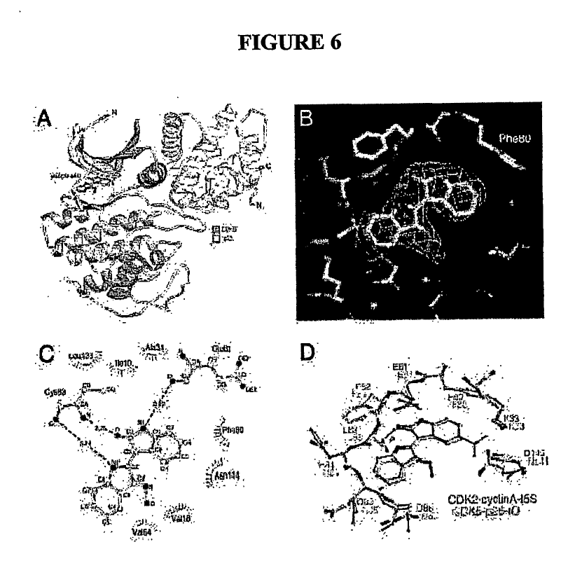

The overall structure of the CDK5/p25-IO complex is basically identical to the CDK5/p25 structure (Tarricone et al. (2001) Molecular Cell 8: 657-669) (FIG. 6A). The small-N-terminal lobe of CDK5 consists predominantly of β-sheets, whilst the C-terminal lobe is predominantly α-helical (FIG. 6A). There are no significant differences in the reciprocal orientation of these lobes between the inhibitor-bound and the native complex. An ‘omit’ map shows clear electron density for all inhibitor atoms (FIG. 6B). IO binds in the ATP-binding pocket of CDK5. The double-ring system of IO is inserted in a hydrophobic pocket formed by CDK5 residues Ile10, Val118, Ala31, Val64, Phe80, Leu133, and the alkyl portion of Asn144, and several van der Waals contacts are observed In addition, three hydrogen bonds are formed with the backbone of CDK5. The N1′ cyclic nitrogen donates a hydrogen bond to the carbonyl oxygen of Cys83^{CDK5}, the carbonyl oxygen of the lactamic ring binds the backbone amide of Cys83^{CDK5}, and the N1 lactam amide nitrogen donates a hydrogen bond to the peptide oxygen of Glu81^{CDK5} (FIG. 6C).

The CDK5 region involved in IO binding was compared with the equivalent CDK2 region of the CDK2/cyclinA/indirubin-5-sulphonate (15S) structure (Davies et al. (2001) Structure 9: 389-397). As shown in FIG. 6D, the aromatic ring systems of both compounds occupy the same position in the two structures. This position provides optimal shape complementarity with the ATP binding cleft while allowing the formation of three hydrogen bonds with the backbone of residues 81 and 83. However, while the oxime group of IO makes no direct interactions with CDK5, the sulphonate group of I5S forms a salt bridge with Lys33^{CDK2} and a hydrogen bond with the backbone nitrogen of Asp₁₄₅^{CDK2}. These additional interactions may explain the higher affinity of I5S for both CDK5/p35 and CDK2/cyclinA (with IC₅₀ values of 0.06 μM and 0.03 μM, respectively) relative to IO (0.10 μM and 0.44 μM respectively) (Hoessel et al. (1999) Nature Cell Biol. 1: 60-67).The nearly identical orientation of these inhibitors indicates that the effect of substitutions at the C3′ and C5 positions may be additive.

### 6.6. Example 6

### Demonstration That 6-Bromoindirubins Act To Inhibit GSK-3 In Cells

The following demonstrates that compounds of the invention behave as GSK-3 modulators in a cellular context. In this exemplification, the effects of BIO on the phosphorylation of specific GSK-3 substrates are presented.

### 6.6.1. Materials and Preparation of Cell Lines

COS1, Hepa (wild-type, CEM/LM AhR deficient and ELB1 ARNT deficient) or SH-SY5Y cells were grown in 6 cm culture dishes in Dulbecco's Modified Medium (DMEM) containing 10% fetal bovine serum (Invitrogen). For treatment, IO (7h) (5 μM, BIO (7a) (5 or 10 μM), MeBIO (13c) (5 or 50 μM), LiCl (20 or 40 mM) or mock solution (DMSO, 0.5% final concentration) was added-to medium when cell density reached ˜70% confluence. After 12 (SH-SY5Y) or 24 hours, the cells, while still in plate, were lysed with lysis buffer (1% SDS, 1 mM sodium orthovanadate, 10 mM Tris pH 7.4). The lysate was passed several times through a 26-gauge needle, centrifuged at 10,000g for 5 min and adjusted to equal protein concentration. About 8 μg of each sample was loaded for immunoblotting. Enhanced chemiluminescence (PerkinElmer) was used for detection. The following primary antibodies were used: mouse anti-beta-catenin CT (Upstate Biotechnolgies, Clone 7D8, recognizes total β-catenin), mouse anti-phospho-p-catenin (Upstate Biotechnologies, Clone 8E7, recognizes dephosphorylated beta-catenin) (van Noort et al. (2002) J. Biol. Chem. 277, 17901-17905), mouse anti-GSK-3 β (BD Transduction Laboratories, 610201), mouse anti-GSK-3 phospho Tyr216 (Transduction Laboratories), rabbit anti-AhR (Aryl hydrocarbon receptor) (BIOMOL Research Laboratories, SA-210), and rabbit anti-actin (Sigma, A5060).

### 6.6.2. Exemplary Results

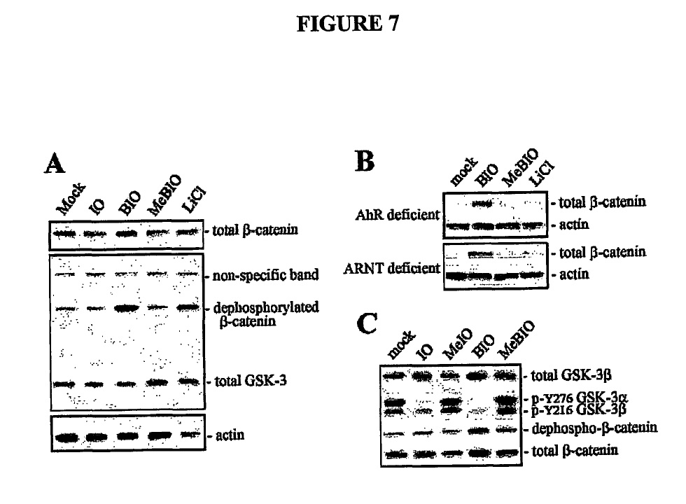

As shown in FIG. 7A, after COS-1 cells were exposed for 24 hrs to various indirubins or to LiCl their levels of unphosphorylated β-catenin, total β-catenin and total GSK-3 were assessed by Western blot using a dephospho-specific monoclonal antibody which cross-reacts with β-catenin when it is not phosphorylated by GSK-3 (on either Ser37 or Thr41), a convenient positive signal on Western blots when GSK-3 is inhibited, and a general anti-β-catenin antibody, as inhibition of β-catenin phosphorylation leads to its stabilization (Huelsken & Birchmeier (2001) Current Opinion in Genetics & Development 11, 547-553). BIO and LiCi treatment, but neither the kinase-inactive MeBIO nor IO, led to a major increase of the unphosphorylated β-catenin level. This was also accompanied by a modest increase in total β-catenin level, as expected from the dephosphorylation-dependent increased half-life of β-catenin. BIO thus acted as a true GSK-3 inhibitor in this cell line.

Indirubins have been recently described to interact with AhR(Adachi et al. (2001) J. Biol. Chem. 276: 31475-31478). To rule out the possibility that BIO-induced β-catenin dephosphorylation/stabilization was due to an indirect, AhR-dependent effect, cell lines deficient either in AhR or ARNT (a co-transcriptional factor of AhR) (Elbi et al. (2002) Mol. Biol. Cell. 13: 2001-2015) were tested. BIO triggered a robust β-catenin stabilization in both cell lines (FIG. 7B). Furthermore, although MeBIO is a potent AhR agonist (see, e.g., TABLE 4 as discussed below), MeBIO is inactive on GSK-3B (Table 1) and on cellular β-catenin phosphorylation/stabilization (FIG. 7). Altogether these data demonstrate that BIO acts by a direct effect on GSK-3 rather than through an indirect, AhR-dependent pathway.

GSK-3α and GSK-3β activity is enhanced by the phosphorylation of a specific tyrosine residue (Tyr276 and Tyr216, respectively) (Hughes et al. (1993) EMBO J. 12: 803-808; Dajani et al. (2003) EMBO J. 22: 494-501). The tyrosine kinase responsible for this phosphorylation in mammals is unknown, but there is some evidence that it indirectly depends on GSK-3 activity (Bhat et al. (2000) Proc. Natl. Acad. Sci. U.S.A. 97: 11074-11079; Shaw et al. (1997) FEBS Lett. 416: 307-311). The effect of compounds of the invention on this key tyrosine phosphorylation was demonstrated. SH-SY5Y cells were exposed to IO, MeIO, BIO and MeBIO for 12 hr and the phosphorylation of Tyr276 (GSK-3α) and Tyr216 (GSK-3β) was estimated by Western blotting with phospho-specific antibodies. As little as 1 μM IO and BIO dramatically reduced the level of tyrosine phosphorylation of both GSK-3 isoforms (FIG. 7C). This was accompanied by an increased level in total GSK-3β, total β-catenin, and dephospho-&catenin. None of these changes were observed with MeIO and MeBIO.

### 6.7. Example 7

### Demonstration That 6-Bromoindirubins Act To Inhibit GSK-3 In Vivo

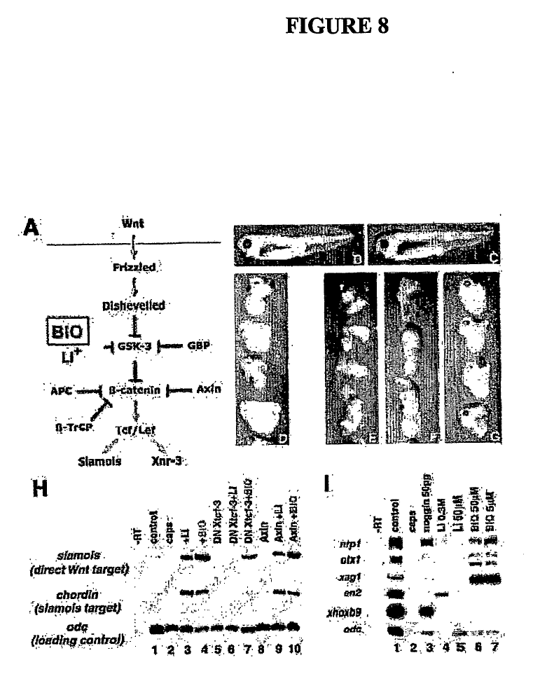

Compounds of the invention modulate GSK-3 in vivo, as exemplified by the effect of BIO in a well-known developmental system. GSK-3 is a component of the Wnt signal transduction pathway, where its phosphorylation of β-catenin inhibits signaling in the absence of Wnt ligand (FIG. 8A) (Huelsken & Birchmeier (2001) Current Opinion in Genetics & Development 11: 547-553). In Xenopus laevis embryos, maternal Wnt activity is necessary for dorsal axis formation, while inhibition of zygotic Wnt activity is required for head formation (Glinka et al. (1997) Nature 389: 517-9).

### 6.7.1. Handling of Embryos

Xenopus laevis embryos obtained by in vitro fertilization were cultured in 0.1×MMR and staged (Nieuwkoop & Faber (1967) Normal table of Xenopus Laevis (North Holland Publishing Co, Amsterdam, The Netherlands)). For lithium treatment embryos were placed in 0.3 M LiCl solution for 10 minutes at the 16 cell stage. For experiments with BIO, the reagent was added to the indicated final concentrations at the 4 cell stage and washed away at stage 8. Embryos were subsequently allowed to develop to tadpole stage. RNA injections were performed at 2-4 cell stage with the indicated amounts of in vitro transcribed RNA in a 10 ml volume. Animal caps were dissected at blastula (stage 9) and cultured to neurula (stage 18) for RT-PCR analysis-.

### 6.7.2. In vitro Transcription and RT-PCR

The RNA expression vectors were pCS2+DN Xtcf-3, encoding amino acids 88-553 of Xtcf-3 (Vonica et al. (2000) Dev. Biol. 217: 230-243), MTPA2 (Zeng et al. (1997) Cell 90: 181-192) for axin. RT-PCR was performed as previously described (Chang et al. (1997) Development 124: 827-837), with the following oligonucleotides: for ODC sense 5′ CAA CGT GTG ATG GGC TGG AT 3′ and antisense 5° CAT AAT AAA GGG TTG GTC TCT GA 3′; for nrp1 sense 5′ GGG TTT CTT GGA ACA AGC 3′ and antisense 5′ ACT GTG CAG GAA CAC AAG 3′; for XAG1 sense 5′ GAG TTG CTT CTC TGG CA 3′ and antisense 5° CTG ACT GTC CGA TCA GAC 3′; for en2 sense 5° CGG AAT TCA TCA GGT CCG AGA TC 3′ and antisense 5′ GCG GAT CCT TTG AAG TGG TCG CG 3′; for Xhoxb9 sense 5′ TAC TTA CGG CGT TGG CTG GA 3′ and antisense 5′ AGC GTG TAA CCA GTT GGC TG 3′; for chordin sense 5° CAG TCA GAT GGA GCA GGA TC 3′ and antisense 5′ AGT CCC ATT GCC CGA GTT GC 3′.

### 6.7.3. Exemplary Results

Phenotypes of embryos treated with BIO or LiCl were compared (FIG. 8B-G). When applied during early cleavage stage, LiCl leads to a hyper dorso-anteriorization at the expense of trunk and tail (anteriorized phenotype, FIG. 8D). BIO phenocopies the LiCl effect in a dose-dependent manner (FIG. 8E-F), while MeBIO remains without effect (FIG. 8C). The Wnt pathway can ectopically induce dorsal genes, like the direct Wnt target siamois (Carnac et al. (1996) Development 122: 3055-3065), and the Spemann organizer marker and siamois target chordin (Kessler (1997) Proc. Natl. Acad. Sci. U.S.A. 94: 13017-22). RT-PCR analysis of animal cap explants treated with LiCl (FIG. 8H, lane 3) or BIO (lane 4) shows induction of both genes. This effect was resistant to injection of RNA encoding the Wnt inhibitor axin (FIG. 8H, lanes 9, 10), which requires GSK-3 for its activity, but not to a dominant negative Tcf molecule (DN Xtcf-3) (lanes 6, 7), which blocks the pathway downstream of GSK-3 (FIG. 8A). Another outcome of ectopic Wnt pathway activation in Xenopus is the indirect induction of neural tissue in animal cap explants (Baker et al. (1999) Genes Dev. 13: 3149-3159). Explants from embryos treated with LiCl (FIG. 8I, lanes 4, 5), BIO (lanes 6, 7), or injected with RNA for the neuralizing factor noggin (lane 3), were compared by RT-PCR for various neural and anterior markers. BIO had the strongest effect on anterior markers like the neural gene otx2 and the cement gland marker xag1 even at the lowest concentration tested (5 μM). As demonstrated, BIO is an effective and specific inhibitor of GSK-3 activity in vivo with higher specific activity than LiCl.

### 6.8. Example 8

### Structural Basis for the Synthesis of Indirubins as Potent and Selective Inhibitors of GSK-3 and CDKs

The identification of 6-bromoindirubins as potent, selective inhibitors of GSK-3 (see, e.g., Section 6.6 above) suggested that this sub-class of indirubins should be further investigated. Aided by the co-crystal structures of various indirubins with CDK2 (Hoessel et al. (1999) Nature Cell Biol. 1: 60-67), CDK2/cyclin A (Davies et al. (2001) Structure 9: 389-397), CDK5/p25 and GSK-3p (Section 6.5 above), the binding of indirubins with the ATP-binding pocket of this kinases was analyzed and modeled. This modeling approach allowed for the understanding of the molecular basis for selectivity and to predict improvements of the indirubin family as kinase inhibitors. Predicted molecules were synthesized and evaluated.

### 6.8.1. Crystallography Results

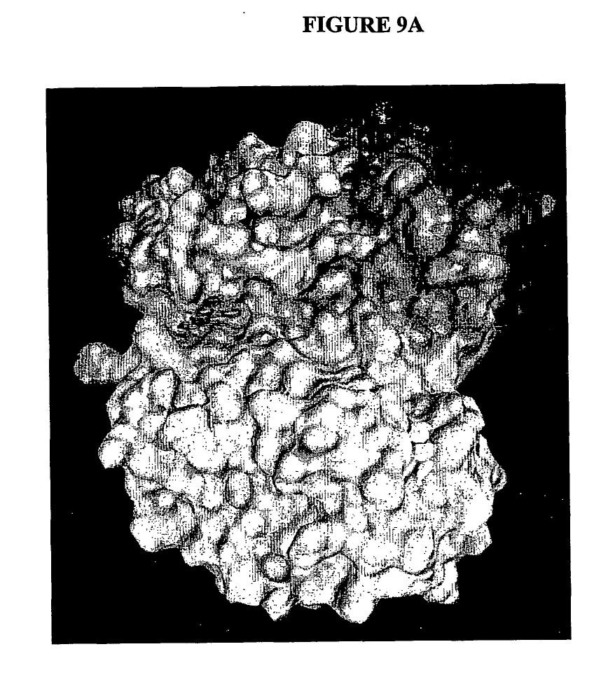

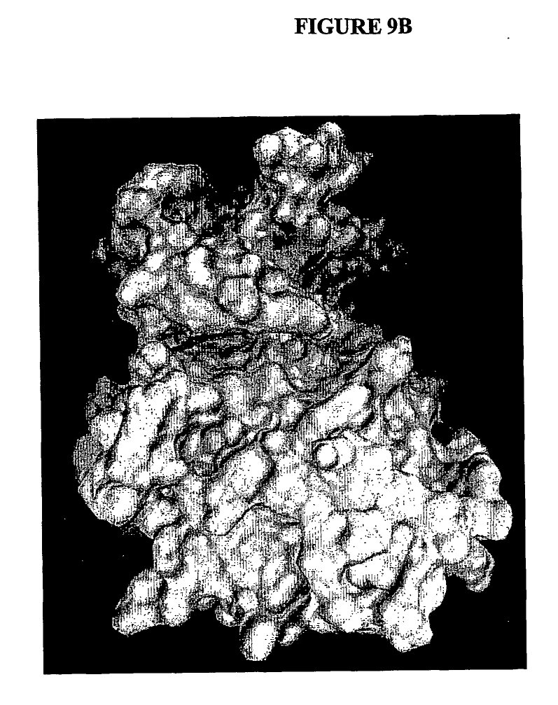

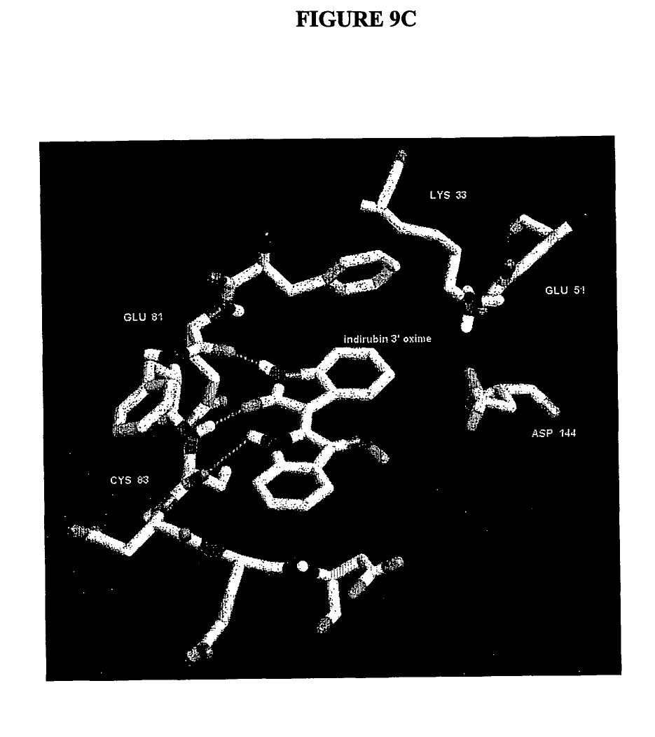

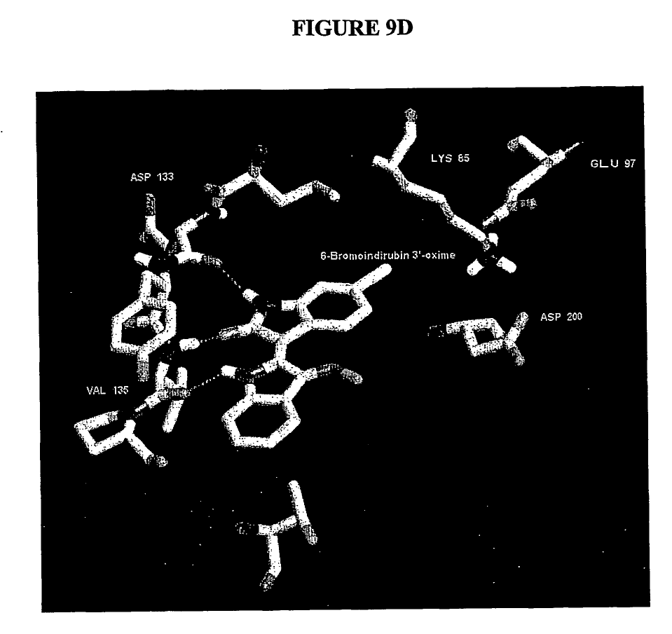

In all cases indirubins are inserted in the ATP-binding pocket located between the small and large lobes of the kinases (FIGS. 9a-d). Comparison of the different structures shows that indirubins adopt a very similar orientation in this enclosed environment.

Substitution at position 6 turned out to be crucial for the selectivity while substitution at 3′ was found to be important for the binding affinity. More specifically, in CDK5 and CDK2, the limits of inner part of the binding cavity are defined by the side chain of Phe80 (FIG. 9c) while in GSK-3 the corresponding residue is Leu132 (FIG. 9d). The difference between the isobutyl side chain of Leu132 and the phenyl ring of Phe80 results in an increase of the pocket width in GSK-3 compared to that of CDK's and can obviously explain the selectivity of 6-bromoindirubin towards GSK-3 compared to CDKs.

An important group of polar residues is located on the same side of the binding cavity: Lys33^{CDK5}/Lys85^{GSK-3}, Asp144^{CDK5}/Asp₂₀₀^{OGSK-3} and Glu51^{CDK5}/Glu97^{GSK-3}, interacting with each other, together with substituents at C5 of indirubin. It has been proposed that these residues play an important role in the ligand-ATP recognition and affinity as Asp interacts with the phosphate hydroxyl group of ATP (De Bondt et al. (1993) Nature 363: 595-602). Moreover the interaction of these th-ee residues could influence the position and flexibility of the Gly loop (residues 12-20) which closes over the end of the binding cleft (Davies (2001) Structure 9: 389-397; Davies et al. (2002) Pharmacol. Ther. 93: 125-133).

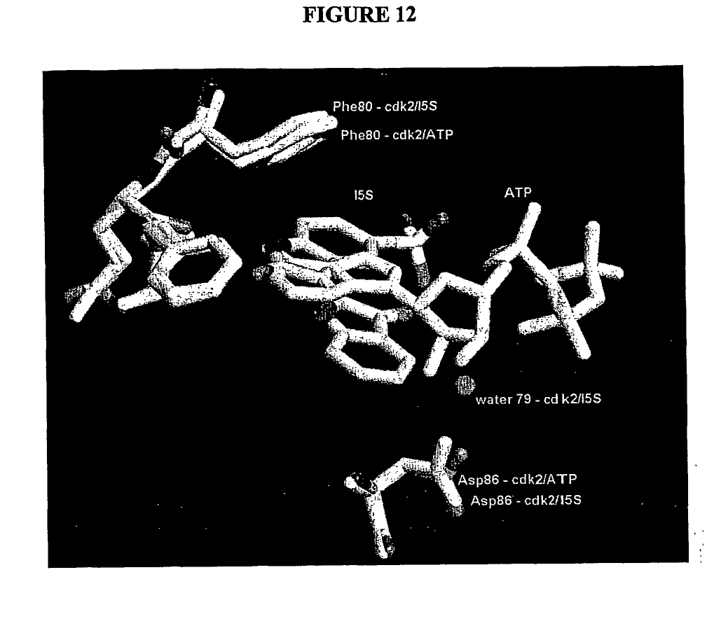

Surprisingly, the C3′ substituent seems to be important in the affinity as it has been noticed that the conversion of the C3′ carbonyl to an oxime improves in inhibitory potency. The crystallographic structure cannot explain this enhancement by any obvious direct interactions of the oxygen or the hydrogen of the oxime with the receptor. A possible explanation could be an indirect interaction of this group with polar residue side chains through water molecules. More specifically it seems that a water molecule is conserved in approximately the same position in both crystal structures of CDK5 (water 79) and CDK2 (water 49) interacting with the oxime group and the C3′ carbonyl oxygen respectively on one side and Asp86 on the other. Moreover this water molecule is located in the same position of ribose hydroxyl oxygen in the CDK2-ATP structure strengthening the above hypothesis (FIG. 12). The same water molecule could also bridge the ligand with the side chain or the backbone carbonyl of Gln130^{CDK2} (Gln131^{CDK5}) through the formation of hydrogen bonds. Asp and Gln are conserved in both ATP-binding sites and they are located at approximately the same distance from the water molecule. In GSK-3β this mode of indirect interactions is not completely preserved as a threonine (Thr138) has replaced Asp86. In that case the crystal structure shows clearly that a direct van der Waals contact exists between the γ-CH3 of Thr138 and the indirubin aromatic system. Moreover a possible link between the oxime and the hydroxyl of threonine would need two bridging water molecules (FIG. 13). Only one of them is located in the same position as the ribose hydroxyl oxygen in ATP complex. However, the backbone carbonyl of Gln185 (homologous to Gln131^{CDK2} and Gln₁₃₀^{CDK5}) located at a distance of 3.3 Å from water 47 and the backbone carbonyl of Ile62 placed at a distance of 3.7 Å from the oxime hydrogen could act as hydrogen bond acceptors of an indirect or direct interaction of the oxime group respectively.

All the above observations were very important for the interpretation of the biological activity obtained from the synthesized molecules and the understanding of their interaction with their molecular target

Among the 6-substituted indirubins the bromo substitution exhibited the highest activity against GSK-3. Calculations showed that this is due mainly to van der Waals interactions which are optimal for both bromo- and iodo- derivatives in agreement with biological test results.

### 6.8.2. Biological Tests

Indirubin analogues were tested against three protein kinases, CDK1/cyclin B, CDK5/p25 and GSK-3α/β following the protocols described above (Section 6.2.2). All assays were run in the presence of 15 μM ATP and appropriate protein substrates (histone H1 for CDK1 and CDK5, GS-1 peptide for GSK-3). IC₅₀ values were determined from dose-response curves and are provided in Table 5 and Table 6.

Among the 6-substituted indirubins the bromo substitution exhibited the highest activity against GSK-3. Calculations showed that this is due mainly to van der Waals interactions which are optimal for both bromo- and iodo- derivatives in agreement with biological test results. In previous studies it had been demonstrated that the derivatives substituted on position 5 (especially with the NO₂ group) exhibited an enhanced inhibitory activity (Leclerc et al. (2001) J. Biol. Chem. 276: 251-260, which is incorporated herein by reference in its entirety). The combination of the substitutions on both positions 5 and 6 resulted in an additive effect on the activity. The IC₅₀ value was reduced about 2 times compared to corresponding single 5- substitution. In agreement with unfavorable steric hindrance between the substituent at C6 and the Phe80 side chain described above, none of the 6- or 5-,6- substituted compounds exhibited any substantial inhibitory activity towards CDK1 or CDK5.

Several 3′-oxime derivatives exhibited an increased inhibitory activity on all three kinases compared to their non-oxime counterparts. Interesting differences were observed between the inhibitory activities against the three kinases of the 3′oxime derivatives. The best inhibitory activity on GSK-3 was observed for the 6-bromo- and, more importantly, the 5-,6-disubstituted compounds, reaching low nanomolar IC₅₀ values. Compared to the corresponding non-oxime derivatives (Table 5), a 5-10 times increase of inhibitory activity was achieved. According to the theoretical model, this is mainly due to favorable contributions of the electrostatic and hydrogen bond energy, suggesting that the interactions of the oxime hydroxyl group play an important role in the binding mode stabilization of these structures.

This activity gain was clearly more pronounced with CDK1 and CDK5 although the overall potency was lower than that seen with GSK-3. This inhibitory response should be attributed to the above mentioned difference between Asp₈₆^{CDK1-5} and the corresponding Thr138^{GSK-3}, and the indirect interaction of these amino acid side chains with the 3′-oxime group through one- or two water molecules. Moreover, another difference was observed in the 3′-oxime derivatives against CDK1 and CDK5. In all cases these derivatives exhibited a higher activity towards CDK5 than to CDK1 although the corresponding non-oxime derivatives were equipotent. There are no obvious structural explanations for this phenomenon but it could be probably attributed to differences in the flexibility of the Gly loop (residues 12-20) as observed in the CDK2 and CDK5 crystal structures. More importantly, the 6-bromo-5-nitro-3′monoxime indirubin derivative displayed an interesting selectivity for CDK5 compared to CDK1.

Position 4 was found to be a disadvantageous site for substitution not only because the size of the substituent is a very restricting factor for the chemical synthesis but also because any substituent (as can be observed by molecular modeling) except hydrogen causes a distortion and the molecule loses its planar structure which is necessary for the binding.

Indirubins bearing nitrogen containing substituents such as amino groups (including various salt forms) and amides at position 5 have also been found to inhibit GSK-3 activity. This include forms either with a substituent at position 6 or with hydrogen, and with or without oxime at position 3′. Table 6 presents IC₅₀ values from kinase assays using exemplary 5-amino-indirubins.

### 6.83. Molecular Modeling

Molecular mechanics docking - scoring calculations were performed to gain a better insight of the interactions between the various synthesized compounds and GSK-3β.

### 6.8.3.1. Molecular Modeling Methods

The 38 ligand molecules (training and test set) were designed and energy-minimized (AMBER*) using MACROMODEL software (Mohamadi et al. (1990) J. Comput. Chem. 11: 440-467). Partial atomic charges were attributed using MOPAC (Stewart (1990) J. Comput.-Aided Mol. Design 4:1-105) (AM1 hamiltonian with EF minimizer and NOMM correction). Solvation energies, entropy corrections and ligand reference energies were calculated for all ligands after individual Monte Carlo minimization using specific built-in PrGen modules. Experimental binding affinities were calculated as relative binding affinities using IC₅₀ values (RBA=(indirubin IC₅₀/ligand IC₅₀)×100). It was approximated that ΔG was about −1 nRBA. Residues in a range of 12A around the ligand were extracted from the receptor Protein Data Bank (PDB) file, all water molecules were removed and all hydrogen atoms were added to the receptor.

The 23 minimized ligand molecules comprising the training set were superimposed over the crystallographic position of 6-Bromo indirubin 3′oxime as appears in the pdb file. The ligand equilibration was performed with the following parameters: Tight_coupling=yes/coupling_constant=1/corr.coupl.RMS=0.197/free_RMS=0.236. For the Monte Carlo search and minimization during equilibration the parameters were: torsion_window: 15 deg./rotation_w.: 5 deg./transl._w.: 0.5 Å/ trials=15/conformers=25/Minimizer: Powell's_conjugate_gradient_{—}1/max.cycles=100/RMS_of_forces=0.1/energy-change=106 /min.step 106. To validate the model the 15 molecules of the test set were introduced into the equilibrated receptor that remained fixed during the following process. Ligands were energy-minimized inside the cavity using Monte Carlo search (torsion_window: 90 deg./rotation_w.: 5 deg./transl._w.: 0.5 Å/trials=15/conformers=40). The prediction of their binding affinities was performed by means of the linear regression obtained by the equilibration procedure of the training set:
ΔG_{predicted}=0.9608·E_{binding}+27.112.
Calculations were carried out on a Silicon Graphics Indigo R4600 workstation.

### 6.8.3.2. Molecular Modeling Results

The relative biological activity was correlated with the calculated interaction energy and a model was constructed allowing the prediction of the affinity of indirubin analogues prior to synthesis. Binding affinities were estimated using PrGen v.2.1 software, (Vedani et al. (1995) J. Am. Chem. Soc. 117: 4987-4994) evaluating ligand - receptor interaction energies, ligand desolvation energies and changes in both ligand-internal energy and ligand internal entropy upon receptor binding according to the formula:
E_{binding}≈E_{ligand-receptor}−TΔS_{binding}−ΔG_{solvation,ligand}+ΔE_{internal,ligand}

Binding energies were coupled with experimental values in order to get a scoring function with high correlation. This was done through an iterative procedure implemented in PrGen referred to as the ligand equilibration protocol. It involved two steps: a) correlation coupled receptor minimization and b) unconstrained ligand relaxation/Monte-Carlo search.

A training set of 23 molecules was subjected to ligand equilibration combined with Monte Carlo search. A total correlation coefficient of 0.902 and an RMS of 0.93 were achieved. The training set was consisted from 11 previously described (Leclerc et al. (2001) J. Biol. Chem. 276: 251-260) molecules and 12 molecules described herein. The model was used to predict the activity of a test set of 15 molecules that were introduced within the equilibrated receptor cavity and subjected to Monte Carlo minimization. The resulting correlation between experimental and predicted binding affinities for both training and test set is presented in FIG. 10 while their ΔG and IC₅₀ values are provided in Table 7. An excellent agreement between calculated and experimental values was observed with an RMS of 1.74. The largest deviation was obtained for 6-Bromo-5-nitroindirubin, which was predicted to be 73 times more potent, while the other 14 ligands of the test set agreed within a factor of 8 or better.

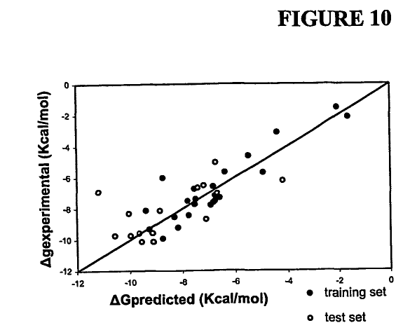

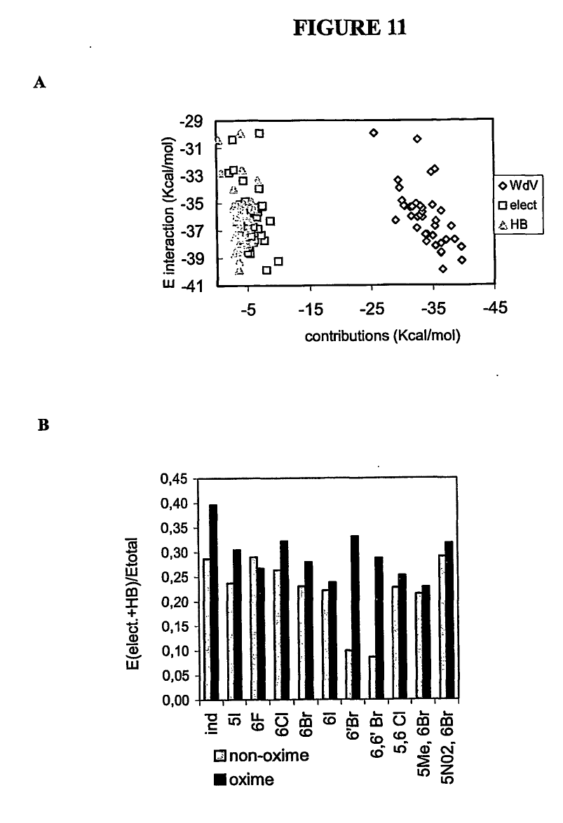

The affinity of indirubins for GSK3 depends mainly on the hydrophobic Van der Waals energy term which accounts for the 66% to 92% of the sum of the three energy terms (VdW, electrostatics, H-bonding) as calculated by PrGen. The significance of VdW contributions on binding is also depicted in FIG. 11a representing each energy term versus the total energy as varied throughout the different ligands. The total interaction energy values vary between −29 to −40 Kcal/mol and the same is true for the VdW term (−25 to −39 Kcal/mol) which appears to influence primarily the total energy. The contributions of the electrostatic (variation from −2 to −10 Kcal/mol) and the H-bond term (variation from −0.3 to −6,7 Kcal/mol) are of less importance and are almost substituent independent. These latter two interactions, however, seem to be in most cases responsible for the improved affinity of the oxime substituted indirubins when compared to their non-oxime analogues. The contribution of the electrostatic and HB term as expressed with the ratio (elect.+HB)/Eww was compared between 11 oximes and the corresponding non-oxime analogues (FIG. 11b). In all cases except 6-F the ratio was greater in oxime analogues. No significant difference was observed concerning the contribution of VdW term, suggesting that the ═N—OH group stabilizing effect is of electrostatic character.

### 6.9. Example 9

### Demonstration That 1-Methyl-Indirubins Selectively Modulate AhR

As revealed by co-crystal structures (see above) the indirubin N1 acts as a H-bond donor in the indirubin/kinase binding. Consequently, methylation at this site should clearly eliminate an essential bonding and inactivate indirubins as kinase inhibitors. Hence, 1-methyl-indirubin (12d), 1-methyl-indirubin-3′-oxime (“MeIO”) (13d), 1-methyl-6-bromo-indirubin (12c) and 1-methyl-6-bromo-indirubin-3′-oxime (“CeBIO”) (13c) were predicted by the molecular modeling described above and found to have selective activity, as they were very potent in the AhR assays while being essentially inactive on CDKs and GSK-3, compared to their non-methylated counterparts, indirubin (IO) and 6-bromo-indirubin-3′-oxime (BIO), respectively.

Following the protocol in Section 6.2.3, human AhR and ARNT were co-expressed stably in yeast and the interaction was coupled to a β-galactosidase reporter system allowing the detection of AhR agonists. Exemplary results demonstrating that 1-methyl-indirubins activate AhR-dependent transcription but do not, or only weakly, inhibit protein kinases is shown in TABLE 4. This property of 1-methyl-indirubins is not shared with indirubins not methylated on N1. As shown in TABLE 4, the effect of indirubin analogues was tested on two protein kinases, CDK1/cyclin B and GSK-3.

For comparison of abilities of compounds to activate AhR-mediated transcription in a yeast cell context to that in a mammalian cell context, the compounds were tested in an assay where ligand and AhR-dependent transcription is measured by the amount of enhanced green fluorescent protein (EGFP) expression which is stably transfected into a mouse hepatoma cell line as described in Section 6.2.3. Using TCDD as a positive control, exemplary results confirmed that indirubins, including indirubin-3′-oxime (“IO”) (7h), the analogue commonly used in cell cycle studies (Damiens et al. (2001) Oncogene 20: 3786-3797; Marko et al. (2001) Br. J. Cancer 84: 283-289), are potent AhR agonists, although much less than TCDD in the mammalian reporter assay (Table 4, FIG. 14).

These results show that indirubins are potent activating AhR ligands in both reporter systems, and that their binding modes to AhR and to protein kinases are unrelated.

### 6.10. Example 10

### Indirubins Trigger Cytoplasm to Nucleus Translocation of AhR

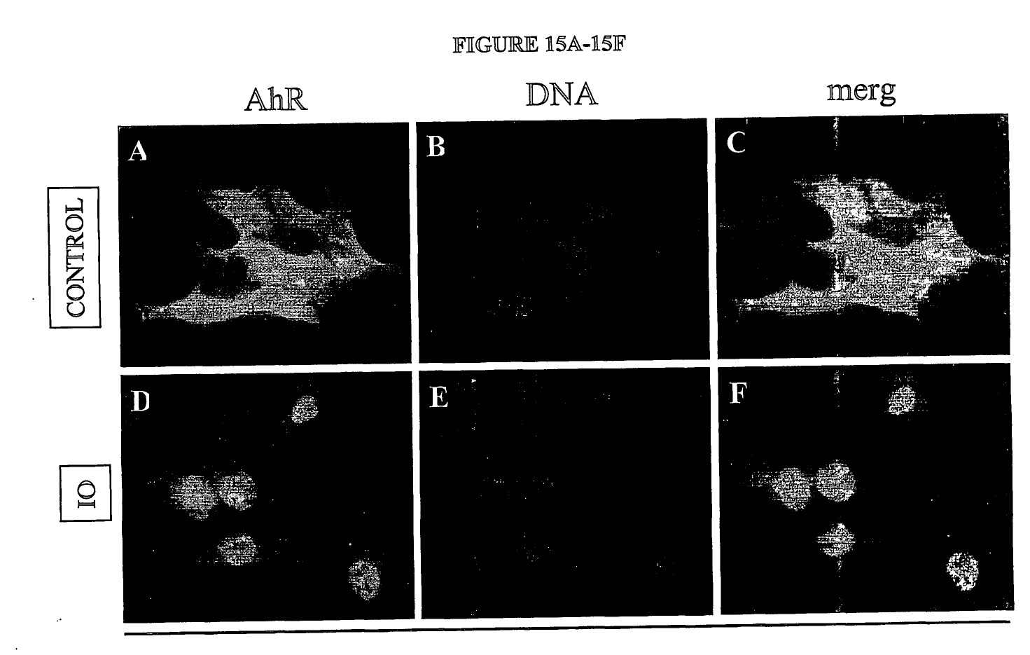

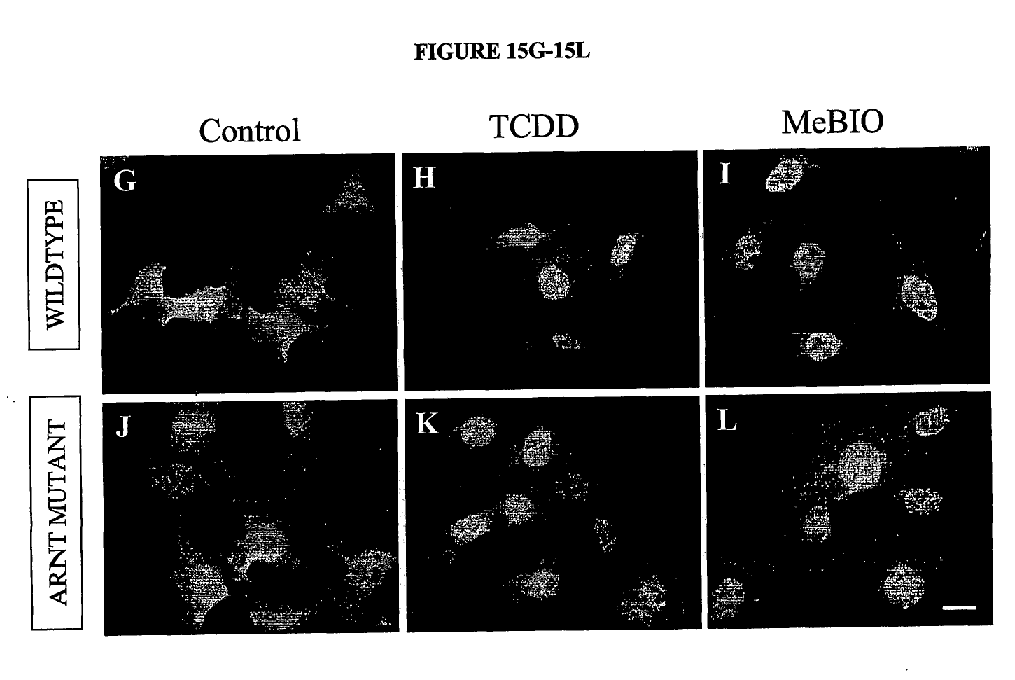

To confirm the interaction of indirubins with AhR in living cells, their effect on AhR intracellular distribution was investigated utilizing immunofluorescence microscopy as described in Section 6.2.7. Nuclear translocation of AhR after treatment with a ligand is a well-known consequence of AhR/ligand interaction (Elbi et al. (2002) Mol. Biol. Cell 13: 2001-2015). Wild-type Hepa-1 and ARNT-mutant cells were treated for 90 min. with various indirubins, or TCDD or DMSO controls. The cells were then fixed and examined by indirect immunofluorescence microscopy following staining with an anti-AhR antibody (FIG. 15). As expected, in the absence of ligand, AhR showed both cytoplasmic and nuclear distribution in wild-type Hepa-1 (FIG. 15 A-C) and in ARNT-mutant cells (FIG. 15 G, J).

Upon addition of IO, AhR rapidly localized to the nucleus (FIG. 15 D-F). MeBIO treatment also caused redistribution of AhR to the nucleus, and the nuclear translocation was independent of the presence of functional ARNT (FIG. 15 I, L). This finding was similar to the nuclear translocation kinetics of AhR observed in TCDD-treated wild-type Hepa-1 cells and ARNT mutant cells (FIG. 15 H, K). All other indirubins tested, such as BIO, MeBIO and 5-iodo-indirubin-3′-oxime, also triggered ARNT-independent nuclear redistribution of AhR. These results demonstrate that indirubins are potent and functional ligands for AhR in living cells and that ARNT is not necessary for indirubin-induced nuclear translocation of AbR.

### 6.11. Example 11

### Survival of AhR −/− and AhR +/+ Cells is Equally Sensitive to Indirubins

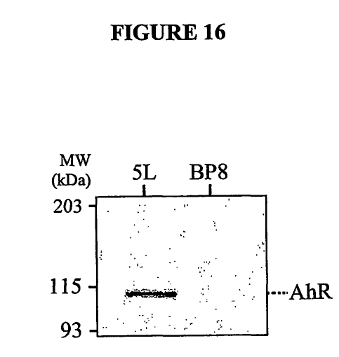

To demonstrate the relative contributions of AhR signaling and kinase inhibition to the cellular effects of indirubins, IO, MeIO, BIO and MeBIO were tested in two mouse hepatoma cell lines, 5L (AhR +/+) and the AhR-deficient sub-clone, BP8 (AhR −/−) following protocols described in Section 6.2.4. Using anti-AhR antibody, AhR expression was detected only in SL cells and not in BP8 cells (FIG. 16).

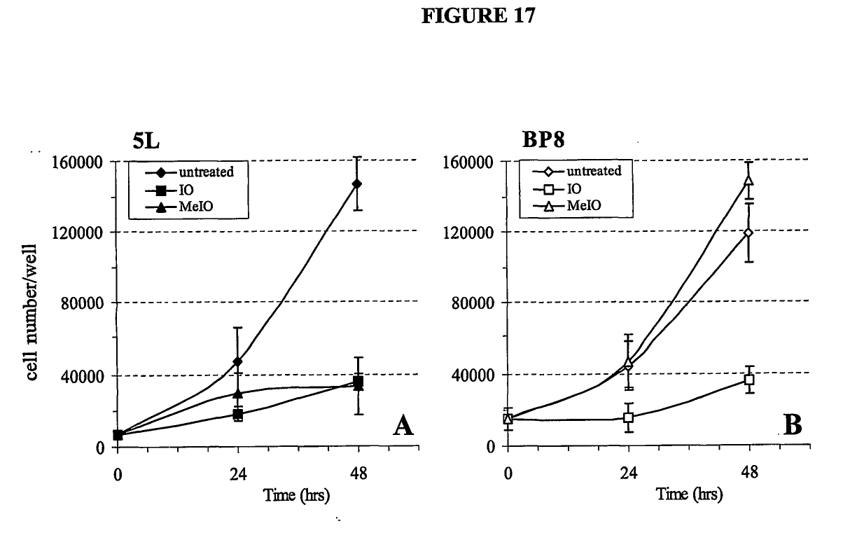

The effects of IO and MeOO (20 μM) on the proliferation rate of 5L and BP8 cell lines was determined by counting directly the number of live cells after 24 and 48 hr of exposure to the indirubins (FIG. 17). This approach clearly showed that IO blocks the proliferation of both cell types, independent of AhR expression. In contrast, MeIO, a potent AhR ligand, but kinase-inactive indimubin, reduced the rate of proliferation only in 5L cells (FIG. 17A), and did not effect the proliferation of AhR −/− cell line BP8 which was, in fact, slightly stimulated (FIG. 17B). This result indicates that MeIO has an AhR-dependent and linase-independent anti-proliferative effect in mouse hepatoma cells.

To investigate the effects of indirubins, 5L and BP8 cells were exposed to a range of concentrations of each indirubin for 24 hr and 48 hr and then the global survival was estimated using the MTT assay, an assay of cell viability based on a mitochondrial enzyme assay, as described in Section 6.2.5. Exemplary results are expressed as a percentage of control, untreated cells. This demonstration illustrates that the survival of both AhR −/− and AhR +/+cells was equally sensitive to IO and BIO, as shown by dose-response curves obtained after 24 and 48 hr exposure to the drugs (FIGS. 18 A, B). Furthermore, the survival of both cell types was apparently not affected by MeIO and MeBIO, the kinase inactive, yet AhR-active, indirubins (FIGS. 18C, D). The possibility remains that MeIO and MeBIO block cell cycle without affecting the mitosis-associated increase in mitochondria number, on which the MTT assay is essentially based. These results indicate that IO and BIO have a kinase-dependent and AhR-independent anti-proliferative effect in mouse hepatoma cells.

### 6.12. Example 12

### AhR-Active Indirubins Trigger a Kinase-Independent t Accumulation of Cells in G1

FACS analysis, as described in Section 6.2.6, was utilized to demonstrate the effects of indirubins on cell cycle distribution of AhR +/+ and −/− cells. Both 5L and BP8 cell lines were treated with 10 μM IO, MeIO, BIO, MeBIO or 100 nM TCDD for 24 hr. The AhR-active indirubins MeIO (FIG. 19AB) and MeBIO (FIG. 19B) induce a very striking accumulation of AhR +/+ cells (5L) but not of AhR −/− cells (BP8) in the G1 phase (FIG. 19B). Comparison of the effects of different indirubins showed that this GI accumulation is modest (but detectable) with IO and BIO, but highly pronounced with the more potent AhR-active indimibins MeIO and MeBIO (FIG. 19A,B). TCDD also triggered an AhR-dependent arrest in G1 (FIG. 19B), as previously reported (Elferink (2003) Progr. Cell Cycle Res. 5: 261-267). Altogether these results show that AhR-active indirubins arrest cells in GI in an AhR-dependent, but kinase-independent manner.

### 6.13. Example 13

### Indirubins Stimulate P²⁷^{KIP1} Expression in an AhR-Dependent Fashion

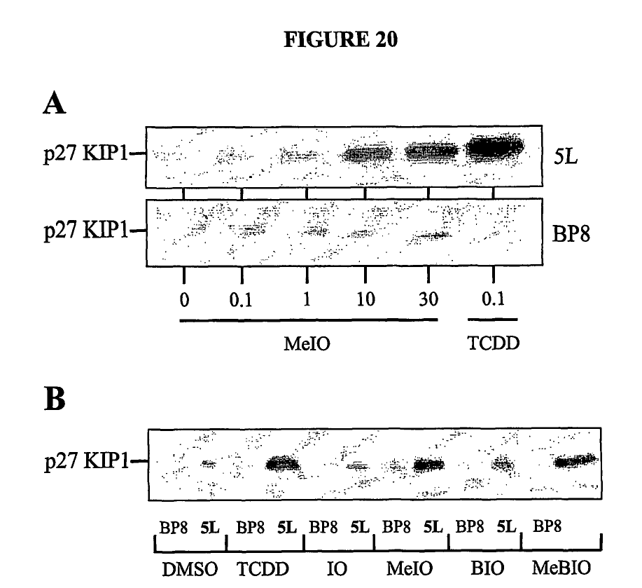

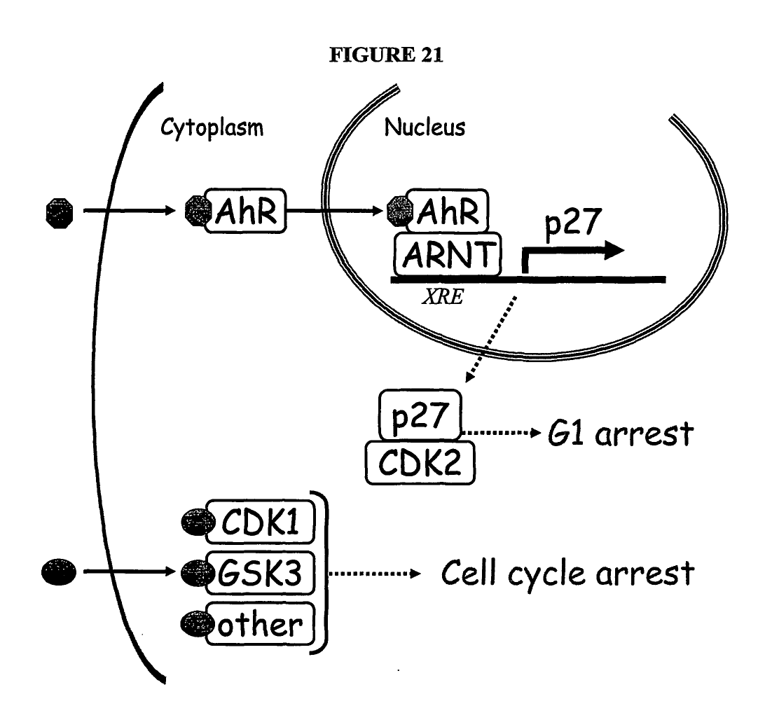

Activation of AhR leads to the enhanced expression of a variety of genes (Elferink (2003) Progr. Cell Cycle Res. 5: 261-267), including the cytochrome P450 CYP1A1 (Santini et al. (2001) J. Pharmacol. Exp. Ther. 299: 718-728) and the CDK inhibitor p27^{KIP1} (Kolluri et al. (1999) Genes & Dev. 13: 1742-1753). Western blotting confirmed that TCDD up-regulates the expression of both CYP1A1 and p₂₇^{KIP1} in the 5L but not in the BP8 cells (FIG. 20). Furthermore, dose-dependent up-regulation of p27^{KIP1} was also found in 5L cells, but not BP8 cells, exposed to the AhR-active MeIO (FIG. 20A). After testing various indirubins, and comparing them to TCDD, it was found that the level of p27^{KIP1} induction correlated significantly with the potency of each indirubin to interact with AhR rather than its ability to inhibit cyclin-dependent kinases (FIG. 20B). Both MeIO and MeBIO were very effective p27^{KIP1} inducers, while IO and BIO induced a modest, but detectable increase in p27^{KIP1} level. These results show that indirubins activate p27^{KIP1} expression in an AhR-dependent, but kinase-independent manner.

TABLE 1
Effects of indirubins on CDK1/cyclin B, CDK5/p25 and GSK-3 kinase activities.
A series of indirubin analogues were tested at various concentrations in three kinase assays.
IC₅₀ values were calculated from the dose-response curves and are reported in μM.

| N^{o} | Compound | GSK-3 α/β | CDK1/cyclin B | CDK5/p25 |
|---|---|---|---|---|
| 5h | indirubin | 1.00 | 10.00 | 10.00 |
| 12h | 6′-bromoindirubin | 22.00 | >100.00 | >100.00 |
| 5a | 6-bromoindirubin | 0.045 | 90.00 | 53.00 |
| 12b | 6,6′-dibromoindirubin | 4.50 | 100.00 | >100.00 |
| 7h | indirubin-3′-oxime (IO) | 0.022 | 0.18 | 0.10 |
| 13a | 6′-bromoindirubin-3′-oxime | 0.34 | 3.00 | 1.20 |
| 7a | 6-bromoindirubin-3′-oxime (BIO) | 0.005 | 0.32 | 0.083 |
| 13b | 6,6′-dibromoindirubin-3′-oxime | 0.12 | 17.00 | 1.30 |
| 13d | 1-methyl-indirubin-3′-oxime (MeIO) | >100.00 | 73.00 | >100.00 |
| 12c | 1-methyl-6-bromoindirubin | >100.00 | 80.00 | >100.00 |
| 13c | 1-methyl-6-bromoindirubin-3′-oxime (MeBIO) | 44.00 | 55.00 | >100.00 |
| 9a | 6-bromoindirubin-3′-methoxime | 0.03 | 3.40 | 2.20 |
| 8a | 6-bromoindirubin-3′-acetoxime | 0.01 | 63.00 | 2.40 |

TABLE 2
Selectivity of 6-bromoindirubins. Kinases were assayed
in the presence of various concentrations of each
compound. IC₅₀ values were determined from the dose-response
curves and are expressed in μM.

| Protein Kinases | 7h (IO)* | 7a (BIO) | 9a | 8a |
|---|---|---|---|---|
| GSK-3α/β | 0.022 | 0.005 | 0.030 | 0.010 |
| CDK1/cyclin B | 0.18 | 0.32 | 3.40 | 63 |
| CDK2/cyclin A | 0.44 | 0.30 | 15 | 4.3 |
| CDK4/cyclin D1 | 3.33 | 10 | >10 | >10 |
| CDK5/p35 | 0.10 | 0.08 | 2.20 | 2.4 |
| erk1 | >100 | >10 | >10 | >10 |
| erk2 | >100 | >10 | >10 | >10 |
| MAPKK | >100 | 10 | >10 | >10 |
| protein kinase C α | 27 | 12 | >10 | >10 |
| protein kinase C β1 | 4 | >10 | >10 | >10 |
| protein kinase C β2 | 20 | >10 | >10 | >10 |
| protein kinase C γ | 8.40 | >10 | >10 | >10 |
| protein kinase C δ | >100 | >10 | >10 | >10 |
| protein kinase C ε | 20 | >10 | >10 | >10 |
| protein kinase C η | 52 | >10 | >10 | >10 |
| protein kinase C ζ | >100 | >10 | >10 | >10 |
| cAMP-dependent PK | 6.3 | >10 | >10 | >10 |
| cGMP-dependent PK | 9 | >10 | >10 | >10 |
| casein kinase 2 | 12 | >10 | 9 | >10 |
| insulin receptor Tyr kinase | 11 | >10 | >10 | >10 |

*data from Hoessel et al. (1999) Nature Cell Biol. 1: 60-67.

TABLE 3
Structure determination of the GSK-3β-BIO and CDK5/p25-IO complexes. Statistics of
the dataset used and of the refined structure.

| | |
|---|---|
| Data Statistics | Refinement Statistics |

GSK-3β-BIO

| | | | |
|---|---|---|---|
| Space group | P4₃2₁2 | Maximal resolution (Å) | 2.8 |
| Resolution (Å) | 2.8 | Protein atoms | 2760 |
| Observations/unique reflections | 220805/50596 | Reflections (working set/test set) | 24570/1204 |
| Completeness (last shell) (%) | 99.7 | R_{cryst}** (%) | 19.25 |
| R_{sym} (last shell) (%) | 7.0 (28.9) | R_{free}*** (%) (% of data) | 22.50 (5) |
| I/σI (last shell) | 18.4 (3.7) | | |
| Unit cell dimensions | a = 98.3 Å | | |
| | c = 198.0 Å | | |

CDK5/p25-IO

| | | | |
|---|---|---|---|
| Space group | C2 | Maximal resolution (Å) | 2.3 |
| Resolution (Å) | 30-2.3 | Protein atoms | 6982 |
| Observations/unique reflections | 220805/50596 | Reflections (working set/test set) | 48066/2530 |
| Completeness (last shell) (%) | 98.8 (99.9) | R_{cryst}** (%) | 24 |
| R_{sym}*(last shell) (%) | 7.5 (39.6) | R_{free}*** (%) (% of data) | 26.5 (5) |
| I/σI (last shell) | 20.4 (4.5) | | |
| Unit cell dimensions | a = 149.5 Å | | |
| | b =90.1 Å | | |
| | c =83.2 Å | | |
| | β = 93.3° | | |

$* {R}_{sym} = \frac{{\sum }_{hkl} {\sum }_{i} {I}_{i} ( hkl ) - \overline{I ( hkl )}}{{\sum }_{hkl} {\sum }_{i} {I}_{i} ( hkl )}$
$** {R}_{cryst} = \frac{{\sum }_{hkl} \vert \vert {F}_{obs} \vert - k \vert {F}_{calc} \vert \vert}{{\sum }_{hkl} \vert {F}_{obs} \vert}$
R_{free} is equivalent to R_{cryst} but is calculated using a disjoint set of reflections excluded from
refinement.

TABLE 4
The effects of indirubins and TCDD on AhR and two protein kinases. A series of
indirubin analogues and TCDD were tested at various concentrations in two AhR interaction
reporter systems and in two kinase assays. EC₅₀ (AhR) and IC₅₀ (kinases) values were
calculated from the dose-response curves and are reported in μM. *, inactive at the indicated
highest concentration tested.

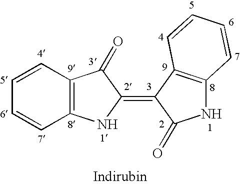

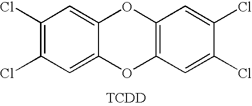

| | | | AhR | | |
|---|---|---|---|---|---|
| | | AhR | (hepatoma | | |
| N° | Compound | (yeast) | cell line) | CDK1/cycin B | GSK-3 |
| 5h | Indirubin | 0.006 | 0.059 | 10.000 | 1.000 |
| 7h | indirubin-3′-oxime (IO) | 0.025 | 0.720 | 0.180 | 0.022 |
| 12d | 1-methyl-indirubin | 0.006 | 0.830 | >100.000 | >100.000 |
| 13d | 1-methyl-indirubin-3′-oxime (MeIO) | 0.008 | 0.129 | 73.000 | >100.000 |
| 6 | 4-chloro-indirubin | >0.100* | 0.310 | 10.000 | >100.000 |
| 15 | 5-methyl-indirubin | 0.040 | 0.025 | 0.280 | 0.062 |
| 16 | 5-bromo-indirubin | 0.035 | 0.007 | 0.230 | 0.055 |
| 17 | 5-chloro-indirubin | 0.040 | 0.052 | 0.280 | 0.050 |
| 18 | 5-iodo-indirubin | 0.030 | 0.030 | 0.220 | 0.068 |
| 19 | 5-iodo-indirubin-3′-oxime | 0.050 | 0.005 | 0.025 | 0.009 |
| 5a | 6-bromo-indirubin | 0.020 | 0.008 | 90.000 | 0.045 |
| 12c | 1-methyl-6-bromo-indirubin | ≧0.100 | 0.050 | >100.000 | >100.000 |
| 7a | 6-bromo-indirubin-3′-oxime (BIO) | 0.009 | 0.720 | 0.320 | 0.005 |
| 13c | 1-methyl-6-bromo-indirubin-3′-oxime (MeBIO) | 0.020 | 0.093 | 92.000 | 44.000 |
| 9a | 6-bromo-indirubin-3′-methoxime | >0.100* | 0.030 | 3.400 | 0.030 |
| 8a | 6-bromo-indirubin-3′-acetoxime | ≧0.100 | 0.160 | 63.000 | 0.010 |
| 5d | 6-chloro-indirubin | 0.030 | 0.007 | >100.000 | 0.140 |
| 7d | 6-chloro-indirubin-3′-oxime | 0.004 | 0.200 | 0.650 | 0.020 |
| 5c | 6-iodo-indirubin | 0.040 | 0.017 | 1.600 | 0.055 |
| 7c | 6-iodo-indirubin-3′-oxime | >0.100* | 0.440 | 1.300 | 0.010 |
| 20 | 5′-bromo-indirubin | ≧0.100 | 0.051 | 0.510 | 0.350 |
| 21 | 5,5′-dibromo-indirubin | >0.200* | 0.047 | 600.000 | 0.250 |
| 12a | 6′-bromo-indirubin | 0.100 | 1.200 | >100.000 | 22.000 |
| 12b | 6,6′-dibromo-indirubin | ≧100 | >10.000 | >100.000 | 4.500 |
| 25 | TCDD | 0.025 | 0.000015 | >15.000 | >15.000 |

TABLE 5
The effects of indirubins on various kinases. A series of indirubin analogues were tested at
various concentrations in three kinase assays. IC₅₀
values (μM) were calculated from the dose-response curves.

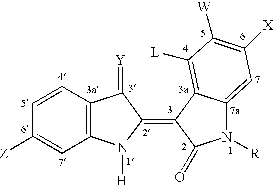

| N° | Compounds | X | Y | Z | W | L | R | CDK1/cyclin B | CDK5/p25 | GSK-3α/β |
|---|---|---|---|---|---|---|---|---|---|---|
| 5h | indirubin | H | O | H | H | H | H | 10.000 | 10.000 | 1.000 |
| 7h | indirubin-3′-oxime | H | NOH | H | H | H | H | 0.180 | 0.100 | 0.022 |
| 8b | indirubin-3′-acetoxime | H | NOAc | H | H | H | H | 1.200 | 0.700 | 0.200 |
| 9b | indirubin-3′-methoxime | H | NOCH₃ | H | H | H | H | 1.000 | 0.400 | 0.150 |
| 12b | 6,6′-dibromoindirubin | Br | O | Br | H | H | H | >100 | >100 | 4.500 |
| 13b | 6,6′-dibromoindirubin-3′-oxime | Br | NOH | Br | H | H | H | 17.000 | 1.300 | 0.120 |
| 12a | 6′-bromoindirubin | H | O | Br | H | H | H | >100 | >100 | 22.00 |
| 13a | 6′-bromoindirubin-3′-oxime | H | NOH | Br | H | H | H | 3.000 | 1.200 | 0.340 |
| 5a | 6-bromoindirubin | Br | O | H | H | H | H | >100 | 53.000 | 0.045 |
| 7a | 6′-bromoindirubin-3′-oxime | Br | NOH | H | H | H | H | 0.320 | 0.083 | 0.005 |
| 8a | 6-bromoindirubin-3′-acetoxime | Br | NOAc | H | H | H | H | 63.000 | 2.400 | 0.010 |
| 9a | 6-bromoindirubin-3′-methoxime | Br | NOCH₃ | H | H | H | H | 3.700 | 2.200 | 0.030 |
| 12c | 6-bromo-N-methylindirubin | Br | O | H | H | H | CH₃ | >100 | >100 | >100 |
| 13c | 6-bromo-N-methyl-indirubin-3′-oxime, | Br | NOH | H | H | H | CH₃ | 92.000 | >100 | >100 |
| 5d | 6-chloroindirubin | Cl | O | H | H | H | H | >100 | >100 | 0.140 |
| 7d | 6-chloroindirubin-3′-oxime | Cl | NOH | H | H | H | H | 0.650 | 0.100 | 0.020 |
| 8d | 6-chloroindirubin-3′-acetoxime | Cl | NOAc | H | H | H | H | 30.000 | 0.200 | 0.017 |
| 5c | 6-iodoindirubin | I | O | H | H | H | H | 1.600 | 5.300 | 0.055 |
| 7c | 6-iodoindirubin-3′-oxime | I | NOH | H | H | H | H | 1.300 | 0.300 | 0.010 |
| 8c | 6-iodoindirubin-3′-acetoxime | I | NOAc | H | H | H | H | 2.200 | 1.300 | 0.013 |
| 5i | 6-vinylindirubin | CH═CH₂ | O | H | H | H | H | 4.200 | 2.400 | 0.240 |
| 7i | 6-vinylindirubin-3′-oxime | CH═CH₂ | NOH | H | H | H | H | 1.200 | 0.420 | 0.060 |
| 8i | 6-vinylindirubin-3′-acetoxime | CH═CH₂ | NOAc | H | H | H | H | 1.600 | 0.400 | 0.065 |
| 5b | 6-fluoroindirubin | F | O | H | H | H | H | 1.500 | 1.000 | 0.650 |
| 7b | 6-fluorooindinibin-3′-oxime | F | NOH | H | H | H | H | 0.320 | 0.150 | 0.130 |
| 8b | 6-fluoroindirubin-3′-acetoxime | F | NOAc | H | H | H | H | 0.600 | 0.300 | 0.090 |
| 12d | N-methylindirubin | H | O | H | H | H | CH₃ | >100 | >100 | >100 |
| 13d | N-methylindirubin-3′-oxime | H | NOH | H | H | H | CH₃ | 73.000 | >100 | >100 |
| 5f | 6-bromo-5-methylindirubin | Br | O | H | CH₃ | H | H | 30.000 | 60.000 | 0.025 |
| 7f | 6-bromo-5-methylindirubin-3′-oxime | Br | NOH | H | CH₃ | H | H | 0.300 | 0.130 | 0.006 |
| 8f | 6-Bromo-5-methylindirubin-3′-acetoxime | Br | NOAc | H | CH₃ | H | H | 31.000 | 30.000 | 0.007 |
| 5e | 6,5-dichloroindirubin | Cl | O | H | Cl | H | H | 45.000 | 60.000 | 0.030 |
| 7e | 6,5-dichloroindirubin-3′-oxime | Cl | NOH | H | Cl | H | H | 0.140 | 0.060 | 0.004 |
| 8e | 6,5-dichloroindirubin-3′-acetoxime | Cl | NOAc | H | Cl | H | H | 30.000 | 0.100 | 0.004 |
| 5g | 6-Bromo-5-nitroindirubin | Br | O | H | NO₂ | H | H | >100 | >100 | 0.100 |
| 7g | 6-Bromo-5-nitroindirubin-3′-oxime | Br | NOH | H | NO₂ | H | H | 12.000 | 0.150 | 0.007 |
| 8g | 6-Bromo-5-nitroindirubin-3′-acetoxime | Br | NOH | H | NO₂ | H | H | 11.000 | 31.000 | 0.006 |
| 6 | 4-chloroindirubin | H | O | H | H | Cl | H | 10.000 | 10.000 | >100 |

TABLE 6
The effects of 5-amino-indirubins on CDK-5 and GSK-3

| | CDK-5 | GSK-3 |
|---|---|---|
| Compound | (IC50 μM) | (IC50 μM) |
| 5-amino-indirubin (23) | 2.6 | 1.5 |
| 5-amino-3′-oxime indirubin (24) | 0.47 | 0.75 |
| 6-bromo-5-amino-indirubin (27) | 0.6 | 0.054 |
| 6-bromo-5-amino-3′-oxime | 0.57 | 0.056 |
| indirubin (28) | | |

TABLE 7
Experimental and predicted lnRBA and IC₅₀ values
of the training and test sets of indirubins.

| | | ΔGexp | ΔGpred | IC₅₀exp | IC₅₀pred |
|---|---|---|---|---|---|
| | Ligand | (Kcal/mol) | (Kcal/mol) | (μM) | (μM) |
| TRAIN- | Indirubin | −4.605 | −5.490 | 1.000 | 0.413 |
| ING | Indirubin-3′-oxime | −8.422 | −7.761 | 0.022 | 0.043 |
| SET | 5,5′-Dibromoindirubin | −5.991 | −8.748 | 0.250 | 0.016 |
| | 5-Bromoindirubin | −7.505 | −6.768 | 0.055 | 0.115 |
| | 5-Chloroindirubin | −7.601 | −6.829 | 0.050 | 0.108 |
| | 5-Fluoroindirubin | −7.156 | −6.768 | 0.078 | 0.115 |
| | 5-Iodoindirubin-3′-oxime | −9.316 | −9.250 | 0.009 | 0.010 |
| | 5-Iodoindirubin | −7.293 | −6.590 | 0.068 | 0.137 |
| | 5-Methylindirubin | −7.386 | −7.493 | 0.062 | 0.056 |
| | 5-Nitroindirubin | −7.775 | −6.930 | 0.042 | 0.098 |
| | 5′-Bromoindirubin | −5.655 | −6.395 | 0.350 | 0.167 |
| | 6,6′-Dibromoindirubin | −3.101 | −4.395 | 4.500 | 1.234 |
| | 6,6′-Dibromoindirubin-3′-oxime | −6.725 | −7.550 | 0.120 | 0.053 |
| | 6-Bromoindirubin 6Br | −7.706 | −7.533 | 0.045 | 0.054 |
| | 6-Bromoindirubini-3′-acetoxime | −9.210 | −8.181 | 0.010 | 0.028 |
| | 6-Bromoindirubin-3′-methoxime | −8.112 | −9.369 | 0.030 | 0.009 |
| | 6-Bromoindirubin-3′-oxime | −9.903 | −8.753 | 0.005 | 0.016 |
| | 6-Chloroindirubin | −6.571 | −6.844 | 0.140 | 0.107 |
| | 6-Chloroindirubin-3′-oxime | −8.517 | −8.309 | 0.020 | 0.025 |
| | 6-Iodoindirubin | −7.506 | −7.801 | 0.055 | 0.041 |
| | 6-Iodoindirubin-3′-oxime | −9.210 | −8.173 | 0.010 | 0.028 |
| | 6'-Bromoindirubin | −1.514 | −2.068 | 22.000 | 12.644 |
| | 6'-Bromoindirubin-3′-oxime | −5.684 | −4.955 | 0.340 | 0.705 |
| TEST | 5,6-Dichloroindirubin | −8.110 | −8.850 | 0.030 | 0.014 |
| SET | 5,6-Dichloroindirubin-3′-oxime | −10.120 | −9.542 | 0.004 | 0.007 |
| | 6-Bromo-5-methylindirubin | −8.290 | −10.037 | 0.025 | 0.004 |
| | 6-Bromo-5-methylindirubin-3′-oxime | −9.720 | −9.981 | 0.006 | 0.004 |
| | 6-Bromo-5-nitroindirubin-3′-oxime | −9.560 | −9.131 | 0.007 | 0.010 |
| | 6-Bromo-5-nitroindirubin | −6.900 | −11.186 | 0.100 | 0.001 |
| | 6-Fluoroindirubin | −5.030 | −6.749 | 0.650 | 0.117 |
| | 6-Fluoroindirubin-3′-oxime | −6.640 | −7.421 | 0.130 | 0.059 |
| | 6-Fluoroindirubin-3′-acetoxime | −7.010 | −6.698 | 0.090 | 0.123 |
| | Indirubin-3′-acetoxime | −6.220 | −4.199 | 0.200 | 1.501 |
| | Indirubin-3′-methoxime | −6.500 | −7.195 | 0.150 | 0.075 |
| | 6-Chloroindirubin-3′-acetoxime | −8.680 | −7.107 | 0.017 | 0.082 |
| | 5,6-Dichloroindirubin-3′-acetoxime | −10.130 | −9.102 | 0.004 | 0.011 |
| | 6-Bromo-5-methylindirubin-3′-acetoxime | −9.570 | −9.632 | 0.007 | 0.006 |
| | 6-Bromo-5-nitroindirubin-3′-acetoxime | −9.720 | −10.580 | 0.006 | 0.003 |

We claim:

1. An isolated compound comprising an indirubin molecule substituted with a halogen at position C6 of the indirubin molecule.

2. The isolated compound of claim 1, wherein the compound inhibits GSK-3 activity with an IC₅₀ value of less than 0.1 μM using GS-1 peptide as a substrate.

3. A pharmaceutically acceptable salt or solvate of the isolated compound of claim 1.

4. The isolated compound of claim 1, wherein the compound is selected from the group consisting of 6-bromoindirubin (5a), 6,6′-dibromoindimibin (12b), 6-bromoindirubin-3′-oxime (“BIO”) (7a), 6,6′-dibromnoindirubin-3′-oxime (13b), 6-bromoindirubin-3′-methoxime (9a), 6-bromo-5-methylindirubin (51) and 6-bromoindirubin-3′-acetoxime (8a), and pharmaceutically acceptable salts thereof

5. The isolated compound of claim 1, wherein the compound is selected from the group consisting of 6-bromo-5-aminoindirubin (27) and 6-bromo-5-amino-3′-oxime-indirubin (28) and pharmaceutically acceptable salts thereof.

6. A compound selected from the group of 6-bromoindirubin (5a), 6,6′-dibromoindirubin (12b), 6-bromoindirubin-3′-oxime (“BIO”) (7a), 6,6′-dibromoindirubin-3′-oxime (13b), 6-bromoindirubin-3′-methoxime (9a), 6-bromo-5-methylindirubin (51), 6-bromo-5-aminoindirubin (27), 6-bromo-5-amino-3′-oxime-indirubin (28), 6-bromoindirubin-3′-acetoxime (8a), 5-amino-indirubin (23), 5-amino-3′-oxime-indirubin (24) and pharmaceutically acceptable salts thereof.

7. An inhibitor of GSK-3 activity, wherein the inhibitor comprises an indirubin molecule with a halogen atom covalently attached to carbon number 6, and the inhibitor inhibits GSK-3 activity with an IC₅₀ below 5 μM in an activity assay using GS-1 peptide as a substrate.

8. The inhibitor of claim 7, wherein the halogen atom is bromine.

9. A pharmaceutical composition comprising 6-bromoindirubin, or pharmaceutically acceptable salt thereof, and a pharmaceutically acceptable vehicle.

10. A method of inhibiting GSK-3 activity comprising contacting GSK-3 with the inhibitor of claim 7.

11. The method of claim 10, wherein the GSK-3 activity is inhibited in vitro.

12. The method of claim 10, wherein the GSK-3 activity is inhibited in a cell.

13. The method of claim 10, wherein the inhibitor is selected from group of compounds consisting of 6-bromoindirubin (5a), 6,6′-dibromoindirubin (12b), 6-bromoindirubin-3′-oxime (7a), 6,6′-dibromoindirubin-3′-oxime (13b), 6-bromoindirubin-3′-methoxime (9a), 6-bromoindirubin-3′-acetoxime (8a), 6-iodoindirubin (5c), 6-iodoindirubin-3′-oxime (7c) and 6-iodoindirubin-3′-acetoxime (8c), and pharmaceutically acceptable salts thereof

14. The method of claim 10, wherein the inhibitor is selected from group of compounds consisting of 6-bromo-5-aminoindirubin (27), 6-bromo-5-amino-3′-oxime-indirubin (28), and pharmaceutically acceptable salts thereof.

15. A method for the isolation of 6-bromoindirubin from a natural source comprising the steps described in Example 6.1.2.

16. A method for preventing, treating or ameliorating type 2 diabetes or Alzheimer's disease in a mammal, comprising administering to the mammal an effective disease-treating or condition-treating amount of a pharmaceutical composition of indirubin-type compound consisting of 6-bromoindirubin (5a) or a 6-bromoindirubin derivative or analogue.
